SEQUENCE LISTING (33 sequences)

SEQ ID NO: 1 - protein, 31 aa, Homo sapiens
EAEDLQVGQVELGGGPGAGSLQPLALEGSLQ

SEQ ID NO: 2 - protein, 31 aa, Pan troglodytes; identical to SEQ ID NO: 1

SEQ ID NO: 3 - protein, 31 aa, Gorilla gorilla; identical to SEQ ID NO: 1

SEQ ID NO: 4 - protein, 31 aa, Pongo pygmaeus; identical to SEQ ID NO: 1

SEQ ID NO: 5 - protein, 31 aa, Chlorocebus aethiops
EAEDPQVGQVELGGGPGAGSLQPLALEGSLQ

SEQ ID NO: 6 - protein, 31 aa, Canis lupus familiaris
EVEDLQVRDVELAGAPGEGGLQPLALEGALQ

SEQ ID NO: 7 - protein, 31 aa, Oryctolagus cuniculus
EVEELQVGQAELGGGPDAGGLQPSALELALQ

SEQ ID NO: 8 - protein, 31 aa, Rattus norvegicus
EVEDPQVAQLELGGGPGAGDLQTLALEVARQ

SEQ ID NO: 9 - protein, 31 aa, Apodemus semotus; identical to SEQ ID NO: 8

SEQ ID NO: 10 - protein, 31 aa, Geodia cydonium
EVEDPQVGQVELGAGPGAGSEQTLALEVARQ

SEQ ID NO: 11 - protein, 27 aa, Mus musculus
EVEDPQVAQLELGGGPGAGDLQTLALE

SEQ ID NO: 12 - protein, 27 aa, Mus caroli
EVEDPQVAQLELGGGPGAGDLQTLALE

SEQ ID NO: 13 - protein, 31 aa, Rattus norvegicus
EVEDPQVPQLELGGGPGAGDLQTLALEVARQ

SEQ ID NO: 14 - protein, 31 aa, Rattus losea
EVEDPQVAQQELGGGPGAGDLQTLALEVARQ

SEQ ID NO: 15 - protein, 31 aa, Niviventer coxingi
EVEDPQVPQLELGGGPGTGDLQTLALEVARQ

SEQ ID NO: 16 - protein, 26 aa, Microtus kikuchii
VEDPQVAQLELGGGPGAGDLQTLALE

SEQ ID NO: 17 - protein, 31 aa, Rattus norvegicus
EVEDPQVPQLELGGGPEAGDLQTLALEVARQ

SEQ ID NO: 18 - protein, 31 aa, Felis catus
EAEDLQGKDAELGEAPGAGGLQPSALEAPLQ

SEQ ID NO: 19 - protein, 26 aa, Mesocricetus auratus
VEDPQVAQLELGGGPGADDLQTLALE

SEQ ID NO: 20 - protein, 31 aa, Niviventer coxingi
EVEDPQVAQLELGEGPEAGDLQTLALEVARQ

SEQ ID NO: 21 - protein, 31 aa, Apodemus semotus
EVEDPQVEQLELGGAPGTGDLETLALEVARQ

SEQ ID NO: 22 - protein, 31 aa, Rattus losea
EVEDPQVPQLELGGSPEAGDLQTLALEVARQ

SEQ ID NO: 23 - protein, 30 aa, Meriones unguiculatus
VEDPQMPQLELGGSPGAGDLQALALEVARQ

SEQ ID NO: 24 - protein, 30 aa, Psammomys obesus
VDDPQMPQLELGGSPGAGDLRALALEVARQ

SEQ ID NO: 25 - protein, 26 aa, Sus scrofa
EAENPQAGAVELGGGLGGLQALALEG

SEQ ID NO: 26 - protein, 31 aa, Rhinolophus ferrumequinum
EVEDPQAGQVELGGGPGTGGLQSLALEGPPQ

SEQ ID NO: 27 - protein, 31 aa, Equus przewalskii
EAEDPQVGEVELGGGPGLGGLQPLALAGPQQ

SEQ ID NO: 28 - protein, 26 aa, Bos taurus
EVEGPQVGALELAGGPGAGGLEGPPQ

SEQ ID NO: 29 - protein, 31 aa, Otolemur garnettii
DTEDPQVGQVGLGGSPITGDLQSLALDVPPQ

SEQ ID NO: 30 - protein, 31 aa, Artificial Sequence
  Synthesized
EXEXXQXXXXELXXXXXXXXXXXXALBXXXQ

SEQ ID NO: 31 - protein, 5 aa, Artificial Sequence
  Synthesized
EGSLQ

SEQ ID NO: 32 - protein, 12 aa, Artificial Sequence
  Synthesized
EAEDLQVGQVEL

SEQ ID NO: 33 - protein, 31 aa, Artificial Sequence
  Synthesized
GXEXXQXXXXELXXXXXXXXXXXXALBXXXQ

(19) United States
(12) Patent Application Publication
(10) Pub. No.: US 2011/0190192 A1
(43) Pub. Date: Aug. 4, 2011

(54) METHODS FOR TREATING ERECTILE DYSFUNCTION IN PATIENTS WITH INSULIN-DEPENDENT DIABETES

(75) Inventor: John Wahren, Djursholm (SE)

(73) Assignee: Cebix Inc., LaJolla, CA (US)

(21) Appl. No.: 12/967,491

(22) Filed: Dec. 14, 2010

Related U.S. Application Data
(60) Provisional application No. 61/286,666, filed on Dec. 15, 2009.

(51) Int. Cl. A61K 38/17; A61P 15/10

(52) U.S. Cl. 514/1.1

(57) ABSTRACT

The present invention relates to the development of improved methods for treating erectile dysfunction associated with diabetes. Significantly, such dosing regimens can be combined with established methods for treating sexual dysfunction, including PDE5 inhibitors such as those sold under the trademark VIAGRA® to provide for significantly improved efficacy compared to the PDE5 inhibitor alone.

### CROSS-REFERENCE TO RELATED APPLICATIONS

This application claims the benefit of U.S. provisional patent application No. 61/286,666 filed on Dec. 15, 2009, the entire contents of which are incorporated herein by reference.

### STATEMENT REGARDING FEDERALLY SPONSORED RESEARCH OR DEVELOPMENT

Not Applicable.

### TECHNICAL FIELD

The present invention relates to the development of improved methods for treating patients with diabetes who have erectile dysfunction based on the administration of a therapeutic dose of C-peptide.

### BACKGROUND OF THE INVENTION

Proper sexual functioning depends on progression through the normal sexual response cycle which may be divided into four phases. 1); The desire phase, which consists typically of fantasies about and the desire to have sexual activity. 2); The excitement phase, which is characterized by the subjective sense of sexual pleasure and accompanying physiological changes, namely penile tumescence and erection in men; and pelvic congestion, swelling of the external genitalia, and vaginal lubrication and expansion in woman. 3); The orgasmic phase, where sexual pleasure peaks with the release of sexual tension and rhythmic contraction of the perineal muscles and reproductive organs. In men, the sensation of ejaculatory inevitability is followed by the ejaculation of semen. In woman, contractions of the outer third of the vaginal wall occur. 4); The final phase, resolution, which is characterized by a sense of muscular relaxation and general well-being. Men are physiologically refractory to erection and orgasm for a variable period, whereas women may be able to respond to further stimulation. Disorders of the sexual response can occur at one or more of these phases, and are common among both male and female populations. In men disorders of sexual function (i.e., sexual disorders or sexual dysfunction) include erectile dysfunctions, ejaculatory dysfunctions and hypoactive sexual desire disorders.

Variations in intensity make erectile dysfunction and its incidence in the male population difficult to define. Recent estimates suggest that the number of U.S. men with erectile dysfunction (ED) may be near 10 to 20 million, and inclusion of individuals with partial ED increases the estimate to about 30 million. ED has a number of etiologies, including neuropathy and vascular disease. There is also a high incidence of erectile insufficiency among diabetics, particularly those with insulin-dependent diabetes (Penson D F. et al., J. Sex. Med. 6(7) 1969-1978 (2009). About half of diabetic males suffer from erectile insufficiency, and about half of the cases of neurogenic impotence are in diabetics. (Chitaley. K., J. Sex. Med. S3 262-268 (2009)).

Various regimes are available for treatment of sexual dysfunction in men, and include agents that act vasodilatory on erectile tissue (e.g., adrenoceptor blocking agents, apomorphine, prostaglandins, organic nitrates, L-arginine, minoxidil, potassium channel openers, rho-kinase inhibitors, testosterone gels, and derivatives such as testosterone undecanoate, phosphodiesterase inhibitors) and drugs that act centrally in the brain or spinal cord such as yohimbine, opioid receptor antagonists, dopamine receptor agonists, antidepressants, therapies that elevate serotonin and dopamine levels, and melanocortin receptor agonists. Additionally devices and procedures such as vascular extracorporeal shockwave therapy (Vascuspec) and the use of infrared radiation have been employed. However, such approaches present significant drawbacks.

Currently, erectile dysfunction therapy is most commonly treated by the oral administration of phosphodiesterase-5 (PDE5) inhibitors. Drugs containing active ingredients capable of inhibiting PDE5 such as Viagra® act by increasing the bioavailability of cGMP at the smooth muscle cell level, inhibiting its catabolism mediated by PDE5. As a result the concentration of cGMP in the penile corpus cavernosum is increased and maintained, and the relaxation of the smooth muscles is enhanced, allowing more blood flow to the penis, thereby maintaining the erection. However, these drugs are actually unable to increase nitric oxide synthesis which leads to only short-term improvement of erectile function.

Additionally many patients, and particularly diabetics, don't response to PDE5 inhibition (Hatzimouratidis & Hatzichristou et al., Curr. Pharm. Des. 15(3) 3476-3485 (2009)). In cases where PDE-5 inhibitors are not effective, a second drug, ALPROSTADIL® (Caverjet, Edex, Schwarz Pharma USA Holdings, Inc., Wilmington, Del.) has been shown to be effective. ALPROSTADIL®'s main disadvantage, however, is that it must be injected into the base of the penis into the corpora cavernosa with a needle or inserted into the urethra in pellet form through a delivery system called MUSE (Medicated Urethral Suppository for Erection). Also, an inappropriate dose of either of the afore-mentioned drugs can lead to priapism, or a prolonged erection not due to sexual arousal. Priapism beyond 6 to 8 hours can cause permanent damage to the penis, and requires immediate treatment.

Accordingly there remains a need for new therapies for treating erectile dysfunction, particularly in patients with insulin-dependent diabetes, which suffer from higher rates of sexual dysfunction, and response less favorably to existing medications for treating erectile dysfunction.

C-peptide is the linking peptide between the A- and B-chains in the proinsulin molecule. After cleavage in the endoplasmic reticulum of pancreatic islet β-cells, insulin and a 35 amino acid peptide are generated. The latter is processed to the 31 amino acid peptide, C-peptide, by enzymatic removal of two basic residues on either side of the molecule. C-peptide is co-secreted with insulin in equimolar amounts from the pancreatic islet β-cells into the portal circulation. Besides its contribution to the folding of the two-chain insulin structure, further biologic activity of C-peptide was questioned for many years after its discovery.

Type 1 diabetes, or insulin-dependent diabetes mellitus, is generally characterized by insulin and C-peptide deficiency, due to an autoimmune destruction of the pancreatic islet β-cells. The patients are therefore dependent on exogenous insulin to sustain life. Several factors may be of importance for the pathogenesis of the disease, e.g., genetic background, environmental factors, and an aggressive autoimmune reaction following a temporary infection (Akerblom H K et al.: Annual Medicine 29(5): 383-385, (1997)). Currently insulin-dependent diabetics are provided with exogenous insulin which has been separated from the C-peptide, and thus do not receive exogenous C-peptide therapy. By contrast most type 2 diabetics initially still produce both insulin and C-peptide endogenously, but are generally characterized by insulin resistance in skeletal muscle and adipose tissue.

Type 1 diabetics suffer from a constellation of long-term complications of diabetes that are in many cases more severe and widespread than in type 2 diabetes. Specifically, e.g., microvascular complications involving retina, kidneys, and nerves are a major cause of morbidity and mortality in patients with type 1 diabetes.

There is increasing support for the concept that C-peptide deficiency may play a role in the development of the long-term complications of insulin-dependent diabetics. Additionally, in vivo as well as in vitro studies, in diabetic animal models and in patients with type 1 diabetes, demonstrate that C-peptide possesses hormonal activity (Wahren J et al.: American Journal of Physiology 278: E759-E768, (2000); Wahren J et al.: In International textbook of diabetes mellitus Ferranninni E, Zimmet P, De Fronzo R A, Keen H, Eds. Chichester, John Wiley & Sons, (2004), p. 165-182). Thus, C-peptide used as a complement to regular insulin therapy may provide an effective approach to the management of type 1 diabetes long-term complications.

Studies to date suggest that C-peptide's therapeutic activity involves the binding of C-peptide to a G-protein-coupled membrane receptor, activation of Ca²⁺-dependent intracellular signalling pathways, and phosphorylation of the MAP-kinase system, eliciting increased activities of both sodium/potassium ATPase and endothelial nitrix oxide synthase (eNOS).

Despite these promising in vitro and biochemical studies, and long-felt need for a more effective therapy for the treatment erectile dysfunction in diabetic subjects, C-peptide has yet to be approved for any therapeutic use either for either the treatment of a long term complication of type 1 diabetes, or erectile dysfunction. A significant barrier to the development to a commercially viable C-peptide therapy lies in the need to demonstrate statistically significant effects in the relevant human clinical population under appropriately placebo controlled conditions. Given the high failure rate of existing treatments for erectile dysfunction in the diabetic population, the complexity of the sexual response in humans, and questions as to the degree to which C-peptide can actually prevent or reverse diabetes mediated loss of sexual function in patients with one or more long term complications of type 1 diabetes, the demonstration that C-peptide therapy is actually very effective for treating erectile dysfunction in the patient group represents a major advance in the field.

The present invention is focused on the development of more effective therapies for treating erectile dysfunction. These improved methods for treating erectile dysfunction are based on clinical trial results that surprisingly demonstrate that subcutaneous C-peptide administration results in a significant improvement in sexual function in diabetic patients with insulin-dependent diabetes undergoing C-peptide treatment for the treatment of long term complications of type 1 diabetes.

In one aspect, these therapies are targeted to diabetic patients, and in a further aspect to insulin-dependent patients. In one aspect the insulin-dependent patients are suffering from one or more long term complications of type 1 diabetes. In one aspect of the invention, C-peptide therapy can be combined with a phosphodiesterase inhibitor to provide for a combination therapy with improved therapeutic efficacy compared to the use of a phosphodiesterase inhibitor alone.

### SUMMARY OF THE INVENTION

In one embodiment the present invention includes a method of treating erectile dysfunction in a patient, wherein the patient has insulin-dependent diabetes, comprising the step of administering to the patient in need of such treatment a therapeutic dose of C-peptide.

In another embodiment, the present invention includes the use of C-peptide in the preparation of a medicament for the treatment of erectile dysfunction.

In one aspect of any of these methods the erectile dysfunction includes reduced erection confidence. In another aspect any of these methods the erectile dysfunction includes reduced penetration ability. In one aspect of any of these methods the erectile dysfunction includes reduced erection maintenance or duration. In one aspect of any of these methods the erectile dysfunction includes dysfunction is ejaculation failure.

In another embodiment the present invention includes a method of treating erectile dysfunction in a patient in need thereof, wherein the patient has insulin-dependent diabetes, comprising administering to the patient a therapeutic dose of C-peptide, wherein the C-peptide, enhances relaxation of the penile resistance blood vessels.

In another embodiment the present invention includes a method of treating erectile dysfunction in a patient in need thereof, wherein the patient has insulin-dependent diabetes, comprising administering to the patient a therapeutic dose of C-peptide, wherein the C-peptide, enhances relaxation of human corpus cavernosum and/or corpus spongiosum tissues.

In another embodiment the present invention includes a method of treating erectile dysfunction in a patient in need thereof, wherein the patient has insulin-dependent diabetes, comprising administering to the patient a therapeutic dose of C-peptide, wherein the C-peptide enhances pudendal neuronal activity.

In another embodiment the present invention includes a method of treating erectile dysfunction in a patient in need thereof, wherein the patient has insulin-dependent diabetes, comprising administering to the patient a therapeutic dose of C-peptide, wherein the C-peptide, or a pharmaceutically acceptable salt thereof, enhances erection duration, maintenance or confidence.

In another embodiment the present invention includes a method of treating erectile dysfunction in a patient in need thereof, wherein the patient has insulin-dependent diabetes, comprising administering to the patient a therapeutic dose of C-peptide, wherein the C-peptide, enhances penetration ability.

In one aspect of any of these methods the patient has at least one long term complication of diabetes. In another aspect of any of these methods the patient has peripheral neuropathy. In another aspect of any of these methods the patient has autonomic neuropathy.

In one aspect of any of these methods the C-peptide, relaxes contraction of the human penile resistance blood vessels by at least about 2.5%. In another aspect of any of these methods the C-peptide, enhances relaxation of human corpus cavernosum tissue by at least about 2.5%.

In another aspect of any of these methods the therapeutic dose of C-peptide comprises a daily dose ranging from about 1.5 to about 4.5 mg per 24 hours. In another aspect of any of these methods the therapeutic dose of C-peptide comprises a daily dose ranging from about 0.3 mg to about 1.5 mg per 24 hours. In another aspect of any of these methods, the therapeutic dose of C-peptide comprises a daily dose ranging from about 3.0 mg to about 6 mg per 24 hours. In another aspect of any of these methods, the therapeutic dose of C-peptide maintains an average steady state concentration of C-peptide in the patient's plasma of between about 0.2 nM and about 6 nM.

In another aspect of any of these methods, the therapeutic dose of C-peptide is administered in a single administration. In another aspect of any of these methods, the therapeutic dose of C-peptide is administered in multiple administrations. In another aspect of any of these methods, wherein the therapeutic dose of C-peptide, is administered orally, intravenously, topically, sublingually, or buccally. In another aspect of any of these methods, the therapeutic dose of C-peptide is administered subcutaneously. In another aspect of any of these methods, the therapeutic dose of C-peptide is administered as a sustained release formulation. In another aspect of any of these methods, the C-peptide is PEGylated.

In another embodiment the present invention includes a method of treating erectile dysfunction in a patient, wherein the patient has insulin-dependent diabetes, comprising administering to the patient a therapeutic dose of C-peptide, in combination with a second active agent.

In one aspect of this method, the second active agent is selected from the group consisting of a type V phosphodiesterase inhibitor, apomorphine, testosterone undecanoate, and L-arginine.

In another aspect, of this method, the second active agent is a type V phosphodiesterase (PDE-5) inhibitor. In one aspect of this method, the type V phosphodiesterase inhibitor is selected from the group consisting of sildenafil, tadalafll, vardenafil, zaprinast and pharmaceutically acceptable salts thereof. In one aspect, the type V phosphodiesterase inhibitor is sildenafil, or a pharmaceutically acceptable salt thereof. In another aspect, the type V phosphodiesterase inhibitor is sildenafil citrate. In another aspect, the type V phosphodiesterase inhibitor is tadalafll, or a pharmaceutically acceptable salt thereof. In another aspect, the type V phosphodiesterase inhibitor is vardenafil, or a pharmaceutically acceptable salt thereof. In another aspect, the type V phosphodiesterase 5 inhibitor is zaprinast, or a pharmaceutically acceptable salt thereof. In one aspect of any of these combination therapies, the therapeutic dose of C-peptide, is administered subcutaneously and the type 5 phosphodiesterase (PDE-5) inhibitor is administered orally, intravenously, sublingually, or buccally.

In another aspect of any of these methods, the patient has at least one long term complication of type 1 diabetes. In another aspect, the patient has peripheral neuropathy. In another aspect of any of these methods the patient has autonomic neuropathy.

In another aspect, of any of these methods the therapeutic dose of C-peptide comprises a daily dose ranging from about 1.0 to about 5.0 mg per 24 hours. In another aspect, of any of these methods the therapeutic dose of C-peptide comprises a daily dose ranging from about 1.5 to about 4.5 mg per 24 hours. In another aspect, of any of these methods, the therapeutic dose of C-peptide maintains an average steady state concentration of C-peptide in the patient's plasma of between about 0.2 nM and about 6 nM. In another aspect, of any of these methods, the therapeutic dose of C-peptide is administered as a sustained release formulation. In another aspect of any of these methods, the C-peptide is PEGylated.

In another embodiment the present invention includes a method of treating erectile dysfunction in a patient in need thereof, wherein the patient has insulin-dependent diabetes, comprising administering to the patient a therapeutic dose of C-peptide and a PDE-5 inhibitor, wherein the C-peptide enhances PDE-5 inhibitor induced relaxation of human corpus cavernosum tissue as compared to treatment with a PDE-5 inhibitor alone.

In one aspect of this method, the PDE-5 inhibitor is sildenafil, or a pharmaceutically acceptable salt thereof. In another aspect, the therapeutic dose of C-peptide is administered subcutaneously. In another aspect of any of these methods, the patient has at least one long term complication of type 1 diabetes. In another aspect, the patient has peripheral neuropathy. In another aspect of any of these methods the patient has autonomic neuropathy.

In another aspect of any of these methods, the therapeutic dose of C-peptide comprises a daily dose ranging from about 1.5 mg to about 4.5 mg per 24 hours. In another aspect of any of these methods, the therapeutic dose of C-peptide maintains an average steady state concentration of C-peptide in the patient's plasma of between about 0.2 nM and about 6 nM. In another aspect, of any of these methods, the therapeutic dose of C-peptide is administered as a sustained release formulation. In another aspect of any of these methods, the C-peptide is PEGylated.

In another embodiment the present invention includes a method of treating a erectile dysfunction in a patient in need thereof, wherein the patient has insulin-dependent diabetes, comprising administering to the patient a therapeutic dose of C-peptide, and a PDE-5 inhibitor, wherein the therapeutic dose of C-peptide enhances PDE-5 inhibitor induced dilation of human penile resistance blood vessels compared to the dilation level that occurs with PDE-5 inhibitor administration alone.

In one aspect of this method, the PDE-5 inhibitor is sildenafil, or a pharmaceutically acceptable salt thereof. In another aspect, the therapeutic dose of C-peptide is administered subcutaneously. In another aspect of any of these methods, the patient has at least one long term complication of type 1 diabetes. In another aspect, the patient has peripheral neuropathy.

In another aspect of any of these methods, the therapeutic dose of C-peptide comprises a daily dose ranging from about 1.5 mg to about 4.5 mg per 24 hours. In another aspect of any of these methods, the therapeutic dose of C-peptide maintains an average steady state concentration of C-peptide in the patient's plasma of between about 0.2 nM and about 6 nM. In another aspect, of any of these methods, the therapeutic dose of C-peptide is administered as a sustained release formulation. In another aspect of any of these methods, the C-peptide is PEGylated.

In another embodiment the present invention includes a method of enhancing PDE-5 inhibitor-induced relaxation of human corpus cavernosum tissue in a patient receiving a PDE-5 inhibitor, wherein the patient has diabetes, comprising administering to the patient a therapeutic dose of C-peptide, wherein PDE-5 inhibitor-induced relaxation of human corpus cavernosum tissue is enhanced compared to treatment with a PDE-5 inhibitor alone.

In one aspect of this method, the PDE-5 inhibitor is sildenafil, or a pharmaceutically acceptable salt thereof. In another aspect, the therapeutic dose of C-peptide is administered subcutaneously. In another aspect of any of these methods, the patient has insulin dependent diabetes. In another aspect of any of these methods, the patient has at least one long term complication of diabetes. In another aspect, the patient has peripheral neuropathy. In another aspect of any of these methods the patient has autonomic neuropathy.

In another aspect of any of these methods, the therapeutic dose of C-peptide comprises a daily dose ranging from about 1.5 mg to about 4.5 mg per 24 hours. In another aspect of any of these methods, the therapeutic dose of C-peptide maintains an average steady state concentration of C-peptide in the patient's plasma of between about 0.2 nM and about 6 nM. In another aspect, of any of these methods, the therapeutic dose of C-peptide is administered as a sustained release formulation. In another aspect of any of these methods, the C-peptide is PEGylated.

In another embodiment the current invention includes a method of enhancing PDE-5 inhibitor-mediated dilation of human penile resistance blood vessels in a patient receiving a PDE-5 inhibitor, wherein the patient has diabetes, comprising administering to the patient a therapeutic dose of C-peptide, wherein dilation of the human penile resistance blood vessels is enhanced as compared to the dilation level that occurs with PDE-5 inhibitor administration alone.

In one aspect of this method, the PDE-5 inhibitor is sildenafil, or a pharmaceutically acceptable salt thereof. In another aspect, the therapeutic dose of C-peptide is administered subcutaneously. In another aspect of any of these methods, the patient has insulin dependent diabetes. In another aspect of any of these methods, the patient has at least one long term complication of diabetes. In another aspect, the patient has peripheral neuropathy. In another aspect of any of these methods the patient has autonomic neuropathy.

In another aspect of any of these methods, the therapeutic dose of C-peptide comprises a daily dose ranging from about 1.5 mg to about 4.5 mg per 24 hours. In another aspect of any of these methods, the therapeutic dose of C-peptide maintains an average steady state concentration of C-peptide in the patient's plasma of between about 0.2 nM and about 6 nM. In another aspect, of any of these methods, the therapeutic dose of C-peptide is administered as a sustained release composition. In another aspect of any of these methods, the C-peptide is PEGylated.

In another embodiment, the current invention includes the use of C-peptide in the preparation of a medicament for the treatment of erectile dysfunction in a human patient.

In another embodiment, the current invention includes the use of C-peptide for the treatment of erectile dysfunction in a human patient with diabetes, wherein said C-peptide is administered in a regimen which maintains an average steady state concentration of C-peptide in said patient's plasma above about 0.2 nM.

In one aspect of either of these uses, the patient has insulin dependent diabetes. In another aspect of any of these uses, the patient has at least one long term complication of diabetes. In another aspect of any of these uses, patient has peripheral neuropathy. In another aspect of any of these uses the patient has autonomic neuropathy.

In another aspect of any of these uses, C-peptide is administered as a daily therapeutic dose ranging from about 1.5 to about 4.5 mg per 24 hours. In another aspect of any of these uses, therapeutic dose of C-peptide maintains an average steady state concentration of C-peptide in said patient's plasma of between about 0.2 nM and about 6 nM. In another aspect of any of these uses, C-peptide is administered as a sustained release composition. In another aspect of any of these uses, the C-peptide is PEGylated.

### BRIEF DESCRIPTION OF THE DRAWINGS

A better understanding of the features and advantages of the present invention can be obtained by reference to the following detailed description that sets forth illustrative embodiments, in which the principles of the invention are utilized, and the accompanying drawings of which:

FIG. 1 shows an overview flow chart of visits and variables determined during the clinical trial of C-peptide therapy (as more fully described in Examples).

FIG. 2 shows the disposition of patients in the study (ITT=intend-to-treat; PP=per-protocol).

FIG. 3 shows C-peptide plasma levels in the low- and high-dose groups at the 3 month visit (diamond symbols) in relation to theoretical pharmacokinetic data (solid line) extrapolated from an earlier study.

FIG. 4 shows the change in peak sensory nerve conduction velocity (SCVp) from baseline to 6 months of treatment in patients with SCVp>−2.5 standard deviations (SD) at baseline. Active represents combination of low- and high-dose C-peptide groups.

FIG. 5 shows the median changes in perception thresholds and neurological impairment assessment scores in patients from baseline to 6 months of treatment. Active represents combination of low- and high-dose C-peptide groups.

### DETAILED DESCRIPTION OF THE INVENTION

### Definitions

In order that the present disclosure may be more readily understood, certain terms are first defined. Additional definitions are set forth throughout the detailed description.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. As used herein, the term “increase” or the related terms “increased”, “enhance” or “enhanced” refers to a statistically significant increase. For the avoidance of doubt, the terms generally refer to at least a 2%, at least about 5%, at least about 10% increase in a given parameter, and can encompass at least 20%, 50%, 75%, 100%, 150% or more.

As used herein and in the appended claims, the singular forms “a”, “an”, and “the” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

The term “Cₘₐₓ” as used herein is the maximum serum or plasma concentration of drug which occurs during the period of release which is monitored.

The term “Cₘᵢₙ” as used herein is the minimum serum or plasma concentration of drug which occurs during the period of release during the treatment period.

The term “Cₐᵥₑ” as used herein is the average serum concentration of drug derived by dividing the area under the curve (AUC) of the release profile by the duration of the release.

The term “Cₛₛ₋ₐᵥₑ” as used herein is the average steady-state concentration of drug obtained during a multiple dosing regimen after dosing for at least five elimination half-lives. It will be appreciated that drug concentrations are fluctuating within dosing intervals even once an average steady state concentration of drug has been obtained.

The term “tₘₐₓ” as used herein is the time post-dose at which Cₘₐₓ is observed.

The term “AUC” as used herein means “area under curve” for the serum or plasma concentration-time curve, as calculated by the trapezoidal rule over the complete sample collection interval.

The term “bioavailability” refers to the amount of drug that reaches the circulation system expressed in percent of that administered. The amount of bioavailable material can be defined as the calculated AUC for the release profile of C-peptide during the time period starting at post-administration and ending at a predetermined time point. As is understood in the art, a release profile is generated by graphing the serum levels of a biologically active agent in a subject (Y-axis) at predetermined time points (X-axis). Bioavailability is often referred to in terms of bioavailability, which is the bioavailability achieved for a drug (such as C-peptide) following administration of a sustained release composition of that drug divided by the bioavailability achieved for the drug following intravenous administration of the same dose of drug, multiplied by 100.

The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz G E and R H Schirmer, Principles of Protein Structure, Springer-Verlag (1979)). According to such analyses, groups of amino acids can be defined where amino acids within a group exchange preferentially with each other, and therefore resemble each other most in their impact on the overall protein structure (Schulz G E and R H Schirmer, Principles of Protein Structure, Springer-Verlag (1979)).

Examples of amino acid groups defined in this manner include: a “charged/polar group,” consisting of Glu, Asp, Asn, Gln, Lys, Arg, and His; an “aromatic or cyclic group,” consisting of Pro, Phe, Tyr, and Trp; and an “aliphatic group,” consisting of Gly, Ala, Val, Leu, Ile, Met, Ser, Thr, and Cys.

Within each group, subgroups can also be identified, e.g., the group of charged/polar amino acids can be sub-divided into the subgroups consisting of the “positively-charged subgroup,” consisting of Lys, Arg, and His; the “negatively-charged subgroup,” consisting of Glu and Asp, and the “polar subgroup” consisting of Asn and Gln. The aromatic or cyclic group can be sub-divided into the subgroups consisting of the “nitrogen ring subgroup,” consisting of Pro, His, and Trp; and the “phenyl subgroup” consisting of Phe and Tyr. The aliphatic group can be sub-divided into the subgroups consisting of the “large aliphatic non-polar subgroup,” consisting of Val, Leu, and Ile; the “aliphatic slightly-polar subgroup,” consisting of Met, Ser, Thr, and Cys; and the “small-residue sub-group,” consisting of Gly and Ala.

Examples of conservative mutations include amino acid substitutions of amino acids within the subgroups above, e.g., Lys for Arg and vice versa such that a positive charge can be maintained; Glu for Asp and vice versa such that a negative charge can be maintained; Ser for Thr such that a free —OH can be maintained; and Gln for Asn such that a free —NH₂ can be maintained. “Semi-conservative mutations” include amino acid substitutions of amino acids with the same groups listed above, that do not share the same subgroup. For example, the mutation of Asp for Asn, or Asn for Lys, all involve amino acids within the same group, but different subgroups. “Non-conservative mutations” involve amino acid substitutions between different groups, e.g., Lys for Leu, or Phe for Ser, etc.

The terms “diabetes”, “diabetes mellitus”, or “diabetic condition”, unless specifically designated otherwise, encompass all forms of diabetes. The term “Type 1 diabetic” or “Type 1 diabetes” refers to a patient with a fasting plasma glucose concentration of greater than about 7.0 mmoL/L and a fasting C-peptide level of about, or less than about 0.2 nmoL/L. The term “Type 1.5 diabetic” or “Type 1.5 diabetes” refers to a patient with a fasting plasma glucose concentration of greater than about 7.0 mmoL/L and a fasting C-peptide level of about, or less than about 0.4 nmoL/L. The term “Type 2 diabetic” or “Type 2 diabetes” generally refers to a patient with a fasting plasma glucose concentration of greater than about 7.0 mmoL/L and fasting C-peptide level that is within or higher than the normal physiological range of C-peptide levels (about 0.47 to 2.5 nmoL/L). It will be appreciated that a patient initially diagnosed as a type 2 diabetic may subsequently develop insulin-dependent diabetes, and may remain diagnosed as a type 2 patient, even though their C-peptide levels drop to those of a type 1.5 or type 1 diabetic patient (<0.2 nM).

The term “delivery agent” refers to carrier compounds or carrier molecules that are effective in the oral delivery of therapeutic agents, and may be used interchangeably with “carrier”.

As used herein, the term “erectile dysfunction” or “ED” refers a periodic or consistent inability to achieve or sustain an erection of sufficient rigidity for sexual intercourse, including reduced erection duration, maintenance, confidence or lack of penetration ability.

As used herein, the term “ejaculatory dysfunction” refers to all forms of ejaculatory dysfunction including, ejaculation failure, retarded ejaculation, retrograde ejaculation, anejaculation, aspermia, haemospermia, low volume ejaculate, painful ejaculation and anhedonia (i.e., lack of pleasure)

The term “homology” describes a mathematically-based comparison of sequence similarities which is used to identify genes or proteins with similar functions or motifs. The nucleic acid and protein sequences of the present invention can be used as a “query sequence” to perform a search against public databases to, e.g., identify other family members, related sequences, or homologs. Such searches can be performed using the NBLAST and XBLAST programs (version 2.0) of Altschul et al.: J. Mol. Biol. 215: 403-410, (1990)). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al.: Nucleic Acids Res. 25(17): 3389-3402, (1997). When utilizing BLAST and Gapped BLAST programs, the default parameters of the respective programs (e.g., XBLAST and BLAST) can be used (see www.ncbi.nlm.nih.gov).

The term “homologous” refers to the relationship between two proteins that possess a “common evolutionary origin”, including proteins from superfamilies (e.g., the immunoglobulin superfamily) in the same species of animal, as well as homologous proteins from different species of animal (e.g., myosin light chain polypeptide, etc.; see Reeck et al.: Cell 50: 667, (1987)). Such proteins (and their encoding nucleic acids) have sequence homology, as reflected by their sequence similarity, whether in terms of percent identity or by the presence of specific residues or motifs and conserved positions. In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, e.g., the GCG (Genetics Computer Group, version 7, Madison, Wis.) pileup program, or using any of the programs and algorithms described above. The program may use the local homology algorithm of Smith and Waterman with the default values: gap creation penalty=−(1+1/3 k), k being the gap extension number, average match=1, average mismatch=−0.333.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk A M, ed., Oxford University Press, New York, (1988); Biocomputing: Informatics and Genome Projects, Smith D W, ed., Academic Press, New York, (1993); Computer Analysis of Sequence Data, Part I, Griffin A M and Griffin H G, eds., Humana Press, New Jersey, (1994); Sequence Analysis in Molecular Biology, von Heinje G, Academic Press, (1987); and Sequence Analysis Primer, Gribskov M and Devereux J, eds., M Stockton Press, New York, (1991); and Carillo H and Lipman D, SIAM J. Applied Math., 48: 1073, (1988)). Methods to determine identity are designed to give the largest match between the sequences tested. Moreover, methods to determine identity are codified in publicly available computer programs. Computer program methods to determine identity between two sequences include, but are not limited to, the GCG program package (Devereux J et al.: Nucleic Acids Res. 12(1): 387, (1984)), BLASTP, BLASTN, and FASTA (Altschul S F et al.: J. Molec. Biol. 215: 403-410, (1990) and Altschul S F et al.: Nucleic Acids Res. 25: 3389-3402, (1997)). The BLAST X program is publicly available from NCBI and other sources (BLAST Manual, Altschul S F et al., NCBI NLM NIH Bethesda, Md. 20894; Altschul S F et al., J. Mol. Biol. 215: 403-410, (1990)). The well-known Smith Waterman algorithm (Smith T F, Waterman M S: J. Mol. Biol. 147(1): 195-197, (1981)) can also be used to determine similarity between sequences.

The term “insulin” includes all forms of insulin including, without limitation, rapid-acting forms, such as Insulin Lispro rDNA origin: HUMALOG (1.5 mL, 10 mL, Eli Lilly and Company, Indianapolis, Ind.), Insulin Injection (Regular Insulin) from beef and pork (regular ILETIN I, Eli Lilly), human: rDNA: HUMULIN R (Eli Lilly), NOVOLIN R (Novo Nordisk, New York, N.Y.), Semi synthetic: VELOSULIN Human (Novo Nordisk), rDNA Human, Buffered: VELOSULIN BR, pork: regular Insulin (Novo Nordisk), purified pork: Pork Regular ILETIN II (Eli Lilly), Regular Purified Pork Insulin (Novo Nordisk), and Regular (Concentrated) ILETIN II U-500 (500 units/mL, Eli Lilly); intermediate-acting forms such as Insulin Zinc Suspension, beef and pork: LENTE ILETIN G I (Eli Lilly), Human, rDNA: HUMULIN L (Eli Lilly), NOVOLIN L (Novo Nordisk), purified pork: LENTE ILETIN II (Eli Lilly), Isophane Insulin Suspension (NPH): beef and pork: NPH ILETIN I (Eli Lilly), Human, rDNA: HUMULIN N (Eli Lilly), Novolin N (Novo Nordisk), purified pork: Pork NPH Eetin II (Eli Lilly), NPH-N (Novo Nordisk); and long-acting forms such as Insulin zinc suspension, extended (ULTRALENTE, Eli Lilly), human, rDNA: HUMULIN U (Eli Lilly).

The terms “insulin-dependent patient” or “insulin-dependent diabetes” encompass all forms of diabetics/diabetes who/that require insulin administration to adequately maintain normal glucose levels unless specifically specified otherwise. Diabetes is frequently diagnosed by measuring fasting blood glucose, insulin, or glycated hemoglobin levels (which are typically referred to as hemoglobin A1c, Hb_{1c}, Hb_{A1c}, or A1C). Normal adult glucose levels are 60-126 mg/dL. Normal insulin levels are 30-60 pmol/L. Normal HbA1c levels are generally less than 6%. The World Health Organization defines the diagnostic value of fasting plasma glucose concentration to 7.0 mmoL/L (126 mg/dL) and above for diabetes mellitus (whole blood 6.1 mmoL/L or 110 mg/dL), or 2-hour glucose level greater than or equal to 11.1 mmoL/L (greater than or equal to 200 mg/dL). Other values suggestive of or indicating high risk for diabetes mellitus include elevated arterial pressure greater than or equal to 140/90 mm Hg; elevated plasma triglycerides (greater than or equal to 1.7 mmoL/L [150 mg/dL]) and/or low HDL-cholesterol (less than 0.9 mmoL/L [35 mg/dL] for men; and less than 1.0 mmoL/L [39 mg/dL] for women); central obesity (BMI exceeding 30 kg/m²); microalbuminuria, where the urinary albumin excretion rate is greater than or equal to 20 μg/min or the albumin creatinine ratio is greater than or equal to 30 mg/g.

The term “multiple dose” means that the patient has received at least two doses of the drug composition in accordance with the dosing interval for that composition.

The term “normal glucose levels” is used interchangeably with the term “normoglycemic” and “normal” and refers to a fasting venous plasma glucose concentration of less than about 6.1 mmoL/L (110 mg/dL). Sustained glucose levels above normoglycemic are considered a pre-diabetic condition.

As used herein, the term “patient” in the context of the present invention is preferably a mammal. The mammal can be a human, non-human primate, mouse, rat, dog, cat, horse, or cow, but are not limited to these examples. Mammals other than humans can be advantageously used as patients that represent animal models of insulin-dependent diabetes mellitus, or diabetic conditions. A patient can be male. A patient can be one who has been previously diagnosed or identified as having insulin-dependent diabetes, or a diabetic condition, and optionally has already undergone, or is undergoing, a therapeutic intervention for the diabetes. A patient can also be one who is suffering from a long-term complication of type 1 diabetes.

The term “PDE inhibitors” as used herein, is intended to include, both selective and non selective inhibitors of type 5 cGMP-specific phosphodiesterase. Sources of information for the above, and other, phosphodiesterase inhibitors include Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), the disclosures of each of which are incorporated herein by reference in their entirety. The PDE inhibitor specificity can also be determined by standard assays known to the art, for example as disclosed in U.S. Pat. No. 5,250,534, incorporated herein by reference. Compounds which are selective inhibitors of cGMP PDE relative to cAMP PDE are preferred, and determination of such compounds is also taught in U.S. Pat. No. 5,250,534. Particularly preferred are compounds which selectively inhibit the PDE V enzyme, as disclosed in WO 94/28902. The terms “phosphodiesterase 5 inhibitors”, “PDE-5 inhibitors” or “PDE5 inhibitors” refer to selective inhibitors of cGMP-specific phosphodiesterase V.

In one aspect, PDE-5 inhibitors are selected from the group of PDE-5 Inhibitors consisting of Tadalafil ((6R,12aR)-2,3,6,7,12,12a-Hexahydro-2-methyl-6-(3,4-methylene-dioxyphenyl) pyrazino(1′,2′: 1,6) pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-1-9-propyl-2,4,7,8-tetrazabicyclo[4.3.0]nona-3,8,10-trien-5-one), Udenafil 5-[2-propyloxy-5-(1-methyl-2-pyrrolidinyl-ethyl-amidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxy phenyl)methyl]amino}-2-[(2S)₂-(hydroxymethyl)pyrrolidin-1-yl]-N-(pyrimidin-2-ylmethyl)pyrimidine-5-carboxamide, SLx 2101 of Surface Logix, LAS 34179Triazolo[1,2]xanthine, 6-methyl-4-propyl-2-[2-propoxy-5-(4-methylpiperazino)sulfonyl]phenyl, deuterated and/or ¹³C-containing isotopologues, or pharmaceutically acceptable salts, hydrates or hydrates of salts thereof.

The term “rapid release” refers to the release of a drug such as C-peptide from a rapid release formulation or rapid release device which occurs over a period which is shorter than that period during which the C-peptide would be available following direct S.C. administration of a single dose of C-peptide.

The term “replacement dose” in the context of a replacement therapy for C-peptide refers to a dose of C-peptide that maintains C-peptide levels in the blood within a desirable range, particularly at a level which is at or above the minimum effective therapeutic level. In another aspect, the replacement dose maintains the average steady-state concentration C-peptide levels above a minimum level of about 0.1 nM between dosing intervals. In a preferred aspect the replacement dose maintains the average steady state concentration C-peptide levels above a minimum level of about 0.2 nM between dosing intervals.

The term “Standard Deviation Score” or “SDS”, when referring to nerve conduction velocity, refers to the observed value minus the mean of the reference value divided by the Standard Deviation of the method. The quantitative sensory testing (QST) data are presented as corrected for age. The reference values were estimated from linear regression analysis of data in a cohort of 63 healthy subjects (27 men and 36 women, 22-55 years of age, body height 150-196 cm).

The terms “subcutaneous” or “subcutaneously” or “S.C.” in reference to a mode of administration of insulin or C-peptide, refers to a drug that is administered as a bolus injection, or via an implantable device into the area in, or below the subcutis, the layer of skin directly below the dermis and epidermis, collectively referred to as the cutis. Preferred sites for subcutaneous administration and/or implantation include the outer area of the upper arm, just above and below the waist, except the area right around the navel (a 2-inch circle). The upper area of the buttock, just behind the hipbone. The front of the thigh, midway to the outer side, 4 inches below the top of the thigh to 4 inches above the knee.

The term “single dose” means that the patient has received a single dose of the drug composition or that the repeated single doses have been administered with washout periods in between. Unless specifically designated as “single dose” or at “steady-state” the pharmacokinetic parameters disclosed and claimed herein encompass both single-dose and multiple-dose conditions.

The term “sequence similarity” refers to the degree of identity or correspondence between nucleic acid or amino acid sequences that may or may not share a common evolutionary origin (see Reeck et al., supra). However, in common usage and in the present application, the term “homologous”, when modified with an adverb such as “highly”, may refer to sequence similarity and may or may not relate to a common evolutionary origin.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

As defined herein, the terms “sustained release”, “extended release”, or “depot formulation” refers to the release of a drug such as C-peptide from the sustained release composition or sustained release device which occurs over a period which is longer than that period during which the C-peptide would be available following direct I.V. or S.C. administration of a single dose of C-peptide. In one aspect, sustained release will be a release that occurs over a period of at least about one to two weeks. In another aspect, sustained release will be a release that occurs over a period of at least about one year. The continuity of release and level of release can be affected by the type of sustained release device (e.g., programmable pump or osmotically-driven pump) or sustained release composition used (e.g., monomer ratios, molecular weight, block composition, and varying combinations of polymers), degree or size of the PEGylating moiety, polypeptide loading, and/or selection of excipients to produce the desired effect, as more fully described herein.

Various sustained release profiles can be provided in accordance with any of the methods of the present invention. “Sustained release profile” means a release profile in which less than 50% of the total release of C-peptide that occurs over the course of implantation/insertion or other method of administering C-peptide in the body occurs within the first 24 hours of administration. In a preferred embodiment of the present invention, the extended release profile is selected from the group consisting of; a) the 50% release point occurring at a time that is between 48 and 72 hours after implantation/insertion or other method of administration; b) the 50% release point occurring at a time that is between 72 and 96 hours after implantation/insertion or other method of administration; c) the 50% release point occurring at a time that is between 96 and 110 hours after implantation/insertion or other method of administration; d) the 50% release point occurring at a time that is between 1 and 2 weeks after implantation/insertion or other method of administration; e) the 50% release point occurring at a time that is between 2 and 4 weeks after implantation/insertion or other method of administration; f) the 50% release point occurring at a time that is between 4 and 8 weeks after implantation/insertion or other method of administration; g) the 50% release point occurring at a time that is between 8 and 16 weeks after implantation/insertion or other method of administration; h) the 50% release point occurring at a time that is between 16 and 52 weeks (1 year) after implantation/insertion or other method of administration; and i) the 50% release point occurring at a time that is between 52 and 104 weeks after implantation/insertion or other method of administration.

Additionally, use of a sustained release composition can reduce the degree of fluctuation (“DFL”) of C-peptide's plasma concentration. DFL is a measurement of how much the plasma levels of a drug vary over the course of a dosing interval (Cₘₐₓ−Cₘᵢₙ/Cₘᵢₙ). For simple cases, such as I.V. administration, fluctuation is determined by the relationship between the elimination half-life (t_{1/2}) and dosing interval. If the dosing interval is equal to the half-life then the trough concentration is exactly half of the peak concentration, and the degree of fluctuation is 100%. Thus a sustained release composition with a reduced DFL (for the same dosing interval) signifies that the difference in peak and trough plasma levels has been reduced. Preferably, the patients receiving a sustained release composition of C-peptide have a DFL approximately 50%, 40%, or 30% of the DFL in patients receiving a non-extended release composition with the same dosing interval.

The terms “treating” or “treatment” means to relieve, alleviate, delay, reduce, reverse, improve, manage, or prevent at least one symptom of a condition in a patient. The term “treating” may also mean to arrest, delay the onset (i.e., the period prior to clinical manifestation of a disease), and/or reduce the risk of developing or worsening a condition.

As used herein, the terms “therapeutically effective amount”, “therapeutic dose”, “prophylactically effective amount”, or “diagnostically effective amount” is the amount of the drug, e.g., insulin or C-peptide, needed to elicit the desired biological response following administration. Similarly the term “C-peptide therapy” refers to a therapy that maintains the average steady state concentration C-peptide in the patient's plasma above the minimum effective therapeutic level.

The term “Unit-Dose Forms” refers to physically discrete units suitable for human and animal patients and packaged individually as is known in the art. It is contemplated for purposes of the present invention that dosage forms of the present invention comprising therapeutically effective amounts of C-peptide may include one or more unit doses (e.g., tablets, capsules, powders, semisolids [e.g., gelcaps or films], liquids for oral administration, ampoules or vials for injection, loaded syringes) to achieve the therapeutic effect. It is further contemplated for the purposes of the present invention that a preferred embodiment of the dosage form is a subcutaneously injectable dosage form.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references

The publications discussed above are provided solely for their disclosure before the filing date of the present application. Nothing herein is to be construed as an admission that the invention is not entitled to antedate such disclosure by virtue of prior invention.

Overview of Methods for Treating Erectile Dysfunction

The present invention relates to the development of improved methods for treating sexual dysfunction associated with diabetes, and in one aspect with insulin-dependent diabetes. Significantly, such dosing regimens can be combined with established methods for treating erectile dysfunction, including PDE5 inhibitors sold under the trademark VIAGRA® to provide for significantly improved efficacy compared to the PDE5 inhibitor alone.

In one embodiment, the present invention includes a method of treating sexual dysfunction in a patient, comprising the step of administering to the patient in need of such treatment a therapeutic dose of C-peptide.

In another aspect, the present invention includes a, method of treating erectile dysfunction in a patient in need thereof comprising administering to the patient a therapeutic dose of C-peptide.

In another aspect, the present invention includes a method of enhancing sexual desire, and sexual satisfaction in a patient in need thereof comprising administering to the patient a therapeutic dose of C-peptide.

In another embodiment, the present invention includes a method of treating sexual dysfunction in a patient comprising administering to the patient a therapeutic dose of C-peptide, in combination with a second active agent.

In another aspect, the present includes a method of treating erectile dysfunction in a patient in need thereof comprising administering to said patient a therapeutic dose of C-peptide and a PDE-5 inhibitor.

Male Sexual Dysfunction

The male erectile response is initiated by neuronal activity and is maintained by a complex interplay between events involving blood vessels (i.e., vascular events) and events involving the nervous system (i.e., neurological events).

It is parasympathetic neuronal action that initiates the male erectile response. Specifically, this parasympathetic input originates from the pelvic splanchnic nerve plexus (pudendal nerve). The pelvic splanchnic nerve plexus is comprised of branches from the second, third, and fourth sacral nerves that intertwine with the inferior hypogastric plexus, which is a network of nerves in the pelvis. The cavernous nerves are derived from the pelvic splanchnic nerves, via the prostatic plexus, and supply parasympathetic fibers to the corpora cavernosa and corpus spongiosum, the spongy tissues in the penis that are engorged with blood during an erection.

The corpora cavernosa are two paired tissue bodies that lie dorsally in the penis, while the corpus spongiosum is located ventrally and surrounds the urethra. The corpus spongiosum expands at the terminal end to form the glans penis. These erectile tissues are comprised of venous spaces lined with epithelial cells separated by connective tissue and smooth muscle cells.

Parasympathetic stimulation of the autonomic nervous system allows erection by relaxation of the smooth muscle and dilation of penile resistance vessels including the helicine arteries, which are arteries found in the erectile tissue of the penis. Dilation is caused by the vasodilatory effects of cGMP, the production of which is stimulated by the release of nitric oxide (NO). NO release in the corpus cavernosum is induced by neuronal impulses derived from parasympathetic neuronal stimulation. The dilation of the arteries causes greatly increased blood flow through the erectile tissue, which leads to expansion of the corpora cavernosa and the corpus spongiosum. As the corpora cavernosa and the corpus spongiosum expand, the venous structures draining the penis are compressed against the fascia surrounding each of the erectile tissues. Thus, the outflow of blood is restricted and the internal pressure increases. This vein-obstruction process is referred to as the corporal veno-occlusive mechanism.

Conversely, sympathetic innervation from the hypogastric nerves and/or certain nerves of the inferior hypogastric plexus, which derive from the sympathetic ganglia, inhibit parasympathetic activity and cause constriction of the smooth muscle and helicine arteries, making the penis flaccid. The flaccid state is maintained by continuous sympathetic (alpha-adrenergic) nervous system stimulation of the penile blood vessels and smooth muscle.

Accordingly in one aspect the present invention includes a method of treating erectile dysfunction in a patient in need thereof comprising administering to the patient a therapeutic dose of C-peptide, wherein said C-peptide, enhances pudendal nerve activity. In one aspect of this method, C-peptide treatment results in about 2%, about 5%, about 10%, about 15%, about 20%, about 25%, about 30% or about 35 increase in pudendal neuronal activity, compared to the maximum parasympathetic neuronal activity measured before starting C-peptide therapy.

In another aspect the present invention includes a method of treating erectile dysfunction in a patient in need thereof comprising administering to the patient a therapeutic dose of C-peptide, wherein said C-peptide, enhances the dilation of the penile resistance vessels. In one aspect of this method, the C-peptide treatment results in about 2%, about 5%, about 10%, about 15%, about 20%, about 25%, about 30% or about 35% increase in dilation of the penile resistance vessels, compared to the maximum dilation of the penile resistance vessels measured before starting C-peptide therapy.

In another aspect, the present invention includes a method of treating erectile dysfunction in a patient in need thereof comprising administering to the patient a therapeutic dose of C-peptide, wherein the C-peptide, enhances relaxation of human corpus cavernosum and/or corpus spongiosum tissues. In one aspect of this method, the C-peptide treatment results in about 2%, about 5%, about 10%, about 15%, about 20%, about 25%, about 30% or about 35% increase in relaxation of human corpus cavernosum and/or the corpus spongiosum tissues, compared to the maximum relaxation of human corpus cavernosum and/or the corpus spongiosum tissues measured before starting C-peptide therapy.

In another aspect, the present invention includes a method of treating erectile dysfunction in a patient in need thereof comprising administering to the patient a therapeutic dose of C-peptide, wherein the C-peptide, enhances erection duration, maintenance, confidence or enhances penetration ability. In one aspect of this method, the C-peptide treatment results in about 5%, about 10%, about 15%, about 20%, about 25%, about 30% or about 35% increase in self reported score in any criteria of a questionnaire intended to assess in whole or part erection quality. In one aspect, the questionnaire is based in whole or part on the International Index of Erectile Function.

In another embodiment, the invention includes a method of treating a erectile dysfunction in a patient in need thereof comprising administering to said patient a therapeutic dose of C-peptide and a PDE-5 inhibitor, wherein the C-peptide enhances PDE-5 inhibitor induced relaxation of human corpus cavernosum tissue as compared to treatment with a PDE-5 inhibitor alone.

In one aspect of this method, the C-peptide treatment results in about 2%, about 5%, about 10%, about 15%, about 20%, about 25%, about 30% or about 35 increase in relaxation of human corpus cavernosum tissues, compared to the maximum relaxation of human corpus cavernosum tissues measured with the PDE-5 inhibitor before starting C-peptide therapy.

In another aspect, the present invention includes a method of treating a erectile dysfunction in a patient in need thereof comprising administering to the patient a therapeutic dose of C-peptide, and a PDE-5 inhibitor, wherein the therapeutic dose of C-peptide enhances PDE-5 inhibitor induced dilation of penile resistance vessels compared to the dilation level that occurs with PDE-5 inhibitor administration alone.

In one aspect of this method, the C-peptide treatment results in about 2%, about 5%, about 10%, about 15%, about 20%, about 25%, about 30% or about 35 increase in dilation of the penile resistance vessels, compared to the maximum dilation of the penile resistance vessels measured with the PDE-5 inhibitor before starting C-peptide therapy, before starting C-peptide therapy.

The administration of C-peptide to treat erectile dysfunction has particular relevance when administered in combination with a PDE-5 inhibitor to a patient who continues to have symptoms of sexual dysfunction despite treatment with a PDE-5 inhibitor.

In another embodiment the present invention includes a method of enhancing PDE-5 inhibitor-induced relaxation of human corpus cavernosum tissue in a patient receiving a PDE-5_inhibitor, comprising administering to the patient a therapeutic dose of C-peptide, wherein PDE-5 inhibitor-induced relaxation of human corpus cavernosum tissue is enhanced compared to treatment with a PDE-5 inhibitor alone. In one aspect of this method, the C-peptide treatment results in about 2%, about 5%, about 10%, about 15%, about 20%, about 25%, about 30% or about 35% increase in relaxation of human corpus cavernosum tissues, compared to the maximum relaxation of human corpus cavernosum tissues measured with the PDE-5 inhibitor before starting C-peptide therapy.

In another aspect, the present invention includes a method of enhancing PDE-5 inhibitor-mediated dilation of helicine arteries in a patient receiving a PDE-5 inhibitor comprising administering to the patient a therapeutic dose of C-peptide, wherein dilation of the penile resistance vessels is enhanced as compared to the dilation level that occurs with PDE-5 inhibitor administration alone. In one aspect of this method, the C-peptide treatment results in about 2%, about 5%, about 10%, about 15%, about 20%, about 25%, about 30% or about 35% increase in dilation of the penile resistance vessels compared to the maximum dilation of the penile resistance vessels measured with the PDE-5 inhibitor before starting C-peptide therapy, before starting C-peptide therapy.

In one aspect of any of these methods the patient receiving a PDE-5 inhibitor is unresponsive to the PDE-5 inhibitor. Clinically, a patient is sub-optimally responsive to treatment with a PDE-5 inhibitor when the patient scores 21 or less (corresponding to a disease severity of mild-to-moderate or worse) on the IIEF Erectile Function domain despite PDE-5 inhibitor treatment. In general, a patient is sub-optimally responsive to PDE-5 inhibitor treatment when the subject attempts and fails to complete sexual intercourse over the course of several weeks, while being treated with a PDE-5 inhibitor.

In any of the claimed methods C-peptide may be administered as a daily replacement dose. In another aspect of any of the claimed methods C-peptide therapy may be administered for at least about one week, at least about two weeks, at least about three weeks, at least about four weeks, at least about two months or at least about three months.

In one aspect of any of the claimed methods, the therapeutic dose of C-peptide maintains an average steady state concentration of C-peptide in said patient's plasma of between about 0.2 nM and about 6 nM. In one aspect, of any of these methods, the therapeutic dose of C-peptide is administered as a sustained release formulation. In another aspect of any of the claimed methods, the therapeutic dose of C-peptide is administered as a rapid release formulation. In another aspect of any of the claimed methods, the therapeutic dose of C-peptide is administered via S.C. injection.

In any of these methods, various approaches can be used to assess the severity of sexual dysfunction, and the effect of treatments, including for example, direct measurement of penile erection strength (e.g., nocturnal tumescence and rigidity values) and frequency of erection, e.g. using devices such as RigiScan (Timm Medical Technologies, Eden, Prairie, Minn., USA).

Additionally assessments of sexual dysfunction can be completed using self-report techniques. This approach is sometimes considered more satisfactory than direct measurement of penile erection strength in men (Lowy et al., J. Sex. Med. 4(1) 83-92 (2007)). For men the “International Index of Erectile Function” (IIEF) was developed. It can assess five modalities of sexual function: erectile function, orgasmic function, sexual desire, intercourse satisfaction, and overall satisfaction. A reduced set of IIEF, called IIEF-5, and similar Quality of Erection Questionnaire (QEQ) are widely used to assess of erectile dysfunction.

Insulin-Dependent Diabetes

In one aspect of any of the methods disclosed herein, the term “patient” refers to a patient with insulin-dependent diabetes. The terms “insulin-dependent patient” or “insulin-dependent diabetes” encompasses all forms of diabetics/diabetes who/that require insulin administration to adequately maintain normal glucose levels.

In broad terms, the term “diabetes” refers to the situation where the body either fails to properly respond to its own insulin, does not make enough insulin, or both. The primary result of impaired insulin production is the accumulation of glucose in the blood, and a C-peptide deficiency leading to various short- and long-term complications. Three principal forms of diabetes exist:

Type 1: Results from the body's failure to produce insulin and C-peptide. It is estimated that 5-10% of Americans who are diagnosed with diabetes have type 1 diabetes. Presently almost all persons with type 1 diabetes must take insulin injections. The term “type 1 diabetes” has replaced several former terms, including childhood-onset diabetes, juvenile diabetes, and insulin-dependent diabetes mellitus (IDDM). For patients with type 1 diabetes, basal levels of C-peptide are typically less than about 0.20 nM (Ludvigsson et al.: New Engl. J. Med. 359: 1909-1920, (2008)).

Type 2: Results from tissue insulin resistance, a condition in which cells fail to respond properly to insulin, sometimes combined with relative insulin deficiency. The term “type 2 diabetes” has replaced several former terms, including adult-onset diabetes, obesity-related diabetes, and non-insulin-dependent diabetes mellitus (NIDDM). For type 2 patients in the basal state, C-peptide levels of about 0.8 nM (range 0.64 to 1.56 nM), and glucose stimulated levels of about 5.7 nM (range 3.7 to 7.7 nM) have been reported. (Retnakaran R et al.: Diabetes Obes. Metab. (2009) DOI 10.11 111/j.1463-1326.2009.01129.x; Zander et al.: Lancet 359: 824-830, (2002)).

In addition to type 1 and type 2 diabetics, there is increasing recognition of a subclass of diabetes referred to as latent autoimmune diabetes in the adult (LADA) or Late-onset Autoimmune Diabetes of Adulthood, or “Slow Onset Type 1” diabetes, and sometimes also “Type 1.5” or “Type one-and-a-half” diabetes. In this disorder diabetes onset generally occurs in ages 35 and older, and antibodies against components of the insulin-producing cells are always present, demonstrating that autoimmune activity is an important feature of LADA. It is primarily antibodies against glutamic acid decarboxylase (GAD) that are found. Some LADA patients show a phenotype similar to that of type 2 patients with increased body mass index (BMI) or obesity, insulin resistance, and abnormal blood lipids. Genetic features of LADA are similar to those for both type 1 and type 2 diabetes. During the first 6-12 months after debut the patients may not require insulin administration and they are able to maintain relative normoglycemia via dietary modification and/or oral anti-diabetic medication. However, eventually all patients become insulin dependent, probably as a consequence of progressive autoimmune activity leading to gradual destruction of the pancreatic islet β-cells. At this stage the LADA patients show low or absent levels of endogenous insulin and C-peptide, and they are prone to develop long-term complications of diabetes involving the peripheral nerves, the kidneys, or the eyes similar to type 1 diabetes patients and thus become candidates for C-peptide therapy (Palmer et al.: Diabetes 54(suppl 2): S62-67, (2005); Desai et al.: Diabetic Medicine 25(suppl 2): 30-34, (2008); Fourlanos et al.: Diabetologia 48: 2206-2212, (2005)).

Gestational diabetes: Pregnant women who have never had diabetes before but who have high blood sugar (glucose) levels during pregnancy are said to have gestational diabetes. Gestational diabetes affects about 4% of all pregnant women. It may precede development of type 2 (or rarely type 1).

Several other forms of diabetes mellitus are categorized separately from these. Examples include congenital diabetes due to genetic defects of insulin secretion, cystic fibrosis-related diabetes, steroid diabetes induced by high doses of glucocorticoids, and several forms of monogenic diabetes.

Acute complications of diabetes include hypoglycemia, diabetic ketoacidosis, or nonketotic hyperosmolar coma that may occur if the disease is not adequately controlled. Serious long-term complications can also occur, and are discussed in more detail below.

Long-Term Complications of Diabetes

In any of these methods, the terms “long-term complication of type 1 diabetes”, or “long term complications of diabetes” refers to the long-term complications of impaired glycemic control, and C-peptide deficiency associated with insulin-dependent diabetes. Typically long-term complications of type 1 diabetes are associated with type 1 diabetics. However the term can also refer to long-term complications of diabetes that arise in type 1.5 and type 2 diabetic patients who develop a C-peptide deficiency as a consequence of losing pancreatic islet p-cells and therefore also become insulin dependent. In broad terms, many such complications arise from the primary damage of blood vessels (angiopathy), resulting in subsequent problems that can be grouped under “microvascular disease” (due to damage to small blood vessels) and “macrovascular disease” (due to damage to the arteries).

Specific diseases and disorders included within the term long-term complications of diabetes include, without limitation; retinopathy including early stage retinopathy with microaneurysms, proliferative retinopathy, and macular edema; peripheral neuropathy including sensorimotor polyneuropathy, painful sensory neuropathy, acute motor neuropathy, cranial focal and multifocal polyneuropathies, thoracolumbar radiculoneuropathies, proximal diabetic neuropathies, and focal limb neuropathies including entrapment and compression neuropathies; autonomic neuropathy involving the cardiovascular system, the gastrointestinal tract, the respiratory system, the urigenital system, sudomotor function and papillary function; and nephropathy including disorders with microalbuminuria, overt proteinuria, and end-stage renal disease.

Impaired microcirculatory perfusion appears to be crucial to the pathogenesis of both neuropathy and retinopathy in diabetics. This in turn reflects a hyperglycemia-mediated perturbation of vascular endothelial function that results in: over-activation of protein kinase C, reduced availability of nitric oxide (NO), increased production of superoxide and endothelin-1 (ET-1), impaired insulin function, diminished synthesis of prostacyclin/PGE1, and increased activation and endothelial adherence of leukocytes. This is ultimately a catastrophic group of clinical events.

Accordingly in any of these methods, the term “patient” refers to an individual who has one of more of the symptoms of the long term complications of diabetes.

Diabetic retinopathy is an ocular manifestation of the systemic damage to small blood vessels leading to microangiopathy. In retinopathy, growth of friable and poor-quality new blood vessels in the retina as well as macular edema (swelling of the macula) can lead to severe vision loss or blindness. As new blood vessels form at the back of the eye as a part of proliferative diabetic retinopathy (PDR), they can bleed (hemorrhage) and blur vision. It affects up to 80% of all patients who have had diabetes for 10 years or more.

The symptoms of diabetic retinopathy are often slow to develop and subtle and include blurred version and progressive loss of sight. Macular edema, which may cause vision loss more rapidly, may not have any warning signs for some time. In general, however, a person with macular edema is likely to have blurred vision, making it hard to do things like read or drive. In some cases, the vision will get better or worse during the day.

Accordingly in any of these methods, the term “patient” refers to an individual who has one of more of the symptoms of diabetic retinopathy.

Diabetic neuropathies are neuropathic disorders that are associated with diabetic microvascular injury involving small blood vessels that supply nerves (vasa nervorum). Relatively common conditions which may be associated with diabetic neuropathy include third nerve palsy; mononeuropathy; mononeuropathy multiplex; diabetic amyotrophy; a painful polyneuropathy; autonomic neuropathy; and thoracoabdominal neuropathy.

Diabetic neuropathy affects all peripheral nerves: pain fibers, motor neurons, autonomic nerves. It therefore necessarily can affect all organs and systems since all are innervated. There are several distinct syndromes based on the organ systems and members affected, but these are by no means exclusive. A patient can have sensorimotor and autonomic neuropathy or any other combination. Symptoms vary depending on the nerve(s) affected and may include symptoms other than those listed. Symptoms usually develop gradually over years.

Symptoms of diabetic neuropathy may include: numbness and tingling of extremities, dysesthesia (decreased or loss of sensation to a body part), diarrhea, erectile dysfunction, urinary incontinence (loss of bladder control), impotence, facial, mouth and eyelid drooping, vision changes, dizziness, muscle weakness, difficulty swallowing, speech impairment, fasciculation (muscle contractions), anorgasmia, and burning or electric pain.

Additionally, different nerves are affected in different ways by neuropathy. Sensorimotor polyneuropathy, in which longer nerve fibers are affected to a greater degree than shorter ones, because nerve conduction velocity is slowed in proportion to a nerve's length. In this syndrome, decreased sensation and loss of reflexes occurs first in the toes on each foot, then extends upward. It is usually described as glove-stocking distribution of numbness, sensory loss, dysesthesia, and nighttime pain. The pain can feel like burning, pricking sensation, achy, or dull. Pins and needles sensation is common. Loss of proprioception, the sense of where a limb is in space, is affected early. These patients cannot feel when they are stepping on a foreign body, like a splinter, or when they are developing a callous from an ill-fitting shoe. Consequently, they are at risk for developing ulcers and infections on the feet and legs, which can lead to amputation. Similarly, these patients can get multiple fractures of the knee, ankle, or foot, and develop a Charcot joint. Loss of motor function results in dorsiflexion, contractures of the toes, loss of the interosseous muscle function, and leads to contraction of the digits, so called hammer toes. These contractures occur not only in the foot, but also in the hand where the loss of the musculature makes the hand appear gaunt and skeletal. The loss of muscular function is progressive.

Autonomic neuropathy impacts the autonomic nervous system serving the heart, gastrointestinal system, and genitourinary system. The most commonly recognized autonomic dysfunction in diabetics is orthostatic hypotension, or fainting when standing up. In the case of diabetic autonomic neuropathy, it is due to the failure of the heart and arteries to appropriately adjust heart rate and vascular tone to keep blood continually and fully flowing to the brain. This symptom is usually accompanied by a loss of the usual change in heart rate seen with normal breathing. These two findings suggest autonomic neuropathy.

Gastrointestinal system symptoms include delayed gastric emptying, gastroparesis, nausea, bloating, and diarrhea. Because many diabetics take oral medication for their diabetes, absorption of these medicines is greatly affected by the delayed gastric emptying. This can lead to hypoglycemia when an oral diabetic agent is taken before a meal and does not get absorbed until hours, or sometimes days later, when there is normal or low blood sugar already. Sluggish movement of the small intestine can cause bacterial overgrowth, made worse by the presence of hyperglycemia. This leads to bloating, gas, and diarrhea.

Genitourinary system symptoms include urinary frequency, urgency, incontinence, and retention. Urinary retention can lead to bladder diverticula, stones, reflux nephropathy, and frequent urinary tract infections. Accordingly in any of these methods, the term “patient” refers to an individual who has one of more of the symptoms of autonomic neuropathy.

Accordingly in any of these methods, the term “patient” refers to an individual who has one of more of the symptoms of diabetic neuropathy. In another aspect of any of these methods, the patient has “established peripheral neuropathy” which is characterized by reduced sensory nerve conduction velocity (SCV) in the sural nerves (less than −1.5 SD from a body height-corrected reference value for a matched normal individual.

Diabetic nephropathy is a progressive kidney disease caused by angiopathy of capillaries in the kidney glomeruli. It is characterized by nephrotic syndrome and diffuse glomerulosclerosis. It is due to long-standing diabetes mellitus, and is a prime cause for dialysis in many Western countries.

The symptoms of diabetic nephropathy can be seen in patients with chronic diabetes (15 years or more after onset). The disease is progressive and is more frequent in men. Diabetic nephropathy is the most common cause of chronic kidney failure and end-stage kidney disease in the United States. People with both type 1 and type 2 diabetes are at risk. The risk is higher if blood-glucose levels are poorly controlled. Further, once nephropathy develops, the greatest rate of progression is seen in patients with poor control of their blood pressure. Also people with high cholesterol level in their blood have much more risk than others.

The earliest detectable change in the course of diabetic nephropathy is an abnormality of the glomerular filtration barrier. At this stage, the kidney may start allowing more serum albumin than normal in the urine (albuminuria), and this can be detected by sensitive medical tests for albumin. This stage is called “microalbuminuria”. As diabetic nephropathy progresses, increasing numbers of glomeruli are destroyed by nodular glomerulosclerosis. Now the amounts of albumin being excreted in the urine increases, and may be detected by ordinary urinalysis techniques. At this stage, a kidney biopsy clearly shows diabetic nephropathy.

Kidney failure provoked by glomerulosclerosis leads to fluid filtration deficits and other disorders of kidney function. There is an increase in blood pressure (hypertension) and fluid retention in the body plus a reduced plasma oncotic pressure causes edema. Other complications may be arteriosclerosis of the renal artery and proteinuria.

Throughout its early course, diabetic nephropathy has no symptoms. They develop in late stages and may be a result of excretion of high amounts of protein in the urine or due to renal failure. Symptoms include, edema: swelling, usually around the eyes in the mornings; later, general body swelling may result, such as swelling of the legs, foamy appearance or excessive frothing of the urine (caused by the proteinuria), unintentional weight gain (from fluid accumulation), anorexia (poor appetite), nausea and vomiting, malaise (general ill feeling), fatigue, headache, frequent hiccups, and generalized itching.

Accordingly in any of these methods, the term “patient” refers to an individual who has one of more of the symptoms of diabetic nephropathy.

Diabetic cardiomyopathy (DCM), damage to the heart, leading to diastolic dysfunction and eventually heart failure. Aside from large vessel disease and accelerated atherosclerosis, which is very common in diabetes, DCM is a clinical condition diagnosed when ventricular dysfunction develops in patients with diabetes in the absence of coronary atherosclerosis and hypertension. DCM may be characterized functionally by ventricular dilation, myocyte hypertrophy, prominent interstitial fibrosis, and decreased or preserved systolic function in the presence of a diastolic dysfunction.

One particularity of DCM is the long latent phase, during which the disease progresses but is completely asymptomatic. In most cases, DCM is detected with concomitant hypertension or coronary artery disease. One of the earliest signs is mild left ventricular diastolic dysfunction with little effect on ventricular filling. Also, the diabetic patient may show subtle signs of DCM related to decreased left ventricular compliance or left ventricular hypertrophy or a combination of both. A prominent “a” wave can also be noted in the jugular venous pulse, and the cardiac apical impulse may be overactive or sustained throughout systole. After the development of systolic dysfunction, left ventricular dilation and symptomatic heart failure, the jugular venous pressure may become elevated and the apical impulse would be displaced downward and to the left. Systolic mitral murmur is not uncommon in these cases. These changes are accompanied by a variety of electrocardiographic changes that may be associated with DCM in 60% of patients without structural heart disease, although usually not in the early asymptomatic phase. Later in the progression, a prolonged QT interval may be indicative of fibrosis. Given that DCM's definition excludes concomitant atherosclerosis or hypertension, there are no changes in perfusion or in atrial natriuretic peptide levels up until the very late stages of the disease, when the hypertrophy and fibrosis become very pronounced.

Macrovascular diseases of diabetes include coronary artery disease, leading to angina or myocardial infarction (“heart attack”), stroke (mainly the ischemic type), peripheral vascular disease, which contributes to intermittent claudication (exertion-related leg and foot pain), as well as diabetic foot and diabetic myonecrosis (“muscle wasting”).

Therapeutic Forms of C-Peptide

The terms “C-peptide” or “proinsulin C-peptide” as used herein includes all naturally-occurring and synthetic forms of C-peptide that retain C-peptide activity. Such C-peptides include the human peptide, as well as peptides derived from other animal species and genera, preferably mammals. Preferably, “C-peptide” refers to human C-peptide having the amino acid sequence EAEDLQVGQVELGGGPGAGSLQPLALEGSLQ (SEQ. ID. NO. 1 in Table D1).

C-peptides from a number of different species have been sequenced, and are known in the art to be at least partially functionally interchangeable. It would thus be a routine matter to select a variant being a C-peptide from a species or genus other than human. Several such variants of C-peptide (i.e., representative C-peptides from other species) are shown in Table D1 (see Seq ID Nos. 1-29).

TABLE D1
C-Peptide Variants

| | | |
|---|---|---|
| human M- | Human EAEDLQVGQVELGGGPGAGSLQPLALEGSLQ | gb\|AAA72531.1\| |
| proinsulin | (SEQ. ID. NO. 1) | dbj\|BAH59081.1\| |
| Pan | (SEQ. ID. NO. 1) EAEDLQVGQVELGGGPGAGSLQPLALEGSLQ | NP_001008996.1\| |
| troglodytes | Alignment EAEDLQVGQVELGGGPGAGSLQPLALEGSLQ | emb\|CAA43403.1\| |
| | (SEQ. ID. NO. 2) EAEDLQVGQVELGGGPGAGSLQPLALEGSLQ | GENE ID: 449570 |
| | Identities = 31/31 (100%), | INS |
| | Positives = 31/31 (100%), | |
| | Gaps = 0/31 (0%) | |
| Gorilla | (SEQ. ID. NO. 1) EAEDLQVGQVELGGGPGAGSLQPLALEGSLQ | gb\|AAN06935.1\| |
| gorilla | Alignment EAEDLQVGQVELGGGPGAGSLQPLALEGSLQ | |
| | (SEQ. ID. NO. 3) EAEDLQVGQVELGGGPGAGSLQPLALEGSLQ | |
| | Identities = 31/31 (100%), Positives = 31/31 | |
| | (100%), Gaps = 0/31 (0%) | |
| Pongo | (SEQ. ID. NO. 1) EAEDLQVGQVELGGGPGAGSLQPLALEGSLQ | gb\|AAN06937.1\| |
| pygmaeus | EAEDLQVGQVELGGGPGAGSLQPLALEGSLQ | |
| (Bornean | (SEQ. ID. NO. 4) EAEDLQVGQVELGGGPGAGSLQPLALEGSLQ | |
| orangutan) | Identities = 31/31 (100%), Positives = 31/31 | |
| | (100%), Gaps = 0/31 (0%) | |
| Chlorocebus | (SEQ. ID. NO. 1) EAEDLQVGQVELGGGPGAGSLQPLALEGSLQ | emb\|CAA43405.1\| |
| aethiops | EAED QVGQVELGGGPGAGSLQPLALEGSLQ | |
| (Monkey) | (SEQ. ID. NO. 5) EAEDPQVGQVELGGGPGAGSLQPLALEGSLQ | |
| | Identities = 30/31 (96%), Positives = 30/31 | |
| | (96%), Gaps = 0/31 (0%) | |
| Canis lupus | (SEQ. ID. NO. 1) EAEDLQVGQVELGGGPGAGSLQPLALEGSLQ | ref\|NP_001123565.1\| |
| familiaris | E EDLQV VEL G PG G LQPLALEG+LQ | sp\|P01321.1\| |
| (Dog) | (SEQ. ID. NO. 6) EVEDLQVRDVELAGAPGEGGLQPLALEGALQ | INS_CANFAem |
| | Identities = 23/31(74%), Positives = 24/31 | b\|CAA23475.1\| |
| | (77%), Gaps = 0/31 (0%) | GENE ID: 483665 |
| | | INS |
| Oryctolagus | (SEQ. ID. NO. 1) EAEDLQVGQVELGGGPGAGSLQPLALEGSLQ | gb\|ACK44319.1\| |
| cuniculus | E E+LQVGQ ELGGGP AG LQP ALE +LQ | |
| (Rabbit) | (SEQ. ID. NO. 7) EVEELQVGQAELGGGPDAGGLQPSALELALQ | |
| | Identities = 23/31(74%), Positives = 25/31 | |
| | (80%), Gaps = 0/31 (0%) | |
| Rattus | (SEQ.ID. NO. 1) EAEDLQVGQVELGGGPGAGSLQPLALEGSLQ | ref\|NP_062003.1\| |
| norvegicus | E ED QV Q+ELGGGPGAG LQ LALE + Q | sp\|P01323.1\|IN |
| | (SEQ. ID. NO.8) EVEDPQVAQLELGGGPGAGDLQTLALEVARQ | S2_RAT |
| | Identities = 22/31(70%), Positives = 24/31 | emb\|CAA24560.1\| |
| | (77%), Gaps = 0/31 (0%) | GENE ID: 24506 |
| | | Ins2 |
| Apodemus | (SEQ. ID. NO. 1) EAEDLQVGQVELGGGPGAGSLQPLALEGSLQ | gb\|ABB89748.1\| |
| semotus | E ED QV Q+ELGGGPGAG LQ LALE + Q | |
| (Taiwan | (SEQ. ID. NO. 9) EVEDPQVAQLELGGGPGAGDLQTLALEVARQ | |
| field | Identities = 22/31 (70%), Positives = 24/31 | |
| mouse) | (77%), Gaps = 0/31 (0%) | |
| Geodia | (SEQ. ID. NO. 1) EAEDLQVGQVELGGGPGAGSLQPLALEGSLQ | pir\|\|S09278 |
| cydonium | E ED QVGQVELG GPGAGS Q LALE + Q | |
| sponge | (SEQ. ID. NO. 10)EVEDPQVGQVELGAGPGAGSEQTLALEVARQ | |
| | Identities = 23/31 (74%), Positives = 24/31 | |
| | (77%), Gaps = 0/31 (0%) | |
| Mus | (SEQ. ID. NO. 1) EAEDLQVGQVELGGGPGAGSLQPLALE | ref\|NP_032413.1\| |
| musculus | E ED QV Q+ELGGGPGAG LQ LALE | sp\|P01326.1\|IN |
| | (SEQ. ID. NO. 11) EVEDPQVAQLELGGGPGAGDLQTLALE | S2_MOUSEemb\|CA |
| | Identities = 21/27 (77%), Positives = 22/27 | A28433.1\| |
| | (81%), Gaps = 0/27 (0%) | GENE ID: 16334 |
| | | Ins2 |
| Mus caroli | (SEQ.ID. NO. 1) EAEDLQVGQVELGGGPGAGSLQPLALE | gb\|ABB89749.1\| |
| (Ryukyu | E ED QV Q+ELGGGPGAG LQ LALE | |
| mouse) | (SEQ.ID. NO. 12) EVEDPQVAQLELGGGPGAGDLQTLALE | |
| | Identities = 21/27 (77%), Positives = 22/27 | |
| | (81%), Gaps = 0/27 (0%) | |
| Rattus | (SEQ. ID. NO. 1) EAEDLQVGQVELGGGPGAGSLQPLALEGSLQ | prf\|\|720460B |
| norvegicus | E ED QV Q+ELGGGPGAG LQ LALE + Q | |
| | (SEQ. ID. NO. 13) EVEDPQVPQLELGGGPGAGDLQTLALEVARQ | |
| | Identities = 22/31 (70%), Positives = 24/31 | |
| | (77%), Gaps = 0/31 (0%) | |
| Rattus | (SEQ. ID. NO. 1) EAEDLQVGQVELGGGPGAGSLQPLALEGSLQ | gb\|ABB89747.1\| |
| losea | E ED QV Q ELGGGPGAG LQ LALE + Q | |
| | (SEQ. ID. NO. 14) EVEDPQVAQQELGGGPGAGDLQTLALEVARQ | |
| | Identities = 22/31 (70%), Positives = 23/31 | |
| | (74%), Gaps = 0/31 (0%) | |
| Niviventer | (SEQ. ID. NO. 1) EAEDLQVGQVELGGGPGAGSLQPLALEGSLQ | gb\|ABB89750.1\| |
| coxingi | E ED QV Q+ELGGGPG G LQ LALE + Q | |
| (Coxing's | (SEQ. ID. NO. 15) EVEDPQVPQLELGGGPGTGDLQTLALEVARQ | |
| white- | Identities = 21/31 (67%), Positives = 23/31 | |
| bellied | (74%), Gaps = 0/31 (0%) | |
| rat) | | |
| Microtus | (SEQ. ID. NO. 1) AEDLQVGQVELGGGPGAGSLQPLALE | gb\|ABB89752.1\| |
| kikuchii | ED QV Q+ELGGGPGAG LQ LALE | |
| (Taiwan | (SEQ. ID. NO. 16) VEDPQVAQLELGGGPGAGDLQTLALE | |
| vole) | Identities = 20/26 (76%), Positives = 21/26 | |
| | (80%), Gaps = 0/26 (0%) | |
| Rattus | (SEQ. ID. NO. 1) EAEDLQVGQVELGGGPGAGSLQPLALEGSLQ | ref\|NP_062002.1\| |
| norvegicus | E ED QV Q+ELGGGP AG LQ LALE + Q | gb\|AAA41439.1\| |
| insulin 1 | (SEQ. ID. NO. 17) EVEDPQVPQLELGGGPEAGDLQTLALEVARQ | gb\|AAA41442.1\| |
| precursor | Identities = 21/31 (67%), Positives = 23/31 | emb\|CAA24559.1\| |
| | (74%), Gaps = 0/31 (0%) | gb\|EDL94407.1\| |
| | | GENE ID: 24505 |
| | | Ins1 |
| Felis catus | (SEQ. ID. NO. 1) EAEDLQVGQVELGGGPGAGSLQPLALEGSLQ | ref\|NP_001009272.1\| |
| (Domestic | EAEDLQ ELG PGAG LQP ALE LQ | sp\|P06306.2\|IN |
| cat) | (SEQ. ID. NO. 18) EAEDLQGKDAELGEAPGAGGLQPSALEAPLQ | S_FELCA |
| | Identities = 21/31 (67%), Positives = 21/31 | dbj\|BAB84110.1\| |
| | (67%), Gaps = 0/31 (0%) | GENE ID:493804 |
| | | INS |
| Golden | (SEQ. ID. NO. 1) AEDLQVGQVELGGGPGAGSLQPLALE | sp\|P01313.2\|IN |
| hamster | ED QV Q+ELGGGPGA LQ LALE | S_CRILO |
| | (SEQ. ID. NO. 19) VEDPQVAQLELGGGPGADDLQTLALE | pir\|\|I48166 |
| | Identities = 19/26 (73%), Positives = 20/26 | gb\|AAA37089.1\| |
| | (76%), Gaps = 0/26 (0%) | |
| Niviventer | (SEQ. ID. NO. 1) EAEDLQVGQVELGGGPGAGSLQPLALEGSLQ | gb\|ABB89746.1\| |
| coxingi | E ED QV Q+ELG GP AG LQ LALE + Q | |
| (Coxing's | (SEQ. ID. NO. 20) EVEDPQVAQLELGEGPEAGDLQTLALEVARQ | |
| white- | Identities = 20/31 (64%), Positives = 22/31 | |
| bellied | (70%), Gaps = 0/31 (0%) | |
| rat) | | |
| Apodemus | (SEQ. ID. NO. 1) EAEDLQVGQVELGGGPGAGSLQPLALEGSLQ | gb\|ABB89744.1\| |
| semotus | E ED QV Q+ELGG PG G L+ LALE + Q | |
| (Taiwan | (SEQ. ID. NO. 21) EVEDPQVEQLELGGAPGTGDLETLALEVARQ | |
| field | Identities = 19/31 (61%), Positives = 22/31 | |
| mouse) | (70%), Gaps = 0/31 (0%) | |
| Rattus | (SEQ. ID. NO. 1) EAEDLQVGQVELGGGPGAGSLQPLALEGSLQ | gb\|ABB89743.1\| |
| losea | E ED QV Q+ELGG P AG LQ LALE + Q | |
| | (SEQ. ID. NO. 22) EVEDPQVPQLELGGSPEAGDLQTLALEVARQ | |
| | Identities = 20/31 (64%), Positives = 22/31 | |
| | (70%), Gaps = 0/31 (0%) | |
| Meriones | (SEQ. ID. NO. 1) AEDLQVGQVELGGGPGAGSLQPLALEGSLQ | gb\|ABB89751.1\| |
| unguiculatus | ED Q+ Q+ELGG PGAG LQ LALE + Q | |
| (Mongolian | (SEQ. ID. NO. 23) VEDPQMPQLELGGSPGAGDLQALALEVARQ | |
| gerbil) | Identities = 19/30 (63%), Positives = 22/30 | |
| | (73%), Gaps = 0/30 (0%) | |
| Psammomys | (SEQ. ID. NO. 1) AEDLQVGQVELGGGPGAGSLQPLALEGSLQ | sp\|Q62587.1\|IN |
| obesus | +D Q+ Q+ELGG PGAG L+ LALE + Q | S_PSAOB |
| (Fat sand | (SEQ. ID. NO. 24) VDDPQMPQLELGGSPGAGDLRALALEVARQ | emb\|CAA66897.1\| |
| rat) | Identities = 17/30 (56%), Positives = 22/30 | |
| | (73%), Gaps = 0/30 (0%) | |
| Sus scrofa | (SEQ. ID. NO. 1) EAEDLQVGQVELGGGPGAGSLQPLALEG | ref\|NP_001103242.1\| |
| (Pig) | EAE+ Q G VELGG G G LQ LALEG | |
| | (SEQ. ID. NO. 25) EAENPQAGAVELGG--GLGGLQALALEG | |
| | Identities = 19/28 (67%), Positives = 20/28 | |
| | (71%), Gaps = 2/28 (7%) | |
| Rhinolophus | (SEQ. ID. NO. 26) | gb\|ACC68945.1\| |
| ferrumequinum | EVEDPQAGQVELGGGPGTGGLQSLALEGPPQ | |
| Equus | (SEQ. ID. NO. 27) | GENE ID: |
| przewalskii | EAEDPQVGEVELGGGPGLGGLQPLALAGPQQ | 100060077 |
| (Horse) | | LOC100060077 |
| | | gb\|AAB25818.1\| |
| Bos Taurus | (SEQ. ID. NO. 28) EVEGPQVGALELAGGPGAGGLEGPPQ | gb\|AAI42035.1\| |
| (Bovine) | | |
| Otolemur | (SEQ. ID. NO. 29) | gb\|ACH53103.1\| |
| garnettii | DTEDPQVGQVGLGGSPITGDLQSLALDVPPQ | |
| (Small- | | |
| eared | | |
| galago) | | |

Thus all such homologues, orthologs, and naturally-occurring isoforms of C-peptide from human as well as other species (Seq ID Nos. 1-29) are included in any of the methods of the invention, as long as they retain detectable C-peptide activity.

The C-peptides may be in their native form, i.e., as different variants as they appear in nature in different species which may be viewed as functionally equivalent variants of human C-peptide, or they may be functionally equivalent natural derivatives thereof, which may differ in their amino acid sequence, e.g., by truncation (e.g., from the N- or C-terminus or both) or other amino acid deletions, additions, insertions, substitutions, or post-translational modifications. Naturally-occurring chemical derivatives, including post-translational modifications and degradation products of C-peptide, are also specifically included in any of the methods of the invention including, e.g., pyroglutamyl, iso-aspartyl, proteolytic, phosphorylated, glycosylated, oxidatized, isomerized, and deaminated variants of C-peptide.

It is known in the art to synthetically modify the sequences of proteins or peptides, while retaining their useful activity, and this may be achieved using techniques which are standard in the art and widely described in the literature, e.g., random or site-directed mutagenesis, cleavage, and ligation of nucleic acids, or via the chemical synthesis or modification of amino acids or polypeptide chains. Similarly it is within the skill in the art to address and/or mitigate immunogenicity concerns if they arise using C-peptide variants, e.g., by the use of automated computer recognition programs to identify potential T cell epitopes, and directed evolution approaches to identify less immunogenic forms.

Any such modifications, or combinations thereof, may be made and used in any of the methods of the invention, as long as activity is retained. The C-terminal end of the molecule is known to be important for activity. Preferably, therefore, the C-terminal end of the C-peptide should be preserved in any such C-peptide variants or derivatives, more preferably the C-terminal pentapeptide of C-peptide (EGSLQ) (SEQ. ID. NO. 31) should be preserved or sufficient (see Henriksson M et al.: Cell Mol. Life. Sci. 62: 1772-1778, (2005)). As mentioned above, modification of an amino acid sequence may be by amino acid substitution, e.g., an amino acid may be replaced by another that preserves the physicochemical character of the peptide (e.g., A may be replaced by -G or vice versa, V by A or L; E by D or vice versa; and Q by N). Generally, the substituting amino acid has similar properties, e.g., hydrophobicity, hydrophilicity, electronegativity, bulky side chains, etc., to the amino acid being replaced.

Modifications to the mid-part of the C-peptide sequence (e.g., to residues 13 to 25 of human C-peptide) allow the production of functional derivatives or variants of C-peptide. Thus, C-peptides which may be used in any of the methods of the invention may have amino acid sequences which are substantially homologous, or substantially similar to the native C-peptide amino acid sequences, e.g., to the human C-peptide sequence of SEQ. ID. NO. 1 or any of the other native C-peptide sequences shown in Table D1. Alternatively, the C-peptide may have an amino acid sequence having at least 30% preferably at least 40, 50, 60, 70, 75, 80, 85, 90, 95, 98, or 99% identity with the amino acid sequence of any one of SEQ. ID. NOs. 1-29 as shown in Table D1, preferably with the native human sequence of SEQ. ID. NO. 1. In a preferred embodiment, the C-peptide for use in any of the methods of the present invention is at least 80% identical to a sequence selected from Table D1. In another aspect, the C-peptide for use in any of the methods of the invention is at least 80% identical to human C-peptide (SEQ. ID. NO. 1). Although any amino acid of C-peptide may be altered as described above, it is preferred that one or more of the glutamic acid residues at positions 3, 11, and 27 of human C-peptide (SEQ. ID. NO. 1) or corresponding or equivalent positions in C-peptide of other species, are conserved. Preferably, all of the glutamic acid residues at positions 3, 11, and 27 (or corresponding Glu residues) of SEQ. ID. NO. 1 are conserved. Alternatively, it is preferred that Glu27 of human C-peptide (or a corresponding Glu residue of a non-human C-peptide) is conserved. An exemplary functional equivalent form of C-peptide which may be used in any of the methods of the invention includes the amino acid sequences:

(SEQ. ID. NO. 30)
EXEXXQXXXXELXXXXXXXXXXXXALBXXXQ.
(SEQ. ID. NO. 33)
GXEXXQXXXXELXXXXXXXXXXXXALBXXXQ.

As used herein, “X” is any amino acid. The N-terminal residue may be either Glu or Gly (SEQ. ID NO. 30 or SEQ. ID NO. 33 respectively). Functionally equivalent derivatives or variants of native C-peptide sequences may readily be prepared according to techniques well-known in the art, and include peptide sequences having a functional, e.g., a biological activity of a native C-peptide.

Fragments of native or synthetic C-peptide sequences may also have the desirable functional properties of the peptide from which they were derived and may be used in any of the methods of the invention. The term “fragment” as used herein thus includes fragments of a C-peptide provided that the fragment retains the biological or therapeutically beneficial activity of the whole molecule. The fragment may also include a C-terminal fragment of C-peptide. Preferred fragments comprise residues 15-31 of native C-peptide, more especially residues 20-31. Peptides comprising the pentapeptide EGSLQ (SEQ. ID. NO. 31) (residues 27-31 of native human C-peptide) are also preferred. The fragment may thus vary in size from, e.g., 4 to 30 amino acids or 5 to 20 residues. Suitable fragments are disclosed in WO 98/13384 the contents of which are incorporated herein by reference.

The fragment may also include an N-terminal fragment of C-peptide, typically having the sequence EAEDLQVGQVEL (SEQ. ID. NO. 32), or a fragment thereof which comprises 2 acidic amino acid residues, capable of adopting a conformation where said two acidic amino acid residues are spatially separated by a distance of 9-14 A between the alpha-carbons thereof. Also included are fragments having N- and/or C-terminal extensions or flanking sequences. The length of such extended peptides may vary, but typically are not more than 50, 30, 25, or 20 amino acids in length. Representative suitable fragments are described in U.S. Pat. No. 6,610,649, which is hereby incorporated by reference in its entirety.

In such a case it will be appreciated that the extension or flanking sequence will be a sequence of amino acids which is not native to a naturally-occurring or native C-peptide, and in particular a C-peptide from which the fragment is derived. Such a N- and/or C-terminal extension or flanking sequence may comprise, e.g., from 1 to 10, e.g., 1 to 6, 1 to 5, 1 to 4, or 1 to 3 amino acids.

The term “derivative” as used herein thus refers to C-peptide sequences or fragments thereof, which have modifications as compared to the native sequence. Such modifications may be one or more amino acid deletions, additions, insertions, and/or substitutions. These may be contiguous or non-contiguous. Representative variants may include those having 1 to 6, or more preferably 1 to 4, 1 to 3, or 1 or 2 amino acid substitutions, insertions, and/or deletions as compared to any of SEQ. ID. NOs. 1-33. The substituted amino acid may be any amino acid, particularly one of the well-known 20 conventional amino acids (Ala (A); Cys (C); Asp (D); Glu (E); Phe (F); Gly (G); His (H); Ile (I); Lys (K); Leu (L); Met (M); Asn (N); Pro (P); Gin (Q); Arg (R); Ser (S); Thr (T); Val (V); Trp (W); and Tyr (Y)). Any such variant or derivative of C-peptide may be used in any of the methods of the invention.

Fusion proteins of C-peptide to other proteins are also included, and these fusion proteins may enhance C-peptide's biological activity, targeting, biological life, or pharmacokinetic properties. Examples of fusion proteins that improve pharmacokinetic properties include without limitation, fusions to human albumin (Osborn et al.: Eur. J. Pharmacol. 456(1-3): 149-158, (2002)), antibody fc domains, poly Glu or poly Asp sequences, and transferrin. Additionally, fusion with conformationally disordered polypeptide sequences composed of the amino acids Pro, Ala, and Ser ('PASylation') or hydroxyethyl starch (HESylation®) provides a simple way to increase the hydrodynamic volume of the C-peptide. This additional extension adopts a bulky random structure, which significantly increases the size of the resulting fusion protein. By this means the typically rapid clearance of the C-peptide via kidney filtration is retarded by several orders of magnitude.

An additional fusion protein approach contemplated for use within the present invention includes the fusion of C-peptide to a multimerization domain. Representative multimerization domains include without limitation coiled-coil dimerization domains such as leucine zipper domains which are found in certain DNA-binding polypeptides, the dimerization domain of an immunoglobulin Fab constant domain, such as an immunoglobulin heavy chain CH1 constant region or an immunoglobulin light chain constant region. In a preferred embodiment, the multimerisation domain is derived from tetranectin, and more specifically comprises the tetranectin trimerising structural element, which is described in detail in WO 98/56906.

It will be appreciated that a flexible molecular linker (or spacer) optionally may be interposed between, and covalently join, the C-peptide and any of the fusion proteins disclosed herein. Any such fusion protein many be used in any of the methods of the present invention.

Chemical modifications of the native C-peptide structure, which retain or stabilize C-peptide activity or biological half-life may also be used with any of the methods described herein. Such chemical modification strategies include, without limitation, pegylation, glycosylation, and acylation (Clark et al.: J. Biol. Chem. 271(36): 21969-21977, (1996); Roberts et al.: Adv. Drug. Deliv. Rev. 54(4): 459-476, (2002); Felix et al.: Int. J. Pept. Protein. Res. 46(3-4): 253-264, (1995); Garber A J: Diabetes Obes. Metab. 7(6): 666-74 (2005)). C- and N-terminal protecting groups and peptomimetic units may also be included.

A wide variety of PEG derivatives are both available and suitable for use in the preparation of PEG-conjugates, and for use in any of the methods disclosed herein. For example, NOF Corp.'s SUNBRIGHT® Series provides numerous PEG derivatives, including methoxypolyethylene glycols and activated PEG derivatives such as methoxy-PEG amines, maleimides, and carboxylic acids, for coupling by various methods to drugs, enzymes, phospholipids, and other biomaterials and Nektar Therapeutics' Advanced PEGylation also offers diverse PEG-coupling technologies to improve the safety and efficacy of therapeutics. Exemplary PEGylated C-peptides are disclosed for example in commonly owned U.S. provisional application No. 61/345,293 filed May 17, 2010 entitled “PEGYLATED C-PEPTIDE”, which is incorporated herein by reference.

A search of patents, published patent applications, and related publications will also provide those skilled in the art reading this disclosure with significant possible PEG-coupling technologies and PEG-derivatives. For example, U.S. Pat. Nos. 6,436,386; 5,932,462; 5,900,461; 5,824,784; and 4,904,584; the contents of which are incorporated by reference in their entirety, describe such technologies and derivatives, and methods for their manufacture. Thus, one skilled in the art, considering both the disclosure of this invention and the disclosures of these other patents could couple PEG, a PEG-derivative, or some other polymer to C-peptide for its extended release.

PEG is a well-known polymer having the properties of solubility in water and in many organic solvents, lack of toxicity, lack of immunogenicity, and also clear, colorless, odorless, and stable. One use of PEG is to covalently attach the polymer to insoluble molecules to make the resulting PEG-molecule conjugate soluble. For these reasons and others, PEG has been selected as the preferred polymer for attachment, but it has been employed solely for purposes of illustration and not limitation. Similar products may be obtained with other water soluble polymers, including without limitation; polyvinyl alcohol, other poly(alkylene oxides) such as poly(propylene glycol) and the like, poly(oxyethylated polyols) such as poly(oxyethylated glycerol) and the like, carboxymethylcellulose, dextran, polyvinyl alcohol, polyvinyl purrolidone, poly-1,3-dioxolane, poly-1,3,6-trioxane, ethylene/maleic anhydride, and polyaminoacids. One skilled in the art will be able to select the desired polymer based on the desired dosage, circulation time, resistance to proteolysis, and other considerations.

Isomers of the native L-amino acids, e.g., D-amino acids may be incorporated in any of the above forms of C-peptide, and used in any of the methods of the invention. Additional variants may include amino and/or carboxyl terminal fusions as well as intrasequence insertions of single or multiple amino acids. Longer peptides may comprise multiple copies of one or more of the C-peptide sequences, such as any of Seq ID Nos. 1-32. Insertional amino acid sequence variants are those in which one or more amino acid residues are introduced at a site in the protein. Deletional variants are characterized by the removal of one or more amino acids from the sequence. Variants may include, e.g., different allelic variants as they appear in nature, e.g., in other species or due to geographical variation. All such variants, derivatives, fusion proteins, or fragments of C-peptide are included, may be used in any of the methods claims disclosed herein, and are subsumed under the term “C-peptide”.

The variants, derivatives, and fragments are functionally equivalent in that they have detectable C-peptide activity. More particularly, they exhibit at least 40%, preferably at least 60%, more preferably at least 80% of the activity of proinsulin C-peptide, particularly human C-peptide. Thus they are capable of functioning as proinsulin C-peptide, i.e., can substitute for C-peptide itself. Such activity means any activity exhibited by a native C-peptide, whether a physiological response exhibited in an in vivo or in vitro test system, or any biological activity or reaction mediated by a native C-peptide, e.g., in an enzyme assay or in binding to test tissues, membranes, or metal ions. Thus, it is known that C-peptide increases the intracellular concentration of calcium. An assay for C-peptide activity can thus be made by assaying for changes in intracellular calcium concentrations upon addition or administration of the peptide (e.g., fragment or derivative) in question. Such an assay is described in, e.g., Ohtomo Y et al. (Diabetologia 39: 199-205, (1996)), Kunt T et al. (Diabetologia 42(4): 465-471, (1999)), Shafqat J et al. (Cell Mol. Life. Sci. 59: 1185-1189, (2002)). Further, C-peptide has been found to induce phosphorylation of the MAP-kinases ERK 1 and 2 of a mouse embryonic fibroblast cell line (Swiss 3T3), and measurement of such phosphorylation and MAPK activation may be used to assess, or assay for C-peptide activity, as described, e.g., by Kitamura T et al. (Biochem. J. 355: 123-129, (2001)). C-peptide also has a well-known effect in stimulating Na⁺K⁺-ATPase activity and this also may form the basis of an assay for C-peptide activity, e.g., as described in WO 98/13384 or in Ohtomo Y et al. (supra) or Ohtomo Y et al. (Diabetologia 41: 287-291, (1998)). An assay for C-peptide activity based on endothelial nitric oxide synthase (eNOS) activity is also described in Kunt T et al. (supra) using bovine aortic cells and a reporter cell assay. Binding to particular cells may also be used to assess or assay for C-peptide activity, e.g., to cell membranes from human renal tubular cells, skin fibroblasts, and saphenous vein endothelial cells using fluorescence correlation spectroscopy, as described, e.g., in Rigler R et al. (PNAS USA 96: 13318-13323, (1999)), Henriksson M et al. (Cell Mol. Life. Sci. 57: 337-342, (2000)) and Pramanik A et al. (Biochem Biophys. Res. Commun. 284: 94-98, (2001)).

### C-Peptide Therapeutic Dose Forms

Human C-peptide may be produced by recombinant technology, e.g., as a by-product in the production of human insulin from human proinsulin, or using genetically modified E. coli (see WO 1999007735) or synthetically using standard solid-phase peptide synthesis.

Administration of a therapeutic dose of C-peptide may be by any suitable method known in the medicinal arts, including oral, parenteral, topical, or subcutaneous administration, inhalation, or the implantation of a sustained delivery device or composition. In one aspect, administration is by subcutaneous administration. The C-peptide may be administered at any time during the day. For humans, the daily dosage used may range from about 0.1 to 10 mg/24 hours of C-peptide, e.g., from about 0.1 to 0.3 mg, about 0.3 to 1.5 mg, about 1.5 to 2.25 mg, about 2.25 to 3.0 mg, about 3.0 to 6.0 mg, and about 6.0 to 10 mg/24 hours. Preferably the total daily dose used is about 0.45 to 0.9 mg, about 0.6 to 1.2 mg, about 1.2 to 2.4 mg, or about 2.5 to 3.0 mg/24 hours. The total daily dose may be about 0.3 mg, about 0.45 mg, about 0.6 mg, about 0.9 mg, about 1.2 mg, about 1.5 mg, about 1.8 mg, about 2.1 mg, about 2.4 mg, about 2.7 mg, about 3.0 mg, about 3.3 mg, about 3.6 mg, about 3.9 mg, about 4.2 mg, or about 4.5 mg/24 hours. (It will be appreciated that masses of C-peptide referred to above are dependent on the bioavailability of the delivery system and based on the use of C-peptide with a molecular mass of approximately 3,020 Da).

It will be further appreciated that for sustained delivery devices and compositions the total dose of C-peptide contained in such delivery system will be correspondingly larger depending upon the release profile of the sustained release system. Thus, a sustained release composition or device that is intended to deliver C-peptide over a period of 5 days will typically comprise at least about 5 to 10 times the daily dose of C-peptide; a sustained release composition or device that is intended to deliver C-peptide over a period of 365 days will typically comprise at least about 400 to 800 times the daily dose of C-peptide (depending upon the stability and bioavailability of C-peptide when administered using the sustained release system).

In one aspect of any of these modes of administration, the total daily dose of C-peptide may be administered in multiple, single doses throughout the day to maintain the steady state level of C-peptide above the minimum effective therapeutic level. The size of the single dose as administered will vary depending on the frequency of administration and bioavailability, but may typically be in the region of about 0.15 to 6.0 mg, about 0.15 to 4.5 mg, about 0.15 to 3.0 mg, about 0.15 to 2.4 mg, about 0.15 to 1.8 mg, or about 0.15 to 1.2 mg. Other ranges include about 0.1 to 4.5 mg, about 0.3 to 0.6 mg, about 0.3 to 1.5 mg, or about 0.5 to 3.0 mg. Representative single doses include about 5.0 mg, about 4.5 mg, about 4.0 mg, about 3.5 mg, about 3.0 mg, about 2.5 mg, about 2.0 mg, about 1.5 mg, about 1.0 mg, or about 0.5 mg. In one aspect, the dosing interval of such multiple administration regimens will be about 3 hours between doses, or about 4 hours between doses, or about 6 hours between doses.

In one aspect of any of these methods, the dose and dosing interval of C-peptide administered may vary depending on the time of administration. For example, a total daily dose of 1.8 mg/24 hours may be divided into 4 doses; 0.45 mg in the morning (06:00-10:00); at lunch (11:00-14:00); at dinner (16:00-19:00); and 0.9 mg at bedtime (20:00-24:00). Typically such dosing schedules maintain the average steady-state C-peptide level in the blood above the minimum effective therapeutic level for at least 50% of the time for any one 24 hour dosing period. In a preferred aspect, the dosing schedule maintains the C-peptide level in the blood above the minimum effective therapeutic level for at least 75% of the time for any one 24 hour dosing period. In a more preferred aspect, the dosing schedule maintains the C-peptide level in the blood above the minimum effective therapeutic level for at least 85% of the time for any one 24 hour dosing period. In another aspect of any of these modes of administration, the total daily dose of C-peptide may be administered continuously throughout the day to coordinate C-peptide levels with insulin levels, meals, or periods of exercise, sleep, or any other patient-specific clinical parameter or biomarker.

The therapeutic dose of C-peptide may or may not be in solution. If the dose is administered in solution, it will be appreciated that the volume of the dose may vary, but will typically be 10 μL-2 mL. Preferably the dose for S.C. administration will be given in a volume of 1000 uL, 900 μL, 800 μL, 700 μL, 600 μL, 500 μL, 400 μL, 300 μL, 200 μL, 100 μL, 50 μL, or 20 μL. Sustained release compositions and depot formulations may include doses in volumes of about 2 mL to about 50 uL.

C-peptide doses in solution can also comprise a preservative and/or a buffer. For example, the preservative m-cresol can be used. Typical concentrations of preservatives include 0.5 mg/mL, 1 mg/mL, 2 mg/mL, 3 mg/mL, 4 mg/mL, or 5 mg/mL. Thus, a range of concentration of preservative may include 0.2 to 10 mg/mL, particularly 0.5 to 6 mg/mL, or 0.5 to 5 mg/mL. Examples of buffers that can be used include, histidine (pH 6.0), sodium phosphate buffer (pH 7.3), or sodium bicarbonate buffer (pH 7.3). It will be appreciated that the C-peptide dose may comprise one or more of a native or intact C-peptide, fragments, derivatives, or other functionally equivalent variants of C-peptide.

A therapeutic dose of C-peptide may comprise full-length human C-peptide (SEQ. ID. NO. 1) and the C-terminal C-peptide fragment EGSLQ (SEQ. ID. NO. 31) and/or a C-peptide homolog or C-peptide derivative. Further, the dose may if desired only contain a fragment of C-peptide, e.g., EGSLQ. Thus, the term “C-peptide” may encompass a single C-peptide entity or a mixture of different “C-peptides”.

Pharmaceutical compositions for use in the present invention may be formulated according to techniques and procedures well-known in the art and widely discussed in the literature and may comprise any of the known carriers, diluents, or excipients. In one aspect, the compositions may be in the form of (sterile) aqueous solutions and/or suspensions of the pharmaceutically active ingredients, aerosols, ointments, and the like. Formulations which are aqueous solutions are most preferred. Such formulations typically contain the C-peptide itself, water, and one or more buffers which act as stabilizers (e.g., phosphate-containing buffers) and optionally one or more preservatives. Such formulations containing, e.g., about 0.3 to 12.0 mg, about 0.3 to 10.0 mg, about 0.3 to 8 mg, about 0.3 to 6.0 mg, about 0.3 to 4.0 mg, about 0.3 to 3.0 mg, or any of the ranges mentioned above, e.g., about 12 mg, about 10 mg, about 8 mg, about 6 mg, about 5 mg, about 4 mg, about 3 mg, about 2 mg, or about 1 mg of the C-peptide and constitute a further aspect of the invention.

Pharmaceutical compositions may include pharmaceutically acceptable salts of C-peptide. For a review on suitable salts, see Handbook of Pharmaceutical Salts: Properties, Selection, and Use by Stahl and Wermuth (Wiley-VCH, 2002). Suitable base salts are formed from bases which form non-toxic salts. Representative examples include the aluminium, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts. In one embodiment, C-peptide may be prepared as a gel with a pharmaceutically acceptable positively charged ion. In one aspect, the positively charged ion may be a divalent metal ion. In one aspect, the metal ion is selected from calcium, magnesium, and zinc.

Compositions to be used in the invention suitable for parenteral administration may comprise sterile aqueous solutions and/or suspensions of the pharmaceutically active ingredients preferably made isotonic with the blood of the recipient, generally using sodium chloride, glycerin, glucose, mannitol, sorbitol, and the like.

Compositions of the invention suitable for oral administration may, e.g., comprise peptides in sterile purified stock powder form preferably covered by an envelope or envelopes (enterocapsules) protecting from degradation of the peptides in the stomach and thereby enabling absorption of these substances from the gingiva or in the small intestines. The total amount of active ingredient in the composition may vary from 99.99 to 0.01 percent of weight.

Methods for Administration of C-Peptide

Pharmaceutical compositions suitable for the delivery of C-peptide and methods for their preparation will be readily apparent to those skilled in the art. Such compositions and methods for their preparation may be found, e.g., in Remington's Pharmaceutical Sciences, 19th Edition (Mack Publishing Company, 1995).

Pharmaceutical compositions of C-peptide may be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular, intrasynovial, and subcutaneous. Suitable devices for parenteral administration include needle (including microneedle) injectors, needle-free injectors, and infusion techniques. Subcutaneous administration of C-peptide is preferred. Subcutaneous administration of C-peptide will typically not be into the same site as that most recently used for insulin administration. In one aspect of any of the claimed methods, C-peptide is administered to the opposite side of the abdomen to the site most recently used for insulin administration. In another aspect of any of the claimed methods, C-peptide is administered to the upper arm. In another aspect of any of the claimed methods, C-peptide is administered to the abdomen. In another aspect of any of the claimed methods, C-peptide is administered to the upper area of the buttock. In another aspect of any of the claimed methods, C-peptide is administered to the front of the thigh.

Parenteral formulations are typically aqueous solutions which may contain excipients such as salts, carbohydrates, and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, e.g., by lyophilization, may readily be accomplished using standard pharmaceutical techniques well-known to those skilled in the art.

Formulations for parenteral administration may be formulated to be immediate and/or sustained release. Sustained release compositions include delayed, modified, pulsed, controlled, targeted and programmed release. Thus C-peptide may be formulated as a suspension or as a solid, semi-solid, or thixotropic liquid for administration as an implanted depot providing sustained release of C-peptide. Examples of such formulations include without limitation, drug-coated stents and semi-solids and suspensions comprising drug-loaded poly(DL-lactic-co-glycolic)acid (PGLA), poly(DL-lactide-co-glycolide) (PLG) or poly(lactide) (PLA) lamellar vesicles or microparticles, hydrogels (Hoffman A S: Ann. N.Y. Acad. Sci. 944: 62-73 (2001)), poly-amino acid nanoparticles systems, such as the Medusa system developed by Flamel Technologies Inc., non aqueous gel systems such as Atrigel developed by Atrix, Inc., and SABER (Sucrose Acetate Isobutyrate Extended Release) developed by Durect Corporation, and lipid-based systems such as DepoFoam developed by SkyePharma. In another embodiment of the present invention, the sustained release of C-peptide into the blood comprises a sustained release composition comprising PEGylated C-peptide that is injected subcutaneously.

Sustained release devices capable of delivering desired doses of C-peptide over extended periods of time are known in the art. For example, U.S. Pat. Nos. 5,034,229; 5,557,318; 5,110,596; 5,728,396; 5,985,305; 6,113,938; 6,156,331; 6,375,978; and 6,395,292; teach osmotically-driven devices capable of delivering an active agent formulation, such as a solution or a suspension, at a desired rate over an extended period of time (i.e., a period ranging from more than one week up to one year or more). Other exemplary sustained release devices include regulator-type pumps that provide constant flow, adjustable flow, or programmable flow of beneficial agent formulations, which are available from, e.g., OmniPod™ Insulin Management System (Insulet Corporation, Codman of Raynham, Mass., Medtronic of Minneapolis, Minn., Intarcia Therapeutics of Hayward, Calif., and Tricumed Medinzintechnik GmbH of Germany. Further examples of devices are described in U.S. Pat. Nos. 6,283,949; 5,976,109; 5,836,935; and 5,511,355.

Generally, in an osmotic pump system, a core is encased by a semi-permeable membrane having at least one orifice. The semi-permeable membrane is permeable to water, but impermeable to the active agent. When the system is exposed to body fluids, water penetrates through the semi-permeable membrane into the core containing osmotic excipients and the active agent. Osmotic pressure increases within the core and the agent is displaced through the orifice at a controlled, predetermined rate.

In many osmotic pumps, the core contains more than one internal compartment. For example, a first compartment may contain the active agent. A second compartment contains an osmotic agent and/or “driving member.” See, e.g., U.S. Pat. No. 5,573,776, the contents of which are incorporated herein by reference. This compartment may have a high osmolality, which causes water to flux into the pump through the semi permeable membrane. The influx of water compresses the first compartment. This can be accomplished, e.g., by using a polymer in the second compartment, which swells on contact with the fluid. Accordingly, the agent is displaced at a predetermined rate.

In another embodiment, the osmotic pump may comprise more than one active agent-containing compartment, with each compartment containing the same agent or a different agent. The concentrations of the agent in each compartment, as well as the rate of release, may also be the same or different.

The rate of delivery is generally controlled by the water permeability of the semi-permeable membrane. Thus, the delivery profile of the pump is independent of the agent dispensed, and the molecular weight of an agent, or its physical and chemical properties, generally have no bearing on its rate of delivery. Further discussion regarding the principle of operation, the design criteria, and the delivery rate for osmotic pumps is provided in Theeuwes and Yum (Ann. of Biomed. Eng. 4(4): 343-353, (1976)) and Urquhart J et al. (Ann. Rev. Pharmacol. Toxicol. 24:199-236, (1984)), the contents of which are incorporated by reference.

Sustained release devices based on osmotic pumps are well-known in the art and readily available to one of ordinary skill in the art from companies experienced in providing osmotic pumps for extended release drug delivery. For example, the technology sold under the trademark DUROS®, which was originally developed by ALZA, is an implantable, nonbiodegradable, osmotically-driven system that enables delivery of small drugs, peptides, proteins, DNA, and other bioactive macromolecules for up to one year; ALZA's technology sold under the trademark OROS® embodies tablets that employ osmosis to provide precise, controlled drug delivery for up to 24 hours; Osmotica Pharmaceutical's Osmodex® system includes a tablet, which may have more than one layer of the drug(s) with the same or different release profiles; Shire Laboratories' EnSoTrol® system solubilizes drugs within the core and delivers the solubilized drug through a laser-drilled hole by osmosis; and ALZET® osmotic pumps are miniature, implantable pumps used for research in mice, rats, and other laboratory animals.

A search of patents, published patent applications, and related publications will also provide those skilled in the art reading this disclosure with significant possible osmotic pump technologies. For example, U.S. Pat. Nos. 6,890,918; 6,838,093; 6,814,979; 6,713,086; 6,534,090; 6,514,532; 6,361,796; 6,352,721; 6,294,201; 6,284,276; 6,110,498; 5,573,776; 4,200,0984; and 4,088,864; the contents of which are incorporated herein by reference, describe osmotic pumps and methods for their manufacture. One skilled in the art, considering both the disclosure of this invention and the disclosures of these other patents could produce an osmotic pump for the sustained release of C-peptide.

Typical materials for the semi-permeable membrane include semi-permeable polymers known to the art as osmosis and reverse osmosis membranes, such as cellulose acylate, cellulose diacylate, cellulose triacylate, cellulose acetate, cellulose diacetate, cellulose triacetate, agar acetate, amylase triacetate, beta glucan acetate, acetaldehyde dimethyl acetate, cellulose acetate ethyl carbamate, polyamides, plyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate, succinate, methyl cellulose, cellulose acetate p-toluene sulfonate, cellulose acetate butyrate, cross-linked selectively semi-permeable polymers formed by the coprecipitation of a polyanion and a polycation, semi-permeable polymers, lightly cross-linked polystyrene derivatives, cross-linked poly(sodium styrene sulfonate), poly(vinylbenzyltrimethyl ammonium chloride), cellulose acetate having a degree of substitution up to 1 and an acetyl content up to 50%, cellulose diacetate having a degree of substitution of 1 to 2 and an acetyl content of 21 to 35%, cellulose triacetate having a degree of substitution of 2 to 3 and an acetyl content of 35 to 44.8%, as disclosed in U.S. Pat. No. 6,713,086, the contents of which are incorporated herein by reference.

The osmotic agent(s) present in the pump may comprise any osmotically effective compound(s) that exhibit an osmotic pressure gradient across the semi-permeable wall against the exterior fluid. Effective agents include, without limitation, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, flucose, hydrophilic polymers such as cellulose polymers, mixtures thereof, and the like, as disclosed in U.S. Pat. No. 6,713,086, the contents of which are incorporated herein by reference.

The “driving member” is typically a hydrophilic polymer that interacts with biological fluids and swells or expands. The polymer exhibits the ability to swell in water and retain a significant portion of the imbibed water within the polymer structure. The polymers swell or expand to a very high degree, usually exhibiting a 2- to 50-fold volume increase. The polymers can be non-cross-linked or cross-linked. Hydrophilic polymers suitable for the present purpose are well-known in the art.

The orifice may comprise any means and methods suitable for releasing the active agent from the system. The osmotic pump may include one or more apertures or orifices that have been bored through the semi-permeable membrane by mechanical procedures known in the art, including, but not limited to, the use of lasers as disclosed in U.S. Pat. No. 4,088,864. Alternatively, it may be formed by incorporating an erodible element, such as a gelatin plug, in the semi-permeable membrane.

Because they can be designed to deliver a desired active agent at therapeutic levels over an extended period of time, implantable delivery systems can advantageously provide long-term therapeutic dosing of a desired active agent without requiring frequent visits to a healthcare provider or repetitive self-medication. Therefore, implantable delivery devices can work to provide increased patient compliance, reduced irritation at the site of administration, fewer occupational hazards for healthcare providers, reduced waste hazards, and increased therapeutic efficacy through enhanced dosing control.

Among other challenges, two problems must be addressed when seeking to deliver biomolecular material over an extended period of time from an implanted delivery device. First, the biomolecular material must be contained within a formulation that substantially maintains the stability of the material at elevated temperatures (i.e., 37° C. and above) over the operational life of the device. Second, the biomolecular material must be formulated in a way that allows delivery of the biomolecular material from an implanted device into a desired environment of operation over an extended period time. This second challenge has proven particularly difficult where the biomolecular material is included in a flowable composition that is delivered from a device over an extended period of time at low flow rates (i.e., ≦100 μL/day).

Peptide drugs such as C-peptide may degrade via one or more of several different mechanisms, including deamidation, oxidation, hydrolysis, and racemization. Significantly, water is a reactant in many of the relevant degradation pathways. Moreover, water acts as a plasticizer and facilitates the unfolding and irreversible aggregation of biomolecular materials. To work around the stability problems created by aqueous formulations of biomolecular materials, dry powder formulations of biomolecular materials have been created using known particle formation processes, such as by known lyophilization, spray-drying, or desiccation techniques. Though dry powder formulations of biomolecular material have been shown to provide suitable stability characteristics, it would be desirable to provide a formulation that is not only stable over extended periods of time, but is also flowable and readily deliverable from an implantable delivery device.

Accordingly in one aspect of any of the claimed methods, the C-peptide is provided in a non-aqueous drug formulation, and is delivered from a sustained release implantable device, wherein the C-peptide is stable for at least two months of time at 37° C.

Representative non-aqueous formulations for C-peptide include those disclosed in International Publication Number WO00/45790 that describes nonaqueous vehicle formulations that are formulated using at least two of a polymer, a solvent, and a surfactant.

WO98/27962 discloses an injectable depot gel composition containing a polymer, a solvent that can dissolve the polymer and thereby form a viscous gel, a beneficial agent, and an emulsifying agent in the form of a dispersed droplet phase in the viscous gel.

WO04089335 discloses nonaqueous vehicles that are formed using a combination of polymer and solvent that results in a vehicle that is miscible in water. As it is used herein, the term “miscible in water” refers to a vehicle that, at a temperature range representative of a chosen operational environment, can be mixed with water at all proportions without resulting in a phase separation of the polymer from the solvent such that a highly viscous polymer phase is formed. For the purposes of the present invention, a “highly viscous polymer phase” refers to a polymer containing composition that exhibits a viscosity that is greater than the viscosity of the vehicle before the vehicle is mixed with water.

Accordingly in another aspect of any of the claimed methods and, C-peptide is provided in a sustained release device comprising: a reservoir having at least one drug delivery orifice, and a stable non-aqueous drug formulation. In one aspect of these methods, the formulation comprises: at least C-peptide; and a non-aqueous, single-phase vehicle comprising at least one polymer and at least one solvent, the vehicle being miscible in water, wherein the drug is insoluble in one or more vehicle components and the C-peptide formulation is stable at 37° C. for at least two months. In one aspect, the solvent is selected from the group consisting of glycofurol, benzyl alcohol, tetraglycol, n-methylpyrrolidone, glycerol formal, propylene glycol, and combinations thereof.

In particular, a non-aqueous formulation is considered chemically stable if no more than about 35% of the C-peptide is degraded by chemical pathways, such as by oxidation, deamidation, and hydrolysis, after maintenance of the formulation at 37° C. for a period of two months, and a formulation is considered physically stable if, under the same conditions, no more than about 15% of the C-peptide contained in the formulation is degraded through aggregation. A drug formulation is stable according to the present invention if at least about 65% of the C-peptide remains physically and chemically stable after about two months at 37° C.

C-peptide for use in the present invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages, and microemulsions. Liposomes may also be used. Typical carriers include alcohol, water, mineral oil, liquid petrolatum, white petrolatum, glycerin, polyethylene glycol, and propylene glycol. Penetration enhancers may be incorporated—see, e.g., Finnin and Morgan: J. Pharm. Sci. 88(10): 955-958, (1999). Other means of topical administration include delivery by electroporation, iontophoresis, phonophoresis, sonophoresis, and microneedle or needle-free injection (e.g., products sold under the trademarks POWDERJECT™ and BIOJECT™).

Formulations for topical administration may be formulated to be immediate and/or modified release. Modified release formulations include delayed, sustained, pulsed, controlled, targeted and programmed release.

In another embodiment of a sustained release composition of C-peptide, the C-peptide is packaged in a liposome, which has demonstrated utility in delivering beneficial active agents in a controlled manner over prolonged periods of time. Liposomes are completely closed bilayer membranes containing an entrapped aqueous volume. Liposomes may be unilamellar vesicles possessing a single membrane bilayer or multilamellar vesicles with multiple membrane bilayers, each separated from the next by an aqueous layer. The structure of the resulting membrane bilayer is such that the hydrophobic (non-polar) tails of the lipid orient toward the center of the bilayer while the hydrophilic (polar) heads orient towards the aqueous phase.

Generally, in a liposome-drug delivery system, the active agent is entrapped in the liposome and then administered to the patient to be treated. However, if the active agent is lipophilic, it may associate with the lipid bilayer. The immune system may recognize conventional liposomes as foreign bodies and destroy them before significant amounts of the active agent reaches the intended disease site. Thus, in one embodiment, the liposome may be coated with a flexible water-soluble polymer that avoids uptake by the organs of the mononuclear phagocyte system, primarily the liver and spleen. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose hydroxyethylcellulose, polyethyleneglycol, polyaspartamide and hydrophilie peptide sequences as described in U.S. Pat. Nos. 6,316,024; 6,126,966; 6,056,973; 6,043,094; the contents of which are incorporated by reference in their entirety.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phasphatidylglycerol, phosphatidylinositol, and sphingomyelin as disclosed in U.S. Pat. Nos. 6,056,973 and 5,874,104. The vesicle-forming lipids may also be glycolipids, cerebrosides, or cationic lipids, such as 1,2-dioleyloxy-3-(trimethylamino) propane (DOTAP); N-;I-(2,3,-ditetradecyloxy)propyl;-N,N-dimethyl-N-hydroxyethylammonium bromide (DMRIE); N 2,3,-dioleyloxy)propyl; N,N-dimethyl-N-hydroxy ethylammonium bromide (DORIE); N;I-(2,3-dioleyloxy)propyl N,N,N-trimethylammonium chloride (DOTMA); 3;N—(N′,N′-dimethylaminoethane) carbamoly; cholesterol (DC-Choi); or dimethyldioctadecylamnionium (DDAB) also as disclosed in U.S. Pat. No. 6,056,973. Cholesterol may also be present in the proper range to impart stability to the vesicle as disclosed in U.S. Pat. Nos. 5,916,588 and 5,874,104.

The liposomes for use in any of the methods of the invention can be manufactured by standard techniques known to those of skill in the art. For example, in one embodiment, as disclosed in U.S. Pat. No. 5,916,588, a buffered solution of the active agent is prepared. Then a suitable lipid, such as hydrogenated soy phosphatidylcholine, and cholesterol, both in powdered form, are dissolved in chloroform or the like and dried by rotoevaporation. The lipid film thus formed is resuspended in diethyl ether or the like and placed in a flask, and sonicated in a water bath during addition of the buffered solution of the active agent. Once the ether has evaporated, sonication is discontinued and a stream of nitrogen is applied until residual ether is removed. Other standard manufacturing procedures are described in U.S. Pat. Nos. 6,352,716; 6,294,191; 6,126,966; 6,056,973; 5,965,156; and 5,874,104. The liposomes of this invention can be produced by any method generally accepted in the art for making liposomes, including, without limitation, the methods of the above-cited documents (the contents of which are incorporated herein by reference).

Liposomes are also well-known in the art and readily available from companies experienced in providing liposomes for extended release drug delivery. For example, ALZA's (formerly Sequus Pharmaceutical's) liposomal technology sold under the trademark STEALTH® for intravenous drug delivery uses a polyethylene glycol coating on liposomes to evade recognition by the immune system; Gilead Sciences (formerly Nexstar's) liposomal technology was incorporated into AmBisome®, and FDA approved treatment for fungal infections; and NOF Corp. offers a wide variety of GMP-grade phospholipids, phospholipids derivatives, and PEG-phospholipids sold under the trade names COATSOME® and SUNBRIGHT®.

A search of patents, published patent applications, and related publications will also provide those skilled in the art reading this disclosure with significant possible liposomal technologies. U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479; the contents of which are incorporated herein by reference, describe liposomes and lipid-coated microbubbles, and methods for their manufacture. Thus, one skilled in the art, considering both the disclosure of this invention and the disclosures of these other patents could produce a liposome for the sustained release of C-peptide.

In another embodiment of the present invention, the sustained release of C-peptide into the blood comprises a sustained release composition comprising C-peptide that is packaged in a microsphere. Microspheres have demonstrated utility in delivering beneficial active agents to a target area in a controlled manner over prolonged periods of time. Microspheres are generally biodegradable and can be used for subcutaneous, intramuscular, and intravenous administration.

Generally, each microsphere is composed of an active agent and polymer molecules as disclosed in U.S. Pat. No. 6,268,053, the active agent may be centrally located within a membrane formed by the polymer molecules, or, alternatively, dispersed throughout the microsphere because the internal structure comprises a matrix of the active agent and a polymer excipient. Typically, the outer surface of the microsphere is permeable to water, which allows aqueous fluids to enter the microsphere, as well as solubilized active agent and polymer to exit the microsphere.

In one embodiment, the polymer membrane comprises cross-linked polymers as disclosed in U.S. Pat. No. 6,395,302. When the pore sizes of the cross-linked polymer are equal or smaller than the hydrodynamic diameter of the active agent, the active agent is essentially released when the polymer is degraded. On the other hand, if the pore sizes of the cross-linked polymers are larger than the size of the active agent, the active agent is at least partially released by diffusion.

Additional methods for making microsphere membranes are known and used in the art and can be used in the practice of the invention disclosed herein. Typical materials for the outer membrane include the following categories of polymers: (1) carbohydrate-based polymers, such as methylcellulose, carboxymethyl cellulose-based polymers, dextran, polydextrose, chitins, chitosan, and starch (including hetastarch), and derivatives thereof; (2) polyaliphatic alcohols such as polyethylene oxide and derivatives thereof including polyethylene glycol (PEG), PEG-acrylates, polyethyleneimine, polyvinyl acetate, and derivatives thereof; (3) polyvinyl polymers such as polyvinyl alcohol, polyvinylpyrrolidone, poly(vinyl)phosphate, poly(vinyl)phosphonic acid, and derivatives thereof; (4) polyacrylic acids and derivatives thereof; (5) polyorganic acids, such as polymaleic acid, and derivatives thereof; (6) polyamino acids, such as polylysine, and poly-imino acids, such as polyimino tyrosine, and derivatives thereof; (7) co-polymers and block co-polymers, such as poloxamer 407 or Pluronic L-101; polymer, and derivatives thereof; (8) tert-polymers and derivatives thereof; (9) polyethers, such as poly(tetramethylene ether glycol), and derivatives thereof; (10) naturally-occurring polymers, such as zein, chitosan and pullulan, and derivatives thereof; (11) polyimids, such as poly n-tris(hydroxymethyl) methylmethacrylate, and derivatives thereof; (12) surfactants, such as polyoxyethylene sorbitan, and derivatives thereof; (13) polyesters such polyethylene glycol) (n) monomethyl ether mono(succinimidyl succinate)ester, and derivatives thereof; (14) branched and cyclo-polymers, such as branched PEG and cyclodextrins, and derivatives thereof; and (15) polyaldehydes, such as poly(perfluoropropylene oxide-b-perfluoroformaldehyde), and derivatives thereof as disclosed in U.S. Pat. No. 6,268,053, the contents of which are incorporated herein by reference. Other typical polymers known to those of ordinary skill in the art include poly(lactide-co-glycolide), polylactide homopolymer; polyglycolide homopolymer; polycaprolactone; polyhydroxybutyrate-polyhydroxyvalerate copolymer; poly(lactide-co-caprolactone); polyesteramides; polyorthoesters; poly 13-hydroxybutyric acid; and polyanhydrides as disclosed in U.S. Pat. No. 6,517,859, the contents of which are incorporated herein by reference.

In one embodiment, the microsphere of the present invention are attached to or coated with additional molecules. Such molecules can facilitate targeting, enhance receptor mediation, and provide escape from endocytosis or destruction. Typical molecules include phospholipids, receptors, antibodies, hormones, and polysaccharides. Additionally, one or more cleavable molecules may be attached to the outer surface of microspheres to target it to a predetermined site. Then, under appropriate biological conditions, the molecule is cleaved causing release of the microsphere from the target.

The microspheres for use in the sustained release compositions are manufactured by standard techniques. For example, in one embodiment, volume exclusion is performed by mixing the active agent in solution with a polymer or mixture of polymers in solution in the presence of an energy source for a sufficient amount of time to form particles as disclosed in U.S. Pat. No. 6,268,053. The pH of the solution is adjusted to a pH near the isoelectric point (pi) of the macromolecule. Next, the solution is exposed to an energy source, such as heat, radiation, or ionization, alone or in combination with sonication, vortexing, mixing or stirring, to form microparticles. The resulting microparticles are then separated from any unincorporated components present in the solution by physical separation methods well-known to those skilled in the art and may then be washed. Other standard manufacturing procedures are described in U.S. Pat. Nos. 6,669,961; 6,517,859; 6,458,387; 6,395,302; 6,303,148; 6,268,053; 6,090,925; 6,024,983; 5,942,252; 5,981,719; 5,578,709; 5,554,730; 5,407,609; 4,897,268; and 4,542,025; the contents of which are incorporated by reference in their entirely. Microspheres are well-known and readily available to one of ordinary skill in the art from companies experienced in providing such technologies for extended release drug delivery. For example, Epic Therapeutics, a subsidiary of Baxter Healthcare Corp., developed a protein-matrix drug delivery system sold under the trademark PROMAXX®, that produces bioerodible protein microspheres in a totally water-based process; OctoPlus developed a cross-linked dextran microsphere sold under the trademark OctoDEX®, that release active ingredients based on bulk degradation of matrix rather than based on surface erosion.

A search of patents, published patent applications, and related publications will also provide those skilled in the art reading this disclosure with significant possible microsphere technologies for use in formulating sustained release compositions. For example, U.S. Pat. Nos. 6,669,961; 6,517,859; 6,458,387; 6,395,302; 6,303,148; 6,268,053; 6,090,925; 6,024,983; 5,942,252; 5,981,719; 5,578,709; 5,554,730; 5,407,609; 4,897,268; and 4,542,025; the contents of which are incorporated by reference in their entirety, describe microspheres and methods for their manufacture. One skilled in the art, considering both the disclosure of this invention and the disclosures of these other patents could make and use microspheres for the sustained release of C-peptide for use in any of the methods claimed herein.

The C-peptide can be administered intranasally or by inhalation, typically in the form of a dry powder (either alone, as a mixture, e.g., in a dry blend with lactose, or as a mixed component particle, e.g., mixed with phospholipids, such as phosphatidylcholine) from a dry powder inhaler, as an aerosol spray from a pressurized container, pump, spray, atomizer (preferably an atomizer using electro hydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, e.g., chitosan or cyclodextrin.

The pressurized container, pump, spray, atomizer, or nebulizer contains a solution or suspension of the compound(s) of the invention comprising, e.g., ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate method, such as spiral jet milling, fluid bed jet milling, supercritical fluid processing to form nanoparticles, high pressure homogenization, or spray drying.

Capsules (made, e.g., from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation for use in an atomizer using electro hydrodynamics to produce a fine mist may contain from 1 μg to 20 mg of C-peptide per actuation and the actuation volume may vary from 1 μL to 100 μL. A typical formulation may comprise C-peptide propylene glycol, sterile water, ethanol, and sodium chloride. Alternative solvents that may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, e.g., PGLA. Modified release formulations include delayed, sustained, pulsed, controlled, targeted and programmed release.

In the case of dry powder inhalers and aerosols, the dosage unit is determined by means of a valve that delivers a metered amount. Units in accordance with the invention are typically arranged to administer a metered dose or “puff” containing from 0.1 mg to 10 mg of C-peptide. The overall daily dose will typically be in the range 0.1 mg to 20 mg that may be administered in a single dose or, more usually, as divided doses throughout the day.

Combination Therapies

The present invention also includes combination therapies comprising administering to a patient a therapeutic dose of C-peptide, in combination with a second active agent, or a device or a procedure for treating erectile dysfunction. In one aspect of these combination therapies, the second active agent is selected from a type V phosphodiesterase inhibitor, a rho-kinase inhibitor, apomorphine, testosterone undecanoate, testosterone gel and L-arginine. In another aspect, of these combination therapies, the device or procedure is selected from vascular extracorporeal shockwave therapy (Vascuspec) and infrared radiation therapy.

As described above, C-peptide, may be administered in combination with a PDE-5 inhibitor to enhance the effect of the PDE-5 inhibitor. In one aspect of this method, C-peptide is administered to a patient who is unresponsive to the PDE-5 inhibitor. In one aspect the patient has diabetes. In one aspect the patient has insulin-dependent diabetes.

The PDE5 inhibitors useful in this invention may be widely chosen from among any of those already known to the art or subsequently discovered and/or hereafter developed. Suitable PDE5 inhibitors include those disclosed in any of the following US patents, all of which are incorporated herein by reference: a 5-substituted pyrazolo[4,3-d]pyrimidine-7-one as disclosed in U.S. Pat. No. 4,666,908; a griseolic acid derivative as disclosed in any of U.S. Pat. Nos. 4,634,706, 4,783,532, 5,498,819, 5,532,369, 5,556,975, and 5,616,600; a 2-phenylpurinone derivative as disclosed in U.S. Pat. No. 4,885,301; a phenylpyridone derivative as disclosed in U.S. Pat. No. 5,254,571; a fused pyrimidine derivative as disclosed in U.S. Pat. No. 5,047,404; a condensed pyrimidine derivative as disclosed in U.S. Pat. No. 5,075,310; a pyrimidopyrimidine derivative as disclosed in U.S. Pat. No. 5,162,316; a purine compound as disclosed in U.S. Pat. No. 5,073,559; a quinazoline derivative as disclosed in U.S. Pat. No. 5,147,875; a phenylpyrimidone derivative as disclosed in U.S. Pat. No. 5,118,686; an imidazoquinoxalinone derivative or its aza analog as disclosed in U.S. Pat. Nos. 5,055,465 and 5,166,344; a phenylpyrimidone derivative as disclosed in U.S. Pat. No. 5,290,933; a 4-aminoquinazoline derivative as disclosed in U.S. Pat. No. 5,436,233 or 5,439,895; a 4,5-dihydro-4-oxo-pyrrolo[1,2-a]quinoxaline derivative as disclosed in U.S. Pat. No. 5,405,847; a polycyclic guanine derivative as disclosed in U.S. Pat. No. 5,393,755; a nitrogenous heterocyclic compound as disclosed in U.S. Pat. No. 5,576,322; a quinazoline derivative as disclosed in U.S. Pat. No. 4,060,615; and a 6-heterocyclyl pyrazolo[3,4-d]pyrimidin-4-one as disclosed in U.S. Pat. No. 5,294,612. Other disclosures of cGMP PDE inhibitors include the following, all of which are herein incorporated by reference: European patent Application (EPA) publication no. 0428268; European patent 0442204; International patent application publication no. WO 94/19351; Japanese patent application 5-222000; European Journal Of Pharmacology, 251, (1994), 1; and International patent application publication no. WO 94/22855.

In this context “administered in combination” means: (1) part of the same unitary dosage form; (2) administration separately, but as part of the same therapeutic treatment program or regimen, typically but not necessarily, on the same day. Preferably, the C-peptide may be administered at a fixed daily dosage, and the PDE-5 inhibitors taken on an as needed basis, in advance of expected sexual activity, usually not more than once daily.

When C-peptide is administered as adjuvant therapy with a second active agent such as a PDE5 inhibitor, a preferred daily dosage is about 1 mg to about 6 mg/24 hours, more preferably 1.5 mg to 4.5 mg/24 hours. In one aspect the therapeutic dose of C-peptide maintains an average steady state concentration of C-peptide in the patient's plasma above about 0.2 nM. In another aspect the therapeutic dose of C-peptide maintains an average steady state concentration of C-peptide in the patient's plasma above about 0.4 nM, above about 0.6 nM, above about 0.8 nM, or above about 1.0 nM.

The routes of administration of the second active agent can be any of those known to the art such as oral, parenteral via local injection intracavernosally or intraurethrally, or transdermal as by applying the active component in a gel or other such formulation topically to the penis. The second active agent can be formulated as known in the art, usually together with a pharmaceutically acceptable carrier or diluent, for example as a tablet, capsule, lozenge, troche, elixir, solution, or suspension for oral administration, in a suitable injectable vehicle for parenteral administration, or as a lotion, ointment or cream for topical application.

The exact dose of each component administered will, of course, differ depending on the specific components prescribed, on the subject being treated, on the severity of the impotence, on the manner of administration and on the judgment of the prescribing physician. Thus, because of patient-to-patient variability, the dosages given below are a guideline and the physician may adjust doses of the compounds to achieve the treatment that the physician considers appropriate for the patient, male. In considering the degree of treatment desired, the physician must balance a variety of factors such as the age of the patient and the presence of other diseases or conditions (e.g., cardiovascular disease). In general, the PDE5 inhibitor will be administered in a range of from 0.5 to 200 mg per day, preferably 10 to 125 mg per day, more preferably 25-100 mg per day. As way of example, and not limitation, a suitable daily oral dosage of PDE-5 inhibitor is in range of 25 to 100 mg for sildenafil; 5 to 20 mg for vardenafil; and 2.5 to 20 mg for tadalafil.

For oral administration a pharmaceutical composition comprising a second active agent can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art. For purposes of transdermal (e.g., topical) administration, dilute sterile, aqueous or partially aqueous solutions (usually in about 0.1% to 5% concentration), otherwise similar to the above parenteral solutions, are prepared. Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

### EXAMPLES

### Example 1

### Demonstration of Improved Sexual Function During C-Peptide Therapy

I Overall Study Design

The study was a multicenter, double-blind, randomized, placebo-controlled phase II trial comparing the effect of subcutaneous injection (S.C.) of 500 nmoL/24 h (1.5 mg) C-peptide; 1,500 nmoL/24 h (4.5 mg) C-peptide and placebo treatment for 6 months in type 1 diabetes patients with peripheral neuropathy.

Five clinical centers participated in this study and patients were recruited to the study by advertisement and by screening of hospital records. Patients who were found eligible and who declared a willingness to participate, were invited to participate in the study and were subsequently screened for inclusion and exclusion criteria.

At the initial screening/baseline visit (S/B visit) the subjects were assigned a screening number (starting with site number 1001). Written informed consent was obtained. Demographic data, medical history, concomitant medication including insulin regimen were recorded in addition to results from a physical examination and electrocardiography (ECG). Furthermore, two neurophysiological examinations were performed within 2-14 days, and neurological examination including symptom assessment were performed. Urine samples and blood samples for hematology, clinical chemistries, and metabolic control (HbA1c) as well as for study specific tests, i.e., C-peptide plasma levels and C-peptide antibodies were also drawn. In women of childbearing potential, pregnancy was excluded by assessment of human chorion gonadotropine (HCG). In addition, autonomic nerve function was assessed by determination of the expiration/inspiration ratio (E/I ratio) during deep breathing. If any information or result of examinations were in violation of the inclusion/exclusion criteria, further assessments were not performed and the patient was discontinued from the study. However, basic demographic data were always recorded.

The eligibility criteria were checked: if a patient did not fulfil the criteria the subject was discontinued from the trial. If the criteria were fulfilled the subject was randomized and given a unique randomization number starting with 6001. Subject was instructed about the trial medication treatment and the first dose was administered at the out-patient clinic under supervision of the study personnel. Instruction and distribution of the diary, trial medication, and other equipment were distributed.

Subsequent clinical visits, visit 1 (1.5 months±2 weeks from S/B visit), visit 2 (3 months±2 weeks from S/B visit) and visit 3 (4.5 months±2 weeks from S/B visit) and final visits, visit 4 (6 months±2 weeks from S/B visit) were scheduled, in relation to the start of study medication.

The second and fourth visits (at 1.5 and at 4.5 months from S/B visit) comprised of a nurse visit including assessment of vital signs. Used and unused clinical trial medication was returned by the patient and drug accountability was performed. The patient diary was reviewed including recording of concomitant medication, adverse events (AEs), and treatment compliance. In addition, a prior-to-dosing C-peptide sample at visit 3 was collected at site 1 (Karolinska Hospital), in 22 patients.

The third visit (at 3 months from S/B visit), comprised HbA1c and safety laboratory tests, and sampling of HLA type, C-peptide antibodies, and C-peptide levels in plasma, physical examination, and review of AEs. Used and unused clinical trial medication was returned by the patient and drug accountability was performed by the study staff. The patient diary was reviewed including recording of concomitant medication, AE, and treatment compliance.

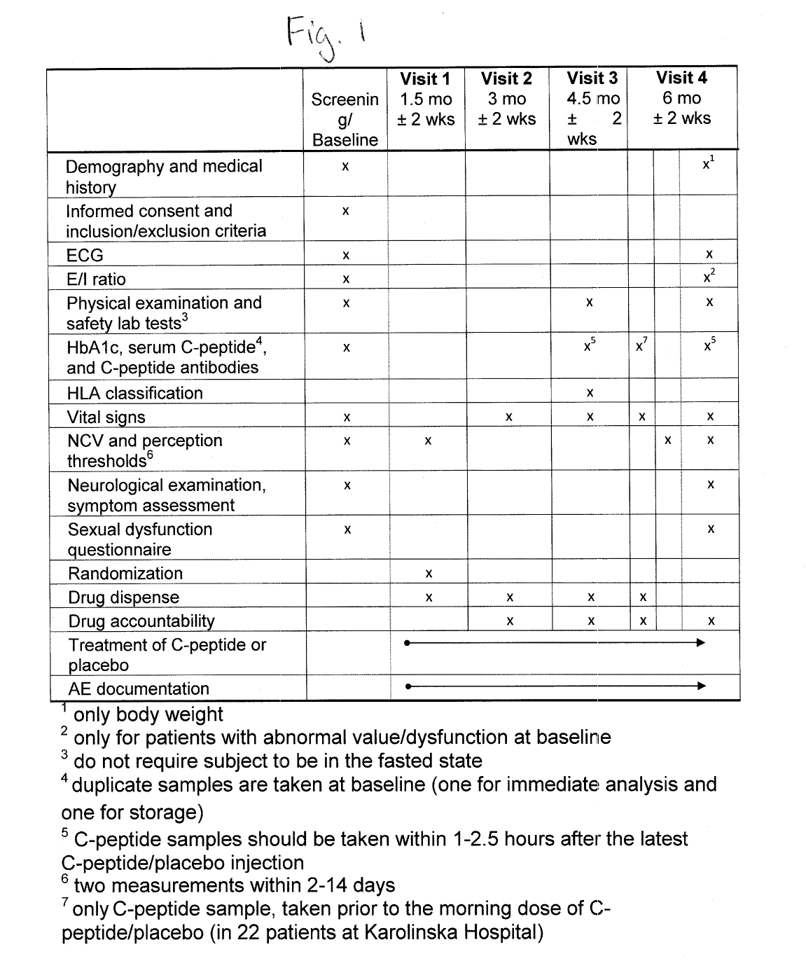

At the final clinical visit (at 6 months from S/B visit), a safety assessment including physical examination, ECG, vital signs, body weight, and sampling for clinical safety laboratory tests were performed. Furthermore, the metabolic control (HbA1c) was assessed, as well as C-peptide antibodies and C-peptide levels in plasma. All efficacy variables including SCV, SNAP, MCV, MDL, CMAP, QST were performed (two examinations within 2-14 days). E/I ratio and neurological status were also assessed. All used and unused clinical trial medication was returned by the patient and drug accountability was performed by study staff. The patient diary was reviewed including recording of concomitant medication, AEs, and drug accountability. FIG. 1 summarizes the study procedures and assessments conducted at each time point.

### A Selection of the Study Population & Inclusion Criteria

Patients fulfilling the following criteria were eligible for participation in this study:
given informed written consent
age between 18-55 years
BMI<30 kg/m²
duration of type 1 diabetes of more than 5 yrs
normal renal function as assessed by serum creatinine below 120 μmol/L
plasma or serum C-peptide<0.15 nmoL/L
HbA1c<12%
patients should have a diabetic distal symmetric neuropathy, according to
the criteria defined at the San Antonio Conference on Diabetic Neuropathy (see below)
patients should have a measurable action potential (SNAP) in both sural nerves
patient should have a SCV lower than 1.5 SD from body height-corrected reference value (defined as mean of both legs).

Criteria for Diabetic Neuropathy

The patients included in the study should have a diabetic distal symmetric neuropathy, according to the criteria defined at the San Antonio Conference on Diabetic Neuropathy (American Diabetes Association et al. 1988), i.e., 2 out of 5 criteria below should be fulfilled. The respective criteria are defined as:

1. Symptoms of neuropathy, here defined as ≧1 point in the “Symptom assessment score”.

2. Findings of neuropathy at clinical examination, here defined as ≧8 points in the “NIA score”.

3. Findings of neurophysiological examination from at least 2 nerves, here defined as <−1.5 SD of reference values (mean of both nerves, and body height-corrected for SCV and MCV).

4. Finding on QST, here defined as >1.5 SD of reference values (mean of both legs) for at least one of the perception threshold variables for vibration, heat, and cold.

5. Pathological autonomic function test, here defined as an abnormal value (<−1.5 SD from reference value).

However, the decision had to be based on pathological findings for criteria 1, 2, or 4. Consequently, the 5^{th} criterion was not included in practice.

Exclusion Criteria

Patients presenting any of the following exclusion criteria were not included in the study:
have been treated with any other investigational drug in the last 3 months before entering the study and have taken part in any other clinical trial including intervention during the duration of the study
have known hypersensitivity against C-peptide, m-cresol, glycerol, or sodium phosphate
have history of chronic alcohol or drug abuse within the last year or present
been pregnant, breast feeding, or a women of childbearing potential not using adequate birth control (IUCD, barrier-method, oral contraceptive, abstinence)
have any medical or psychiatric condition, which could jeopardize or would compromise the patient's ability to participate in this trial or decrease the likelihood of obtaining satisfactory data to achieve the objective of the trial
have neuropathy or signs of nerve dysfunction which may be a consequence of factors other than type 1 diabetes
been transplanted (islet cell, kidney, or pancreas)
have had an amputation or wounds in the lower limbs that prohibit or interfere with the performance of neurological or neurophysiologic examinations
have unstable glucose control in the clinical judgment of the investigator
have concomitant medication such as oncolytic therapy or treatment with, e.g., steroids, tricyclic antidepressive, Ca²⁺-channel blocker, antiepileptic agents, i.e.,
   drugs that may interfere with the peripheral nerve function or measurement thereof.

Removal of Patients from Therapy or Assessments

Patients could withdraw her/his consent at any time without giving reasons and without prejudice to further treatment. A patient could also be withdrawn from the trial at any time for the following reasons at the discretion of the investigator or sponsor in the event that:
The investigator judged it necessary and in the best interest of the patient, in particular in case of a serious adverse event (SAE) or an inter-current disease.
If the patient failed to conform to the requirements of the study.
If the investigator considered the patient to be non-compliant in general and, in the opinion of the investigator, decreases the chance of obtaining satisfactory data to achieve the objective of the trial.
If a female patient became pregnant.
The patient could discontinue on the basis of an abnormal laboratory value. The investigator reviewed all abnormal values. If the abnormal value was considered to be of clinical relevance, the test was repeated and any action taken recorded.
Major protocol violations.
Inaccurate or incomplete data.
Unsafe or unethical practices.
Questionable safety of the investigational drug or as an administrative decision.
If a center did not recruit patients as planned, the sponsor could replace such a center by another center.

Patients who withdraw from the study were asked for their reason(s) for withdrawal and about the presence of any AEs. The reason(s) and date for withdrawal as well as presence of AEs were documented in the case report form (CRF).

Procedures in Case of Emergency

An envelope containing the randomization code for each patient was kept by the investigator at each site. The envelopes were returned to the trial manager un-opened after study termination. The randomization code was to be broken by opening of the sealed envelopes only in case of emergency when it was necessary to know the study medication or the batch number for the proper care of the patient, i.e., if an SAE occurred and the knowledge of the study medication was of importance for care of the patient, or required by the authorities. In case the randomization code was broken, the monitor or the trial manager had to be notified promptly either by phone or by fax, followed by a written report stating the name of the person breaking the code, the patient code, date and time, reason for breaking the code and any therapy instituted. If the code was broken due to an AE, the relationship to the study drug should be given.

B Treatments Administered

The trial medication, C-peptide, and matching placebo were supplied in 2.2 mL vials (Disetronic Pen glass vials) of identical appearance, for S.C. injection. The same treatment regimen was used for all patients, i.e., 4 daily injections concomitantly with injection of the regular insulin dose.

### Identity of Investigational Products

Trial Medication 1

| | |
|---|---|
| Name | Recombinant human C-peptide |
| Dosage form | Vials with 2.2 mL aqueous solution |
| | for subcutaneous injection |
| Strength | 3 mg/mL |
| Batch no. | 8 batches of 3 mg/mL were employed |
| Expiry date | Issued along with proven extended |
| | stability of the product in the |
| | Disetronic pen vials |
| Supplier | Creative Peptides Sweden AB |

Trial Medication 2

| | |
|---|---|
| Name | Recombinant human C-peptide |
| Dosage form | Vials with 2.2 mL aqueous solution for subcutaneous |
| | injection |
| Strength | 9 mg/mL |
| Batch no. | 8 batches of 9 mg/mL were employed |
| Expiry date | Issued along with proven extended stability of the |
| | product in The Disetronic pen vials |
| Supplier | Creative Peptides Sweden AB |

Trial Medication 3

| | |
|---|---|
| Name | Placebo |
| Dosage form | Vials with 2.2 mL aqueous solution for subcutaneous |
| | injection |
| Batch no. | 8 batches of placebo were employed |
| Expiry date | Issued along with proven extended stability of the |
| | product in the Disetronic pen vials |
| Supplier | Creative Peptides Sweden AB |

### Packaging and Labeling

The investigational product was packed and labelled according to ICH GMP guidelines and to local law in such a way as to protect the products from deterioration during transport and storage. The investigational product was supplied in Disetronic Pen glass vials filled with 2.2 mL trial medication.

Storage Instructions

The trial medication was stored refrigerated (2-8° C.). Trial medication was shipped by the Sponsor to a central pharmacy (Karolinska Hospital Pharmacy), from which the investigator ordered medication when required in smaller batches. At the sites the investigators were responsible for safe and proper storage of the investigational drugs. Patients were advised to store the boxes with vials in a refrigerator as soon as possible upon receipt at the investigational site.

Procedures for Delivery and Supply

The investigational drug was shipped directly from the filling facility (Skandeborg, Denmark) to the Pharmacy of Karolinska Hospital, where labelling of boxes was performed. Initially, the investigator was supplied with trial medication for the first patients and for subsequent patients additional supplies were delivered from the hospital pharmacy on request.

Drug Accountability

The investigator was responsible for the maintenance of accurate and complete records showing the receipt and administration of investigational drug supplies. All supplies dispensed from the pharmacy during the study were accounted for throughout the study using the Drug Inventory Log which was handed over to each investigator prior to study start.

Method of Assigning Patients to Treatment Groups

At S/B visit each patient received consecutive run-in numbers starting with site no. +1001 (e.g., 21001 for the first subject at site 2). If eligible, patients were given, in consecutive order, randomization numbers starting with 6001 assigning them to start administration of one of three treatment arms of trial medication. When study medication was ordered from the central pharmacy, the pharmacist assigned an appropriate randomization number for that patient (starting with the lowest “free” study medication number in a consecutive order) and thereafter the medication was shipped to the investigational site. At arrival the ordered medication was recorded in the Drug Inventory Log. The eligible patient was given the lowest available number at the site in a consecutive order. The treatment assignment was performed using the Excel random number generator. The randomization was performed by an independent statistician.

Selection of Doses in the Study

The dose selection is based on data from previous studies, in which C-peptide has been replaced for 3 months (600 nmoL/24 h) and an effect on albumin excretion and sensory nerve conduction velocity has been observed (Johansson et al., 2000; Ekberg et al., 2003). In order to maintain adequate levels of C-peptide for as many hours of the day as possible, the daily dose was divided into four doses, injected subcutaneously using Disetronic Pen 25, and administered in the morning, at lunch, at dinner, and at bedtime. With the low dose (500 nmoL/24 h), given 4 times daily, a physiological mean plasma concentration of ˜1 nmoL/L was expected, in keeping with the bioavailability of approximately 80% of subcutaneously administered C-peptide. To determine if C-peptide at a higher dose has an optimal effect, a 3 times higher dose (1,500 nmoL/24 h) was also administered.

Duration of Treatment and Site of Injection

The duration of treatment was 6 months (±2 weeks). The patients were instructed to administer the trial medication subcutaneously as 4 daily injections. The total dose of 500 nmoL, 1,500 nmoL, or placebo per day was divided into 4 portions (⅕+⅕+⅕+⅖) given in the morning (between 6:00-9:00), at lunch (11:00-14:00), at dinner (16:00-19:00) and in the evening before bed (21:00-24:00), i.e., in most cases at the same time as the patient's regular insulin injections. The patients were instructed to inject the trial medication into the abdominal wall, on the opposite side as the insulin injection.

The very first dose was given at the 2^{nd} S/B visit under supervision (i.e., not necessarily in connection to a regular injection of insulin). The investigator or designee instructed and demonstrated to the patient how to inject the trial medication using Disetronic Pen 25, and offered the opportunity for the patient to gain sufficient practical personal experience. Written instructions regarding storage and handling of trial medication were also provided. Study patients received study medication for ˜8 weeks use at S/B visit, visit 1, visit 2, and visit 3. The content of one vial was expected to last for 2 days treatment. At clinical visits 1-4, the patient was asked to return both used and unused vials to the investigator.

Selection and Timing of Dose for Each Patient

The dose of the investigational drug was not individualized, and all patients administered the same dose in each dose arm. The timing of drug administration could vary between patients, however, for reasons of convenience the trial medication was in most cases administered at the time of the patients' regular insulin administration.

Blinding

Blinding was achieved by filling vials with C-peptide low dose, C-peptide high dose, and C-peptide diluent (placebo) solutions of identical appearance. Filling was performed by Pharma Scan, Skandeborg, Denmark, on behalf of Creative Peptides Sweden AB. All vials were labelled with a unique vial number by the Danish filler. Blinding and labelling of the boxes were performed by the Pharmacy at the Karolinska Hospital according to a computer-generated randomization schedule. Neither the investigators or the staff members, nor the patients were aware of which investigational drug was being administered during the trial.

The randomization list was kept unavailable to all persons involved in the study. The blinding was not revealed until the trial had ended and the data file was cleaned, secured, and unblinding was hence decided upon the clean file meeting. Unblinding, inadvertently or due to necessity (if, e.g., an SAE occurred and it was required by the authorities or necessary for the future treatment of a patient) could result in the patient being discontinued from the study. Whether the patient was to remain in the study or not after unblinding was to be a joint decision of the investigator and the trial manager. Any broken code should be clearly justified and explained by a comment on the CRF. However, no unblinding procedures were required during the study.

Prior and Concomitant Therapy

Patients maintained their prescribed therapy for glycemic control and recorded their insulin injections. All other treatments being taken by the patients on enrollment into the study and all treatments given in addition to the study treatment were regarded as concomitant medication.

Patients were instructed not to take any prescription medications or over-the-counter products other than those agreed upon with the investigator. Concomitant medications not permitted during the study were: Ca²⁺-channel blockers, oncolytic therapy or treatment with, e.g., steroids, tricyclic antidepressive, antiepileptic agents, i.e., drugs that may have an influence on nerve function. Administration of medication for acute reasons (e.g., analgesics or antibiotics) was permitted, provided that, the dose, the frequency, and the reason were recorded by the patient in the diary, and data subsequently transferred to the CRF (trade name and/or generic name, timing, and dosage) and in the patient's medical records by the investigator. Therapy, which in the investigator's opinion became necessary during the course of the study, was not refused to the patient, but if the therapy was part of the exclusion criteria, the patient could be withdrawn from the study.

Treatment Compliance

Compliance with administration of investigational drugs was checked by visual control of returned unused and used vials and by review of patient's own recordings of drug administration in the diary. Furthermore, patients were asked about any handling problems according to predetermined questions in the CRF at visits 1, 2, 3, and 4. Compliance was considered sufficient if >80% of the intended total study dose had been administered. Patients with insufficient compliance were not included in the Per Protocol (PP) analysis of data. After completion of the study (post-data base closure), compliance was also checked by qualitative analysis of C-peptide in blood taken at visits 2 and 4.

Efficacy and Safety Variables

Demographics, underlying disease, related diseases, physical examination, and medical history.

At the S/B visit, demographics including date of birth, gender, ethnicity, body height, body weight, body mass index (BMI), and tobacco use were recorded. In addition, data on primary disease was recoded including duration of diabetes, daily insulin requirement, absence or presence of retinopathy (simplex, proliferative), nephropathy (microalbuminuria, proteinuria), and neuropathy (peripheral sensory, autonomic).

A medical history comprising relevant data on past and ongoing diseases was recorded.

Medications (insulin excluded) administered within 4 weeks prior to the S/B visit and ongoing at the time of study start were also recorded. Addition or removal of any medication was recorded throughout the study period.

Physical examinations were performed at the S/B visit, visit 2, and visit 4. ECG was performed at baseline and at end of study, i.e., visit 4 (at 6 months±2 weeks from the S/B visit).

Efficacy assessments.

Insulin Usage

Changes in insulin dosage and requirements were obtained from CRFs and based on a retrospective analysis of patient records reviewed by the investigators for information about potential changes in patients' insulin requirements.

Nerve Conduction Velocities (NCVs)

NCVs were determined twice at baseline and after 6 months of treatment (the mean of the duplicates were used for evaluation). NCV was performed with a technique that was similar to that used in the daily clinical routine with surface electrodes and digital equipment for stimulation and recording. Sensory nerve conduction velocity (SCV) and sensory nerve action potential (SNAP) amplitude bilaterally in the sural nerves, and motor nerve conduction velocity (MCV), compound muscle action potential (CMAP) and motor nerve distal latency (MDL) were determined bilaterally in the peroneal nerves in duplicate, twice at baseline, and twice after 6 months of treatment. The mean from the two legs and of the duplicates was used for efficacy (unless, in the opinion of the central reader or of the investigator there was a specific reason to disregard a result, e.g., due to poor quality of the assessment or for some other reason, e.g., an acute condition affecting only one leg). The examination was strictly standardized and performed according to instructions in the protocol and performed in a warm room, with the legs warmed with heat pads for at least 10 min prior to the nerve conduction measurements, in order to obtain skin temperatures >32° C.

The digital equipment for stimulation and recording used was a Keypoint®, Dantec Medical A/S, Skovlunde, Denmark or similar, providing a digital output from the recordings of the SNAP and the CMAP. A ground electrode was positioned on the tibial anterior aspect at the middle of the lower leg.

Motor Nerve Assessment

Stimulation. The peroneal nerve was stimulated with constant current pulses (0.10 ms duration, repetition frequency 1 Hz) through a hand-held double electrode probe pressed on the skin over the nerve with the cathode of the stimulator probe in the orthodrome direction and with both electrode disc placed over and parallel to the presumed location of the nerve. The probe had an inter-electrode distance 25 mm center-to-center, and each electrode a diameter of 7 mm. The intensity was increased by monitoring the evoked muscle response in order to establish the supramaximal level. The nerve was stimulated (1) at a fixed distance (80 mm) proximal to the muscle belly of m. extensor digitorum brevis of the foot and (2) at a position below the fibula head on the leg. At each site (1) and (2) a final single stimulus of supramaximal strength was applied and the motor response (see below) was saved for calculation. The distance between the sites (1) and (2) was measured and used in the calculation of velocity.

Motor response. The CMAP was recorded with surface electrodes (metal discs of 7 mm coated with electrode paste) placed over the muscle belly and at a site 50 mm distal to the muscle belly. The signal was recorded with a bandpass filter of 2 Hz to 10 kHz, the amplifier gain 5 mV/division (gain was increased if necessary) and sweep speed of 5 ms/division. The amplitude of CMAP was calculated from the level of the beginning of the response to its negative (upward) peak.

Motor nerve conduction. The MCV was calculated from the ratio of the distance between (1) and (2) over the latency differences between (1) and (2) of the beginning of the muscle response.

Sensory Nerve Assessment

The sural nerve was stimulated with the same stimulator and probe as above with pulses of 0.10 ms duration and a frequency 1 Hz. The site of stimulation was at the wrist on the back of the leg and the recording site was behind the lateral malleolus. Thus antidromic nerve stimulation was used, in order to obtain a larger signal due to the more superficial location of the nerve in the foot as compared to the wrist. After the recording site was established and fixed, the stimulation site was adjusted and measured to be 130 mm measured from the center of the nearest recording and stimulating electrode discs. The stimulation intensity was adjusted to the supramaximal level.

Recording. The recording probe consisted of two metal electrode discs of 7 mm with interelectrode distance 25 mm center-to-center and was placed over and parallel to the presumed location of the sural nerve. The site of the recording electrode was adjusted during repeated nerve stimulation in order to obtain the maximum response and thereafter its position was fixed with a strap around the foot. The amplifier was set to a gain of 20 μV/division, band pass filter 20 Hz to 10 kHz, and sweep speed 2 ms/division. SNAP was averaged during at least 4 (in case of low signals up to 40) stimulations given with a constant and supramaximal intensity, and the response was saved.

Sensory nerve conduction. The SCV was calculated from the ratio distance (130 mm) divided by latency from stimulus onset to the peak of SNAP (SCVp) or the start of SNAP (SCVi). The amplitude of SNAP was the peak value minus the baseline value defined by interpolation between the level at the beginning and the end of the SNAP.

Neurological Examination

The purpose of the neurological examination was to establish the current extent and severity of peripheral polyneuropathy. The examination was made according to a fixed protocol including sensory screening for touch, pin prick, vibration, heat and cold at the levels of the big toe, dorsum of the foot, and the lower leg (−10 cm below the patella), bilaterally. In addition, assessment of joint sense for the big toes and examination of reflexes were included. The observations were compiled into a score, “neuropathy impairment assessment (NIA) score”. In addition, subjects were asked about subjective symptoms using a symptom questionnaire.

Quantitative Sensory Testing (QST)

Thresholds of perception for heat and cold were determined using Marstock technique, twice at baseline, and after 6 months of treatment (the mean of the duplicates are used for evaluation). Taken together the QST measurements provide an evaluation of the degree of functional impairment as well as an indication of the regional distribution of the neurological impairment.

Temperature Thresholds

Temperature thresholds were assessed by the Thermotest® (Somedic AB, Stockholm, Sweden) or similar. A probe with adjustable temperature was applied over the lateral dorsum of the foot and on the anterior aspect of the lower leg, ˜10 cm below the patella. The temperature of the thermode was initially adjusted to 32° C. (baseline). The temperature of the thermode was automatically changed by a rate of 1° C./sec. The patients reported temperature sensations by pressing a button on perception of cold (repeated 5 times) and heat (repeated 5 times), according to the method of limits (Marstock technique). If the results were highly variable, the measurements were repeated. Measurements were done bilaterally and the mean was calculated. In case of a large variability of the threshold this was taken as a sign of abnormal sensation.

Vibration Thresholds

Vibration thresholds were assessed by the Vibrameter® (Somedic AB, Stockholm, Sweden) or similar. The vibrameter was applied on the skin over the first metatarsal (mid foot) and over the tibia about 10 below the knee. The frequency of vibration was 100 Hz and its intensity increased from 0 until the patient reports the feeling of vibration. The procedure was repeated at least three times and the mean threshold value calculated. Measurements were done bilaterally, in triplicate, and the average was calculated

Heart Rate Variability

Autonomic nerve function was tested according to instructions in the protocol by measurement of E/I ratio during deep breathing. The deep breathing test consisted of two 70 sec periods of deep breathing with 3 min rest in between. The subject was told to inhale during 5 sec and exhale during 5 sec, i.e., 7 breathing cycles during each 70 sec period. The ratio between the longest R-R interval during expiration and the shortest R-R interval during inspiration was calculated breath by breath and the mean E/I value of 5 breaths was reported.

Evaluation of E/I ratio was not to be performed in subjects who were on treatment with sympatomimetic agents or β-blockers. A renewed assessment after 6 months of treatment was only performed in those subjects that presented with a pathological E/I ratio at baseline.

Sexual Function

Sexual performance and erectile dysfunction were analysed via the use of a self assessment questionnaire which was based on International Index of Erectile Dysfunction questionnaire developed for clinical practice and studies of Erectile Dysfunction (Rosen R C, et al. Urology; 49: 822-830 (1997)).

The questionnaire included 10 questions, the first four specifically intended for evaluation of ED. Each question had five alternative answers arranged in an ordinal way and coded 1-5, where 1 point indicated severe dysfunction and 5 no dysfunction. In analyses of overall treatment effects data from ten of the questions have been used.

For the purposes here, a combined score of less than 16 for the four ED specific questions was considered evidence of ED. Of the total 78 males who participated in the trial, fifty of these subjects (39 on active treatment and 11 on placebo) completed answers to the first four questions of the SHIM both at baseline and at the end of the study. Their data have been used in the analyses.

In the calculation of changes during the study, score points at baseline were deducted from points at study termination. A positive result indicates improvement and a zero or negative result no change or worsening. The individual results for the ten questions were added to form a variable reflecting change in overall sexual performance. Further, the data has been dichotomized in a way that a positive change has been coded 1 and a worsening or no change coded 0. The sum of dichotomized improvements (range: 0-10) for each subject was also calculated.

Partial data from 72 of the 78 males who participated in the study were available. Ten of the men were not sexually active. In addition, incomplete data only were available for 12 patients. Thus, results for a total of 50 patients could be analysed. Age and duration of diabetes at study start for the subjects with complete data were 45±7 years and 28±10 years, respectively.

Statistics

Non-parametric methods were employed. The Mann-Whitney test was used in comparisons of results from C-peptide and placebo-treated patients. Fisher's exact test was used for analyses of four field tables. In correlation analyses, Spearman's rho was used. The binomial test was used on the dichotomized data for overall testing of improvement between groups. The change in scale points was also calculated and analysed. A two-tailed test yielding a P-value of <0.05 was considered a statistically significant outcome. The standard statistical package (SPSS) for Windows, V15.0 (SPSS Inc, Chicago, Ill., USA) was utilized.

Laboratory Assessments

### Hematology, Clinical Chemistries, and Urinalysis

The following laboratory tests were performed at the S/B visit, visit 2, and at end of study (visit 4):

### Other Tests:

The following laboratory tests were performed at the S/B visit, visit 2, and at end of study (visit 4):

HbA1c (metabolic control) and in women a urine sample for determination of HCG (human chorion gonadotropine)

C-peptide in plasma

C-peptide antibodies

HLA typing (only at visit 2)

Markers for atherosclerosis

The total amount of blood drawn during the study did exceed 100 mL per patient.

### Labelling and Handling of Samples

Laboratory tests for haematology, clinical chemistries, urine analysis, HbA1c, and HCG, as well as C-peptide plasma levels for screening (inclusion criteria) were analyzed at each site's local Department of Clinical Chemistry according to their standard operating procedures.

C-peptide plasma samples, C-peptide antibodies, and markers for atherosclerosis were processed (centrifuged, etc.) and stored frozen until transport to a central lab. HLA typing samples were shipped to a central lab for processing and storage until analysis. All samples there were labelled uniquely, including information about screening number and sample time.

### Safety Measurements

The safety measurements comprised collection of AEs, spontaneously reported by the patient or recorded by the investigator at each clinical visit. Review of laboratory investigations, vital signs, and physical examinations was performed at the S/B visit, visit 2, and at end of study (visit 4). ECG was performed at the S/B visit and at visit 4.

### Adverse Events

An AE was classified as “unexpected event” (i.e., if not specifically listed in the Investigator's Brochure) in terms of nature, severity, or frequency. All AEs, including intercurrent illnesses and increased severity or frequency of sign/symptoms of a concomitant disease was reported and documented as described below. AEs were documented on an “Adverse Event/Serious Adverse Event” page of the CRF and in the patient's medical records. Any AE occurring at any time after the end of the study, considered to be caused by the study medication—and therefore a possible adverse drug reaction—was reported to the sponsor.

The following observations were also to be considered as AEs:

Laboratory value(s) out of reference range and of clinical concern

Laboratory value(s) changed from patient's baseline value and considered of clinical concern

Pre-existing physical findings which worsened compared to baseline and which believed to be clinically significant

Physical findings (including vital sign) observed during the trial and which believed to be clinically significant.

Hypoglycemic events are common events in insulin-treated patients. For practical reasons only severe hypoglycemic events, i.e., if the patient required assistance from another person in order to regain normoglycaemia, were reported as an AE.

The investigators were asked to follow up all unresolved AEs during 30 days after treatment termination or until a stable status was achieved. This follow-up information was collected in the AE form.

### Non-Immediate Reporting of All AEs

All non-serious AEs were recorded on the AE form in the CRF. The AE information was collected on a regular basis during the clinical trial by the trial manager and transferred to the Sponsor.

### Immediate Reporting of SAEs

Regardless of severity all serious adverse events (SAEs) which occurred during the duration of the study were reported within 24 hours by telephone or telefax to the Clinical Project Manager or Drug Safety Officer of the Sponsor and in writing within 5 days. As far as possible, all points on the “Adverse Event/Serious Adverse Event” page of the CRF were covered in the initial telephone report or the completed form. The form was then sent by mail to responsible staff of Creative Peptides. After receipt of the initial report, the responsible staff reviewed the information and contacted the investigator, if necessary, to obtain further information for assessment of the event. When required, a follow-up report including all new information obtained on the serious adverse event was prepared and sent to Clinical Project Manager or Drug Safety Officer of the Sponsor. The report was marked “Follow-up report”. Copies of all SAEs reported during the study were submitted to the authorities.

### Assessment of Laboratory Values

Coagulation, Hematology, Clinical Chemistry

Before starting the study, the investigator supplied the Sponsor with a list of the normal laboratory ranges and units of measurement for the laboratory variables to be determined during the study at the site. All abnormal laboratory (those out of the normal range) values required comments on the CRF, regardless of the clinical significance:

Error—Include improper sample preparation, hemolysis, delayed transit to laboratory, etc.

Not relevant—Abnormality that was not alarming.

Subject condition—Abnormality that was a consequence of the subject's disease, age, etc.

Adverse event—Clinically relevant abnormal value that cannot be explained by the above assessment flags. Adverse Event/Serious Adverse Event CRF was filled in.

Assessment of Adverse Events

The period of observation for AEs extended from the time the patient started on trial medication until end of the study. AEs were divided into the categories “serious” and “non-serious”. This determined the procedure used to report/document the AE. An SAE is any untoward medical occurrence that:

results in death,

is life-threatening,

requires hospitalization or prolongation of existing hospitalization,

results in persistent or significant disability/incapacity,

is a congenital anomaly/birth defect

other “important medical events”.

“Important medical events” that may not result in death, be life-threatening, or require hospitalization may be considered a SAE when, based upon appropriate medical judgment, they may jeopardize the patient and may require medical or surgical intervention to prevent one of the outcomes listed in the definition. Examples of such medical events include allergic bronchospasms requiring intensive treatment in an emergency room or at home, blood dyscrasias, or convulsions that do not result in hospitalization, or the development of drug dependency or drug abuse.

“Life-threatening” means that the patient is at immediate risk of death from the event as it occurred. It does not include an event that, had it occurred in a more severe form, might have caused death. “Requires hospitalization or prolongation of existing hospitalization” should be defined as hospital admission/prolongation required for treatment of the AE. Hospital admission for scheduled elective surgery would not be an SAE. “Disability” means a substantial disruption of a person's ability to conduct normal life functions.

AEs, which do not fall into these categories, are defined as non-serious. It should be noted that a severe AE need not be serious in nature and that a serious AE need not, by definition, be severe.

Regardless of the classification of an AE as serious or non-serious (see above), its severity was assessed as mild, moderate, or severe, according to medical criteria alone:
mild=does not interfere with routine activities
moderate=interferes with routine activities
severe=impossible to perform routine activities

Patient was instructed by the investigator to report the occurrence of any AE. All AEs, regardless of severity, were followed up by the investigator until satisfactory resolution. All patients experiencing AEs—whether considered associated (means associated if reasonable possibility that the event may have been caused by the drug) with the use of the study medication or not—was monitored until symptoms subside and any abnormal laboratory values had returned to baseline, or until there was a satisfactory explanation for the changes observed, or until death, in which case a full pathologist's report was supplied, if possible. All findings were reported on an “Adverse event” page in the CRF and in the patient's medical records.

Withdrawal from the clinical study and therapeutic measures was done at the discretion of the investigator. A full explanation for the discontinuation from the clinical study was made on the appropriate CRF.

The sponsor provided all investigators involved in a clinical investigation with information regarding clinically relevant AEs.

Primary Endpoints

The primary efficacy variable was the nerve conduction velocity in the sural nerve (SCV) and more specifically, the change of conduction velocity from baseline to 6 months (visit 4). The assessment of SCV was determined bilaterally twice at the S/B visit and at visit 4 and the mean of the 4 recordings, respectively, was used for evaluation of efficacy. The measurement of SCV was strictly standardized.

Drug Concentrations Measurements

Blood samples for determination of C-peptide in plasma were drawn at the S/B visit, visit 2, and at end of study (visit 4). The baseline assessments primarily aimed at demonstrating the extent of C-peptide deficiency in the study population.

The sampling of C-peptide in plasma at visits 2 and 4 coincided with the clinical visit and the time between the most recent administration of the trial drug and sampling varied between patients. Thereby, the result of C-peptide concentrations in plasma was less suitable for pharmacokinetic evaluations, but the results contributed to the assessment of treatment compliance.

Data Quality Assurance

A site visit was performed by the trial manager and the monitor prior to the start of the trial, to review the protocol in detail with the investigator and to assure the availability of appropriate study personnel and their ability to properly conduct the study according to Good Clinical Practice (GCP) procedures.

The study site was visited periodically during the study by the monitor to check the clinical facilities and that the investigational team adhered to the study protocol and that the results of the study were recorded accurately in the CRFs. An audit was performed at one site (Uppsala University Hospital) and at the central pharmacy (Karolinska Hospital) during the study. No major violations were found at the audits. During monitoring visits, reported data were reviewed with regard to accuracy and completeness and data were verified against source documents (e.g., patient files, ECG recordings, laboratory notes, etc.). All data reported in the CRF were supported by source documents unless otherwise stated by the source data verification list.

Data Management

The Case Report Forms (CRF) were monitored and edited by the investigator. CRF data was subsequently transferred to a database and accuracy checked by double-entry of data. The data were sent electronically as a Microsoft Excel file to the data manager/statistician, as well as to an additional statistician. The data manger/statistician then transferred the data to a SAS database and performed statistical calculation according to a predetermined statistical analyses plan. The second statistician transferred the data into a SPSS program, performed calculations, and the results of these were confirmed to be identical to the results of the first statistician. All primary and secondary variables in the SAS database were proofread checked against the data in the CRF. After cleaning the file, all data was transferred to a CD-ROM (read-only-memory).

Statistical Analysis of Primary and Secondary Variables

Descriptive statistics were performed on all variables collected. Analyses were performed on an intent-to-treat (ITT) and a per-protocol (PP) basis. The primary efficacy variable was change from baseline to 6 months of treatment for SCV and comparisons of changes between the treatment groups. Non-parametrical statistical tests (Wilcoxon type) were used. Mean, standard deviation, median, and ranges are presented herein. An improvement of the patient's glycemic control (HbA1c) was expected in all treatment groups and this change was evaluated. Exploratory multivariate analyses including possible predictors of the dependent variables was performed on the primary efficacy variable (change baseline to 6 months) using HbA1c as one of the predictors.

Statistical Analysis of Safety Variables and Adverse Events

Descriptive statistics were used for safety variables. For lab parameters summary statistics were presented as change from baseline and between doses. The changes from baseline were calculated and compared with normal ranges. The safety analysis set included all patients who obtained at least one dose of the investigational drug. AEs were coded according to the MedDRA dictionary. The frequencies of AEs are tabulated by body system and included/preferred term.

Determination of Sample Size

The power analyses of this study was based on the experience from the 3 months treatment study on sub-clinical diabetic neuropathy, where SCV was improved by 2.7 m/s in the C-peptide treated group and the common SD for the two groups (active and placebo) was 4.07 m/s. The following estimates were done:

Primary Analysis, Comparison Between Placebo and Active Treatment

A two group t-test with a 0.050 two-sided significance level will have 80% power to detect the difference in change (baseline to 6 months) between a Group 1 (placebo) mean, μ₁, of 0 m/s and a Group 2 (active drug: high+low dose) mean, μ₂, of 2.7 m/s, a difference in means of −2.7 m/s, assuming that the common standard deviation is 4.07 m/s, when the sample sizes in the two groups are 28 and 56, respectively (total sample size 84).

Secondary Analysis, Differences Between Low and High Dose

A two group t-test with a 0.050 two-sided significance level will have 80% power to detect the difference in change (baseline to 6 months) between a Group 1 (low dose) mean, μ₁, of 2.7 m/s and a Group 2 (high dose) mean, μ₂, of 5.0 m/s, a difference in means of −2.3 m/s, assuming that the common standard deviation is 4.07 m/s, when the sample sizes in the two groups are 51 and 51, respectively (total sample size 102).

Secondary Analysis Test of Equivalence of Effects of High and Low Dose

When the sample size in each group is 40, a two group 0.05 one-sided t-test will have 80% power to reject the null hypothesis that the effects of high and low doses are not equivalent (the difference in means, μ_{H}−μ_{L}, is 2.3 m/s or further from zero in the same direction) in favor of the alternative hypothesis that the means of the two groups are equivalent, assuming that the expected difference in means is 0.0 and the common standard deviation is 4.07 m/s.

In accordance with the above calculations the intention was to include 50 evaluable patients per group, i.e., a total of 150 evaluable patients.

II Study Patients

Disposition of Patients

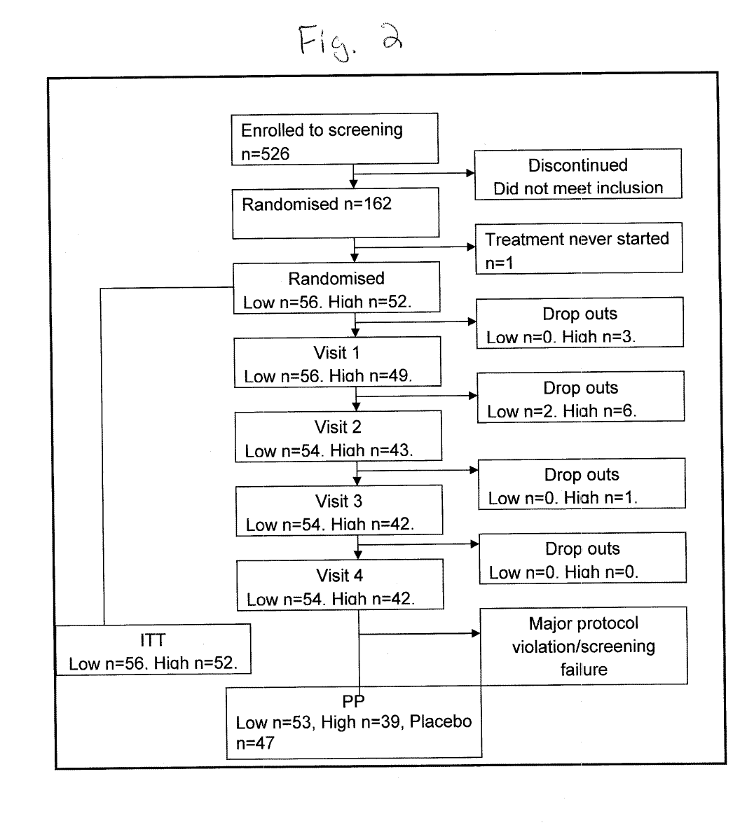

Patients were recruited to the study by advertisements and by screening of hospital records of patients with type 1 diabetes. All patients reporting interest for participation in the study were screened for eligibility. One-hundred-sixty-one (161) patients met the inclusion criteria. All these patients were found eligible and were randomized to the investigational drugs, 56 patients to low-dose C-peptide, 52 patients to high-dose C-peptide, and 53 patients to placebo. Additionally, one patient was randomized but found not to be C-peptide negative and, consequently, did not start the treatment (Table E1, FIG. 2).

Screening Deviations

Among the randomized patients (n=162) the following were included in the study in spite of minor violations to the inclusion/exclusion criteria, Table E1.

TABLE E1
Screening deviations

| Low-dose | | |
|---|---|---|
| C-peptide | High-dose C-peptide | Placebo |
| Reason | Reason | Reason |
| Not applicable | Pregnant at screening/baseline | The baseline C-peptide |
| | visit - not noticed by site staff; | was above acceptance |
| | withdrawn from the study | limit for participation - |
| | immediately when apparent (at | patient never started |
| | visit 1) | the treatment |
| | Suspected alcohol abuse, but | |
| | denied by patient. Confirmed | |
| | when patient had completed the | |
| | study. | |

### Minor Protocol Deviations, Patients Included in the Per Protocol (PP) Analyses

The following patients were included in the PP analysis in spite of minor protocol violations during the study, Table E2.

TABLE E2
Minor protocol deviations

| Low-dose C-peptide | High-dose C-peptide | Placebo |
|---|---|---|
| Reason | Reason | Reason |
| C-peptide sample result at V2 | C-peptide sample result at V2 | C-peptide sample result at V2 |
| viewed by monitor by mistake | viewed by investigator and | viewed by investigator and study |
| | study nurse by mistake (not | nurse by mistake (not seen by |
| | seen by neurologist or | neurologist or |
| | neurophysiologists) | neurophysiologists) |
| Suspected activation of | C-peptide sample result at V2 | Concomitant medication: low |
| multiple sclerosis, but without | viewed by investigator and | dose lithium during study |
| symptoms at S/B and V4 | study nurse by mistake (not | |
| | seen by neurologist or | |
| | neurophysiologists) | |
| Patient has HIV, disease | Vitamin B₁₂ deficiency - oral | |
| status and concomitant | replacement started, disease | |
| medication unlikely to affect | status or concomitant | |
| nerve function | medication unlikely to affect | |
| | nerve function | |
| Patient has rheumatoid | Concomitant medication: low | |
| arthritis, diagnosed 2000, and | dose felodipine started during | |
| due to short duration this is | study | |
| unlikely to affect nerve | | |
| function | | |
| NCV measured at a skin | Concomitant medication: | |
| temperature <32° C. | felodipine ongoing at S/B, | |
| | stopped after 2 months | |
| NCV measured at a skin | | |
| temperature <32° C. | | |

S/B = screening/baseline;
V2 = visit 2;
V4 = visit 4

Other minor protocol violations:
time between visits, was shorter or longer than stated in the protocol (45 patients)
inappropriate use of the study medication; vials used >2 days and/or doses were combined (60 patients)
had medical conditions and/or concomitant medication that potentially might influence nerve function (23 patients)

Minor Protocol Deviations, Patients Excluded from the PP Analyses

For the following protocol deviations, decisions were taken that variables should be excluded from PP analyses, Table E3.

TABLE E3
Minor protocol deviations excluded from the PP analyses

| Minor protocol | | | | |
|---|---|---|---|---|
| deviations excluded | Treatment | | | |
| from the PP | Low-dose | High-dose | | |
| analyses | C-peptide | C-peptide | Placebo | Decision |
| E/I ratio measured | 6 patients | 4 patients | 5 patients | E/I data from these |
| despite patient taking | | | | patients will not be |
| β-blocker or | | | | evaluated |
| sympaticomimetic | | | | |
| drugs | | | | |
| S/B sex | | | 1 patient | Sex Quest data |
| questionnaire filled in | | | | from this patient will |
| after treatment start | | | | be excluded from |
| | | | | the PP analysis |
| Treatment started | 1 patient | | | SB2 data from this |
| prior to S/B2 | | | | patient will be |
| measurement of NCV | | | | excluded in the PP |
| and QST | | | | analyses |
| NIA performed after | | 1 patient | 1 patient | NIA data from |
| treatment stop | | | | these patients will |
| | | | | be excluded from |
| | | | | the PP analysis |
| Treatment stopped | 1 patient | | | V4:2 data from this |
| prior to measurement | | | | patient will be |
| of NCV and QST at | | | | excluded from the |
| V4:2 | | | | PP analyses |

### Major Protocol Deviations

Three patients in the high-dose C-peptide group, one patient in the low-dose C-peptide group, and one patient in the placebo group had major protocol deviations and were excluded from the PP analysis of efficacy, Table E4.

TABLE E4
Major protocol deviations

| Low-dose C-peptide | High-dose C-peptide | Placebo |
|---|---|---|
| Reason | Reason | Reason |
| Unsuccessful compliance | Shortened treatment period (4 | Medical condition, patient had |
| based on review of patient | months) due to summer | hypothyreosis, onset between |
| diary, only ~65% of study | vacation | visits 2 and 3, which could |
| medication was taken | | influence nerve function |
| during the study | | |
| | Suspected alcohol abuse, but | |
| | denied by patient. Confirmed | |
| | later, when completed the | |
| | study. | |
| | Unsuccessful compliance | |
| | based on review of patient | |
| | diary, only ~54% of study | |
| | medication was taken during | |
| | the study | |

### Patient Discontinuations

Fifteen (15) patients withdrew their consent to participate in the study, and two patients were withdrawn from the study, Table E5.

TABLE E5
Patient discontinuations

| Low-dose C-peptide | High-dose C-peptide | Placebo |
|---|---|---|
| Reason | Reason | Reason |
| Depression worsened, pain | Injection needle too rough | Too busy, unwilling to |
| hand/feet, hyperglycemia and | (WC) | continue (WC) |
| worsening of sight problem | | |
| (WC) | | |
| Too big burden of work (WC) | Pregnancy (screening failure) | Increased appetite and |
| | | unstable blood glucose (WC) |
| | Family reasons (WC) | Pain and haematoma at |
| | | injections (WC) |
| | Too many injections (WC) | Failed to keep appointments, |
| | | did not manage the study |
| | | (WC) |
| | Failed to keep appointments | Family problems (WC) |
| | on several occasions (WC) | |
| | Too busy (WC) | |
| | Patient frightened for | |
| | worsening of her retinopathy | |
| | (WC) | |
| | Relapse of rheumatoid | |
| | arthritis (AE) | |
| | Subject in police custody | |
| | (AE) | |
| | Increased incidence of | |
| | hypoglycemic events (WC) | |

WC = patients withdrawn consent;
AE = adverse event

III Efficacy Evaluation

### Data Sets Analyzed

Two data sets were created based on patient evaluability, the Intent-To-Treat (ITT) and Per-Protocol (PP) data sets. The ITT data set comprises all 161 patients who attended the first clinical screening visit (excluded is the one patient who was randomized but never started study medication).

The PP data set is a subset of the ITT data set excluding patients with major protocol deviations as defined in the study protocol or other major protocol violation not foreseen in the study protocol. With respect to the PP data set, the patient evaluability was decided upon before declaring clean file and breaking the treatment code.

The evaluation of the primary and secondary efficacy variables were based on the PP data set whereas the evaluation of safety data was based on the ITT data set. Missing values were handled according to the last-value-carried-forward (ITT LVCF) technique.

In addition, a third data set comprising those patients in the PP data set with a SCVp at screening/baseline better than −2.5 SD, henceforth referred to as “SCVp>−2.5 at SB”.

Demographics and Other Baseline Characteristics

The demographics and key characteristics of the study patients are summarized in Table E6. All treatment groups were well matched with no statistically significant differences between the groups with respect to the demographic variables.

TABLE E6
Demographics and key characteristics of the study population (ITT data set) (n = 161)

| | Low-dose C-peptide | | | High-Dose C-peptide | | | Placebo | | |
|---|---|---|---|---|---|---|---|---|---|
| | | Median | | | Median | | | Median | |
| | Mean ± SD | (min; max) | | Mean ± SD | (min; max) | | Mean ± SD | (min; max) | |
| Age (years) | 44 ± 7.3 | 44 | (29; 55) | 43.5 ± 7.2 | 44.5 | (25; 55) | 42.5 ± 7.6 | 43 | (22; 54) |
| Gender | 20/36 | | | 22/30 | | | 29/24 | | |
| female/male | | | | | | | | | |
| Body weight | 78 ± 12.8 | 76.3 | (54; 110) | 73.8 ± 12.1 | 73 | (51; 102) | 74.3 ± 10.7 | 73 | (59; 105) |
| (kg) | | | | | | | | | |
| Height (cm) | 176.6 ± 10.8 | 177 | (156; 205) | 172.6 ± 8.2 | 172.5 | (148; 190) | 172.4 ± 9.7 | 173 | (148; 200) |
| BMI (kg/m²) | 24.9 ± 2.8 | 24.6 | (19.2; 29.7) | 24.7 ± 3.1 | 24.0 | (17.6; 30.0) | 24.9 ± 2.5 | 24.6 | (20.6; 30.0) |
| Diabetes | 31 ± 8.8 | 31.5 | (6; 48) | 28.2 ± 11.1 | 27 | (11; 51) | 28.7 ± 9.3 | 29 | (10; 48) |
| duration (yrs) | | | | | | | | | |
| Age at | 13.8 ± 8.1 | 12.5 | (3; 45) | 16.1 ± 11 | 13 | (0; 38) | 14.6 ± 8.7 | 13 | (3; 32) |
| diabetes | | | | | | | | | |
| onset (yrs) | | | | | | | | | |
| Insulin dose | 0.64 ± 0.14 | 0.63 | (0.41; 0.98) | 0.69 ± 0.21 | 0.65 | (0.38; 1.51) | 0.63 ± 0.18 | 0.62 | (0.28; 1.14) |
| (IUkg/24 h) | | | | | | | | | |
| HbA1c (%) | 7.57 ± 1.19 | 7.45 | (4.6; 10.2) | 7.60 ± 1.31 | 7.55 | (5.1; 11) | 7.91 ± 1.36 | 7.90 | (4.9; 10.9) |
| C-peptide at | 0.01 ± 0.03 | 0.00 | (0; 0.16) | 0.03 ± 0.04 | 0.00 | (0; 0.20) | 0.01 ± 0.01 | 0.00 | (0; 0.07) |
| screening | | | | | | | | | |
| (nmoL/L) | | | | | | | | | |

Disposition of patients

| | | | |
|---|---|---|---|
| Safety ITT | 56 | 52 | 53 |
| (n) | | | |
| Efficacy PP (n) | 53 | 39 | 47 |

### Characteristics of the Primary Disease

Baseline data on the primary disease are given in Table E7. The diabetes duration was on the order of 30 years in all groups, ranging from 11-51 years in the high-dose C-peptide group and 6-48 years in the low-dose C-peptide group and 10-48 years in the placebo group. The daily insulin requirements, level of metabolic control (HbA1c), and fasting blood glucose level were similar in all three groups.

TABLE E7
Baseline data for the PP population with “SCVp > −2.5 SD at baseline”

| | Low-dose C-peptide | | High-dose C-peptide | | Placebo | |
|---|---|---|---|---|---|---|
| | n = 31 | | n = 18 | | n = 21 | |
| | Mean ± | Median | Mean ± | Median | Mean ± | Median |
| | SD | (min; max) | SD | (min; max) | SD | (min; max) |
| Age (years) | 44 ± 7.6 | 44 | 45 ± 5.8 | 46 | 43 ± 5.9 | 42 |
| | | (33; 55) | | (35; 54) | | (31; 54) |
| Gender female/male | 21/19 | | 11/7 | | 11/10 | |
| Body weight (kg) | 78 ± 14.1 | 78 | 71 ± 12.6 | 72 | 75 ± 12.2 | 73 |
| | | (54; 110) | | (51; 95) | | (60; 105) |
| Height (cm) | 177 ± 11.7 | 178 | 169 ± 9.9 | 168 | 173 ± 8.7 | 174 |
| | | (159; 205) | | (148; 190) | | (163; 200) |
| BMI (kg/m²) | 24.5 ± 2.8 | 23.8 | 24.9 ± 2.9 | 24.6 | 25.0 ± 2.6 | 24.3 |
| | | (20.1; 29.7) | | (20.7, 29.9) | | (21.9; 30.0) |
| Diabetes duration | 31 ± 9.5 | 31 | 32 ± 12.8 | 29 | 27 ± 7.3 | 26 |
| (yrs) | | (9; 48) | | (12; 51) | | (14; 43) |
| Age at diabetes | 14 ± 8.8 | 13 | 13 ± 11.4 | 12 | 16 ± 9.1 | 16 |
| onset (yrs) | | (3; 45) | | (0; 38) | | (3; 32) |
| Insulin dose | 0.62 ± 0.16 | 0.59 | 0.64 ± 0.14 | 0.62 | 0.61 ± 0.13 | 0.62 |
| (IU/kg/24 h) | | (0.41; 0.98) | | (0.38; 0.98) | | (0.36; 0.78) |
| HbA1c (%) | 7.33 ± 1.13 | 7.40 | 7.54 ± 0.99 | 7.50 | 7.60 ± 1.48 | 7.60 |
| | | (4.6; 9.6) | | (6.1; 9.1) | | (4.9; 10.5) |
| C-peptide at | 0.01 ± 0.02 | 0.00 | 0.03 ± 0.03 | 0.01 | 0.01 ± 0.01 | 0.00 |
| screening (nmoL/L) | | (0; 0.10) | | (0; 0.09) | | (0; 0.05) |

The following number of patients administered their insulin by infusion pump, whereas the remaining patients all used S.C. injections:

Low-dose C-peptide group; n=14

High-dose C-peptide group; n=8

Placebo group; n=16

Conditions related to the primary disease of the study patients (peripheral and autonomic neuropathy retinopathy, nephropathy) reported at the S/B visit are presented in Table E8.

TABLE E8
Diabetes complications (peripheral and autonomic
neuropathy retinopathy, nephropathy) known at
baseline (ITT data set, n = 161)

| | Low-dose | High-dose | |
|---|---|---|---|
| | C-peptide | C-peptide | Placebo |
| | n = 56 | n = 52 | n = 53 |
| | N (%) | N (%) | N (%) |
| No neuropathy | 25 (44.6%) | 21 (40.4%) | 27 (50.9%) |
| Peripheral neuropathy | 21 (37.5%) | 24 (46.2%) | 19 (35.8%) |
| Autonomic neuropathy | 2 (3.6%) | 2 (3.8%) | 1 (1.9%) |
| Both peripheral and | 8 (14.3%) | 5 (9.6%) | 6 (11.3%) |
| autonomic neuropathy | | | |
| No retinopathy | 6 (10.7%) | 10 (19.2%) | 8 (15.1%) |
| Simplex retinopathy | 32 (57.1%) | 20 (38.5%) | 19 (35.8%) |
| Proliferative retinopathy | 18 (32.1%) | 22 (42.3%) | 25 (47.2%) |
| No nephropathy | 48 (85.7%) | 45 (86.5%) | 43 (81.1%) |
| Microalbuminuria | 7 (12.5%) | 7 (13.5%) | 7 (13.2%) |
| Proteinuria | 1 (1.8%) | 0 | 2 (3.8%) |

### Medical History

At the start of the study, 53 patients (95%) in the low-dose C-peptide group, 51 patients (98%) in the high-dose C-peptide group and 50 patients (94%) in the placebo group had other ongoing diseases or medical conditions besides the primary and primary-related diseases, Table E9. Numbers in brackets in the table refer to number of patients in each category. Since a single patient may have several different medical conditions the total number of occurrences do not equal the total number of patients in each treatment group.

TABLE E9
Ongoing diseases or medical conditions, except primary disease at study start (ITT
data set). Number in parentheses equals number of patients per diagnosis.

| | Low-dose C-peptide | High-dose C-peptide | Placebo | |
|---|---|---|---|---|
| Organ | n = 56 | n = 52 | n = 53 | |
| system | Specifications | Specifications | Specifications | Total |
| Metabolic- | Hyperlipidemia (7) | Benign thyroid tumor | Cystic changes in | 29 |
| Endocrine | Hypothyreos (3) | (1) | breast (1) | |
| | Severe hypoglycemia | Hyperlipidemia (3) | Hypothyreos (4) | |
| | with unconsciousness | Hyperthyreos (1) | Hyperlipidemia (3) | |
| | (1) | Hypothyreos (5) | | |
| Cardio | Hyperlipidemia (3) | Angina pectoris (1) | Hyperlipidemia (6) | 50 |
| vascular | Hypertension (14) | Heartbeat palpitaion | Hypertension (10) | |
| | Varicose veins (1) | episodes (1) | | |
| | | Hyperlipidemia (1) | | |
| | | Hypertension (13) | | |
| Pulmonary | | Asthma bronchiale (6) | Bronchial constriction | 10 |
| | | | (1) | |
| | | | Unspecific coughing (1) | |
| | | | Asthma bronchiale (2) | |
| Hepato- | Constipation (2) | Celiachi (1) | Chronic bowel | 30 |
| Gastro | Diarrhea (1) | Colon irritable (1) | inflammation (1) | |
| intestinal | Gastritis (3) | Diffuse gastric and | Gastritis/Duodenitis (3) | |
| | Lactose intolerance (1) | bowel symptom (1) | Gastropares/gastrointe | |
| | Primary biliary cirrhosis | Gastritis (4) | stinal pain/constipation | |
| | (1) | Gluten and lactose | (5) | |
| | | intolerance (1) | Unspecific dyspepsia | |
| | | Small tumor below | (1) | |
| | | umbilicus (1) | | |
| | | Nausea, vomiting (1) | | |
| | | Ulcerative colitis (1) | | |
| | | Diarrhea intermittent | | |
| | | (1) | | |
| Renal/ | Genital Mucosis (1) | Erectile dysfunction (1) | Albuminuria (1) | 13 |
| Genito | Impotence (2) | Myoma uteri (1) | Erectile dysfunction (1) | |
| Urological | Microalbuminuria (1) | | Endometriosis (1) | |
| | Recurrent urinary tract | | Menorraghia (1) | |
| | infection (1) | | | |
| | Ovarian cyst (1) | | | |
| | Incontinence problem | | | |
| | (1) | | | |
| Haematological/ | HIV (1) | Anaemia or B₁₂ | Mild chronic lymphatic | 7 |
| Lymphatic | | deficiency (3) | leukemia (1) | |
| | | Thrombocytosis (1) | PAI-1 deficiency (1) | |
| Dermatological | Acne (1) | Dry skin (2) | Acne (2) | 32 |
| | Dermatomycosis (1) | Eczema (1) | Carbunculosis (1) | |
| | Dura molle (1) | Granuloma anulare (1) | Diabetic dermopathy | |
| | Eczema (7) | Lichea planus (1) | (1) | |
| | Granuloma annuale (2) | Psoriasis (2) | Herpes Infection (2) | |
| | Psoriasis (1) | Vitiligo (2) | Eczema (2) | |
| | Vitiligo (1) | | Herpes zoster (1) | |
| Allergies | Allergic rhinitis (2) | Allergic asthma (1) | Allergy (cats, dogs, | 48 |
| | Allergy (Birch, mold, | Allergic rhinitis (4) | penicillin, foods, pollen) | |
| | furs, nickel, cat, dogs, | Allergy (cats, dogs, | (13) | |
| | penicillin, pollen) (11) | penicillin, pollen) (10) | Rhinoconjunctivitis (2) | |
| | Rhinoconjunctivitis (2) | Stress urticaria (1) | Anxiety, insomnia (1) | |
| | | Urticaria against food | | |
| | | (1) | | |
| Musculo | Carpal tunnel | Arthritis (2) | Bursitis left knee (1) | 87 |
| Skeletal | syndrome (4) | Carpal tunnel | Shoulder pain (2) | |
| | Fibromyalgia (1) | syndrome (1) | Dig I left nail crushed | |
| | Frozen shoulder (4) | Frozen shoulder (3) | (1) | |
| | Head/back/shoulder/ | Neck and shoulder | Left thumb sprained (1) | |
| | neck/leg and/or muscle | stiffness/pain (4) | Carpal tunnel | |
| | pain (4) | Omarthritis (1) | syndrome (10) | |
| | Hip-joint arthritis (1) | Osteopathi (1) | Whiplash (1) | |
| | Arthritis dig I (1) | Osteoporosis (1) | Costal and clavicular | |
| | Omarthritis (1) | Whiplash (1) | fracture (1) | |
| | Headache now and | | Fibromyalgia (1) | |
| | then (1) | | Frozen shoulder (2) | |
| | Rheumatoid arthritis (1) | | Knee injury - arthrosis | |
| | Spontaneous | | (2) | |
| | metatarsal fractures (1) | | Inguinal hernia (1) | |
| | Shoulder/hip stiffness | | Omarthritis (1) | |
| | (2) | | Pain | |
| | Trigger op (1) | | knee/shoulder/neck (3) | |
| | Duputrytren's | | Knee injury (1) | |
| | contracture (1) | | Shoulder/hip/back | |
| | Atralgi, general all | | stiffness (4) | |
| | joints (1) | | Shoulder tendinitis (3) | |
| | Arthritis shoulder (1) | | Finger tendoclevage - | |
| | Whiplash (1) | | multiple (1) | |
| | Artrophati (Arthros) | | Fractures; os | |
| | hands (1) | | naviculare/tibia/fibula | |
| | Osteochondrit op.knee | | (2) | |
| | past (1) | | Osteoporosis (1) | |
| | Epicondylitis elbow (1) | | Duputrytrens | |
| | Heel spur (1) | | contracture (1) | |
| | Head trauma with | | Neckproblem from | |
| | epidural bleeding (not | | arachnoidal cystic | |
| | operated) (1) | | changes (1) | |
| Neurological | Headache/Migraine (8) | Erectile dysfunction (1) | Erectile dysfunction (1) | 28 |
| | Mild numbness in hand | Headache/Migraine | Headache (2) | |
| | (1) | now and then (4) | Mild burning felling in | |
| | Paraesthesias legs (1) | Paraesthesias hand (1) | feet (1) | |
| | Autonomic neuropathy | Vertebra compression | Mild sensation feeling | |
| | (1) | C5-C6 (1) | toe (1) | |
| | Unspecific symptoms | Whiplash (1) | Tension headache (1) | |
| | from feet (1) | Facialis paresis (1) | Vertigo-unclear (1) | |
| | | | Meniere (1) | |
| Sense | Blindness (2) | Blind right eye (1) | Cataract (2) | 24 |
| Organs | Decreased vision (1) | Glaucoma (2) | Impaired hearing (2) | |
| | Reduced hearing (2) | Reduced/Hearing loss | Impaired vision (1) | |
| | Cataract (1) | (3) | Mb Meniere (1) | |
| | | Recurrent vax plug (1) | Enucleated eye dex (1) | |
| | | Tinnitus (1) | Vitreus and retinal eye | |
| | | Vasomotoric rhinitis (1) | op (1) | |
| | | Dystrophia lateral | | |
| | | cornea bilat. (1) | | |
| Other | Lipoma (1) | Dyslexia (1) | Adenoma breast (1) | 8 |
| | Sjogrens syndrome (1) | Episodes of dizziness | Insomnia (1) | |
| | | and tachycardia (1) | Snoring - sleep apnea | |
| | | Weight increase since | (1) | |
| | | years (1) | | |
| Total | 124 | 112 | 130 | 366 |

### Concurrent Medication

The medications taken at study start and during the study are shown in Table E10. Figures in the table refer to number of patients using each category of drug. Since a single patient may have used several medications the total number of occurrences do not equal the total number of patients in each treatment group. Three patients in the low-dose C-peptide group, six patients in the high-dose C-peptide group and six patients in the placebo group had not taken any medication within 4 weeks prior to study start nor had they any ongoing medication during the study.

TABLE E10
Medications taken within 4 weeks of study start and medications taken
during the study (ITT data set). The Anatomical Therapeutic Chemical
(ATC) Classification System is used for the classification of the drugs.

| | | Low- | High- | |
|---|---|---|---|---|
| | | dose | dose | |
| ATC Classification | | C- | C- | |
| System, | ATC Classification System, | peptide | peptide | Placebo |
| Anatomical groups | Therapeutic main groups | n = 56 | n = 52 | n = 53 |
| A. | Drugs for acid-related disorders | 7 | 3 | 10 |
| Alimentary tract and | Drugs for functional gastrointestinal | 1 | 1 | 1 |
| metabolism | disorder | | | |
| | Bile and liver therapy | 1 | | |
| | Laxatives | 1 | 1 | 3 |
| | Antidiarrheals intestinal anti- | 2 | 4 | 3 |
| | inflammatory/anti-infective agents | | | |
| | Antiobesity preparation | | 1 | |
| | Drugs used in diabetes | 2 | | |
| | Vitamins | 5 | 1 | |
| | Mineral supplements | 1 | 3 | 1 |
| B. | Antithrombotic agents | 5 | 12 | 10 |
| Blood and blood | Antihemorrhagics | | 1 | 1 |
| forming organs | Antianemic preparations | | 7 | 2 |
| | Parenteral carbohydrate solution | 1 | | 3 |
| C. | Vasodilators used in cardiac diseases | | 2 | |
| Cardiovascular | Diuretics | 11 | | 5 |
| system | Beta-blocking agents | 7 | 5 | 6 |
| | Calcium channel blockers | 2 | 2 | |
| | Agents acting on renin-angiotensin | 19 | 16 | 16 |
| | system | | | |
| | Serum lipid-reducing agents | 17 | 12 | 13 |
| D. | Antifungals for dermatological use | 1 | | 1 |
| Dermatologicals | Dermatological emollients and | 1 | 1 | 1 |
| | protectives | | | |
| | Antipsoriatics | | 2 | |
| | Antibiotics and chemotherapeutics for | 3 | 1 | |
| | dermatological use | | | |
| | Corticosteroids, dermatological | 1 | 5 | 1 |
| | preparations | | | |
| | Anti-acne preparations | 1 | | |
| | Other dermatological preparation | | 1 | |
| G. | Gynecological anti-infectives and | 1 | | 1 |
| Genito-urinary | antiseptics | | | |
| system and sex | Sex hormones and modulators of the | 3 | 6 | 7 |
| hormones | genital systems | | | |
| | Drugs used in erectile dysfunction | 6 | 3 | 3 |
| H. | Corticosteroids for systemic use | 1 | 2 | 3 |
| Systemic hormonal | Thyroid hormones | 3 | 9 | 8 |
| preparations, | Glycogenolytic hormones | 3 | 1 | 1 |
| excluding sex | | | | |
| hormones and | | | | |
| insulins | | | | |
| J. | Antibacterials for systemic use | 16 | 18 | 17 |
| Anti-infectives for | Antimycotics for systemic use | 2 | | |
| systemic use | Antivirals for systemic use | 5 | 1 | |
| | Vaccines | 1 | | 3 |
| L. | Hormone antagonists and related | 2 | | |
| Antineoplastic and | agents | | | |
| immunomodulating | | | | |
| agents | | | | |
| M. | Anti-inflammatory and antirheumatic | 42 | 23 | 51 |
| Musculo-skeletal | products, non-steroids | | | |
| system | Topical products for joint and muscular | | 1 | |
| | pain | | | |
| | Drugs affecting bone structure and | | | 1 |
| | mineralization | | | |
| N. | Anesthetics, local | 1 | 2 | |
| Nervous system | Analgesics, opioids | 8 | 7 | 6 |
| | Other analgesic and antipyretics | 49 | 23 | 51 |
| | Antimigraine preparations | 4 | 1 | |
| | Antipsychotics | | 3 | 2 |
| | Anxiolytics | 3 | 4 | 1 |
| | Hypnotics and sedatives | 3 | 3 | 8 |
| | Antidepressants | 12 | 9 | 5 |
| | Psychostimulants, agents used for | 1 | 1 | |
| | ADHD and nootropics | | | |
| | Parasympathomimetics | 1 | | |
| | Drugs used in nicotine dependence | 1 | | |
| | General anesthetics | 2 | 1 | 2 |
| P. | Agents against amoebiasis and other | | 1 | |
| Antiparasitic | protozoal diseases | | | |
| products, | Antinematodal agents | | 1 | |
| insecticides and | | | | |
| repellents | | | | |
| R. | Decongestants and other nasal | 1 | 3 | 6 |
| Respiratory system | preparations for topical use | | | |
| | Nasal decongestants for systemic use | 4 | 3 | 1 |
| | Adrenergics, inhalants | 2 | 8 | 4 |
| | Other drugs for obstructive airway | 1 | 3 | 2 |
| | diseases, inhalants | | | |
| | Adrenergics for systemic use | 1 | 1 | |
| | Other systemic drugs for obstructive | 1 | | |
| | airway diseases | | | |
| | Cough and cold preparations | 2 | 5 | 2 |
| | Antihistamines for systemic use | 12 | 8 | 8 |
| S. | Ophthalomolgical | 1 | | |
| Sensory organs | Ophthalomolgical anti-infectives | 4 | 2 | 2 |
| | Ophthalomolgical anti-glaucoma | 1 | 3 | |
| | preparations and miotics | | | |
| | Ophthalmological mydriatics and | 2 | | |
| | cycloplegics | | | |
| | Ophthalomolgical decongestants and | | 1 | 3 |
| | antiallergics | | | |
| | Ophthalomolgical corticosteroids and | 2 | 1 | |
| | anti-infectives in combination | | | |

### Neurophysiological Assessments

Baseline neurophysiological characteristics of the patients are presented in Tables E11 and E12. Nerve conduction velocity in the sural and peroneal nerves was assessed with good reproducibility (coefficient of variation 2-3%). The reproducibility of the compound action potentials had considerably higher variability as known from previous studies.

Sensory nerve conduction velocity in the sural nerves (SCVp) was reduced in the patients at baseline (−2.62 SD as compared to a healthy population). The peak velocity (SCVp) amounted on average to 35.4 m/s and the SCVi to 44.3 m/s (−3.25 SD from normal). Also motor nerve conduction velocity in the peroneal nerves was reduced, mean 40.1 m/s, corresponding to −2.95 SD.

TABLE E11
Baseline data on neurophysiological assessments (ITT data set, n = 161)

| | Low-dose C-peptide | | High-dose C-peptide | | Placebo | |
|---|---|---|---|---|---|---|
| | n = 56 | | n = 52 | | n = 53 | |
| | | Median | | Median | | Median |
| | Mean ± SD | (min; max) | Mean ± SD | (min; max) | Mean ± SD | (min; max) |
| SCVp (m/s) | 35.29 ± 3.42 | 35.76 | 35.78 ± 3.43 | 35.75 | 35.29 ± 3.76 | 36.13 |
| | | (26.68; 43.03) | | (25.18; 42.60) | | (25.40; 41.63) |
| SCVp (SDS) | −2.49 ± 0.95 | −2.30 | −2.59 ± 0.93 | −2.42 | −2.78 ± 1.19 | −2.56 |
| | | (−5.46; −0.83) | | (−5.74; −1.24) | | (−6.92; −1.07) |
| SCVi (m/s) | 44.35 ± 4.52 | 44.89 | 44.65 ± 4.57 | 44.84 | 43.91 ± 5.13 | 43.78 |
| | | (31.75; 56.00) | | (34.90; 54.23) | | (31.53; 52.70) |
| SCVi (SDS) | −3.04 ± 0.97 | −2.95 | −3.26 ± 0.99 | −3.05 | −3.46 ± 1.26 | −3.33 |
| | | (−5.98; −1.34) | | (−5.59; −1.21) | | (−7.50; −1.32) |
| SNAP (μV) | 4.60 ± 3.32 | 3.66 | 5.14 ± 3.65 | 3.85 | 4.33 ± 3.21 | 3.35 |
| | | (0.88; 16.76) | | (0.98; 18.50) | | (0.94; 14.00) |
| MCV (m/s) | 39.29 ± 5.51 | 39.61 | 40.71 ± 3.61 | 39.70 | 40.35 ± 4.42 | 41.05 |
| | | (20.68; 47.65) | | (34.28; 51.13) | | (25.33; 49.58) |
| CMAP (mV) | 3.27 ± 2.33 | 2.83 | 3.51 ± 2.07 | 3.01 | 3.12 ± 2.01 | 3.08 |
| | | (0.13; 12.88) | | (0.33; 9.18) | | (0.15; 8.15) |
| MDL (ms) | 4.59 ± 0.86 | 4.40 | 4.37 ± 0.51 | 4.34 | 4.56 ± 0.86 | 4.45 |
| | | (3.43; 8.33) | | (3.38; 5.68) | | (3.25; 7.90) |

TABLE E12
Baseline data for the PP population with “SCVp > −2.5 SD at baseline”

| | Low-dose C-peptide | | High-dose C-peptide | | Placebo | |
|---|---|---|---|---|---|---|
| | n = 31 | | n = 18 | | n = 21 | |
| | | Median | | Median | | Median |
| | Mean ± SD | (min; max) | Mean ± SD | (min; max) | Mean ± SD | (min; max) |
| SCVp (m/s) | 37.07 ± 2.36 | 37.15 | 38.64 ± 2.41 | 38.14 | 38.01 ± 1.85 | 38.20 |
| | | (31.48; 43.03) | | (34.00; 42.60) | | (33.58; 41.63) |
| SCVp (SDS) | −1.83 ± 0.43 | −1.87 | −1.87 ± 0.35 | −1.92 | −1.78 ± 0.35 | −1.75 |
| | | (−2.50; −0.83) | | (−2.48; −1.24) | | (−2.32; −1.21) |
| SCVi (m/s) | 46.40 ± 3.60 | 46.43 | 48.26 ± 3.55 | 48.03 | 47.50 ± 2.92 | 47.25 |
| | | (39.03; 56.00) | | (41.80; 54.23) | | (42.30 52.70) |
| SCVi (SDS) | −2.46 ± 0.61 | −2.51 | −2.59 ± 0.58 | −2452 | −2.46 ± 0.65 | −2.34 |
| | | (−3.69; −1.34) | | (−3.67; −1.21) | | (−3.73; −1.32) |
| MCV (m/s) | 41.46 ± 3.76 | 41.65 | 43.01 ± 3.83 | 42.55 | 41.85 ± 3.18 | 42.35 |
| | | (33.65; 47.65) | | (37.00; 51.13) | | (33.35; 46.48 |
| MCV (SDS) | −2.28 ± 10.15 | −2.48 | −2.32 ± 0.98 | −2.38 | −2.41 ± 0.74 | −2.34 |
| | | (−4.77; −0.34) | | (−4.23; −0.43) | | (−3.60; −0.89) |

### Quantitative Sensory Assessments

Quantitative sensory testing revealed elevated thresholds at baseline, especially to vibration and cold stimulation, which were more pronounced in the feet than in the lower legs, Table E13. The reproducibility of the perception thresholds assessments were significantly less than for conduction velocity (coefficient of variation for vibration 21%, heat 14%, and cold 21%).

TABLE E13
Baseline data on quantitative sensory function assessments (ITT data set, n = 161)

| | Low-dose C-peptide | | High-dose C-peptide | | Placebo | |
|---|---|---|---|---|---|---|
| | n = 56 | | n = 52 | | n = 53 | |
| | Mean ± | Median | Mean ± | Median | Mean ± | Median |
| | SD | (min; max) | SD | (min; max) | SD | (min; max) |
| Cold threshold foot | 3.04 ± 2.11 | 2.82 | 3.34 ± 2.13 | 3.28 | 3.71 ± 2.11 | 3.96 |
| (SDS) | | (−2.32; 7.37) | | (−0.59; 8.01) | | (−0.96; 7.92) |
| Cold threshold lower | 1.98 ± 1.62 | 1.75 | 2.47 ± 1.99 | 2.43 | 2.50 ± 2.00 | 2.13 |
| leg (SDS) | | (−1.18; 5.65) | | (−1.38; 8.28) | | (−1.14; 8.64) |
| Heat threshold foot | 1.13 ± 0.82 | 1.19 | 1.20 ± 1.03 | 1.24 | 1.40 ± 0.97 | 1.26 |
| (SDS) | | (−0.75; 3.06) | | (−1.68; 3.37) | | (−0.58; 3.61) |
| Heat threshold lower | 0.66 ± 1.06 | 0.75 | 0.93 ± 1.31 | 1.16 | 0.85 ± 1.12 | 0.93 |
| leg (SDS) | | (−3.14; 2.93) | | (−2.79; 3.26) | | (−1.28; 3.38) |
| Vibration threshold | 1.73 ± 1.37 | 1.77 | 1.96 ± 1.48 | 1.75 | 2.06 ± 1.58 | 1.76 |
| foot (SDS) | | (−0.85; 4.40) | | (−0.81; 7.78) | | (−0.79; 6.02) |
| Vibration threshold | 0.49 ± 0.92 | 0.44 | 0.77 ± 1.27 | 0.47 | 0.72 ± 1.13 | 0.51 |
| lower leg (SDS) | | (−1.33; 2.83) | | (−0.98; 6.81) | | (−1.37; 4.37) |

SDS = standard deviation score

### Neurological Impairment Assessments and Symptoms

Pathological neurological findings (NIA>7 points) were present in 86% of the patients at baseline when assessed by the neurological examination. The average NIA score was 17.1 points, Table E14. The reproducibility for the neurological assessment was >25% (coefficient of variation).

TABLE E14
Baseline data on neurological assessment (ITT data set, n = 161)

| Low-dose C-peptide | | High-dose C-peptide | | Placebo | |
|---|---|---|---|---|---|
| n = 56 | | n = 52 | | n = 53 | |
| Mean ± | Median | Mean ± | Median | Mean ± | Median |
| SD | (min; max) | SD | (min; max) | SD | (min; max) |

Neurological assessments

| | | | | | | |
|---|---|---|---|---|---|---|
| Total NIA score | 17.20 ± 9.65 | 16.0 | 16.92 ± 9.45 | 16.50 | 17.15 ± 11.44 | 16.0 |
| | | (0.0; 39.0) | | (0.0; 46.0) | | (0.0; 56.57) |
| Great toe score | 7.23 ± 4.77 | 7.0 | 8.12 ± 4.23 | 8.00 | 7.14 ± 4.40 | 6.0 |
| | | (0.0; 19.0) | | (0.0; 18.0) | | (0.0; 18.0) |
| Foot score | 4.59 ± 3.60 | 4.0 | 4.12 ± 3.41 | 4.00 | 4.13 ± 3.70 | 4.0 |
| | | (0.0; 14.0) | | (−0.0; 16.0) | | (0.0; 16.67) |
| Lower leg score | 1.64 ± 2.03 | 1.0 | 1.65 ± 2.51 | 0.0 | 2.23 ± 3.02 | 0.0 |
| | | (0.0; 8.0) | | (0.0; 12.0) | | (0.0; 14.0) |

Symptom assessments

| | | | | | | |
|---|---|---|---|---|---|---|
| Total symptom score | 0.59 ± 1.06 | 0.00 | 0.75 ± 1.38 | 0.00 | 0.43 ± 0.69 | 0.00 |
| | | (0; 4) | | (0; 6) | | (0; 3) |

Thirty five (35) percent of the randomized patients reported subjective symptoms from the lower limbs at baseline. Seven (7) of the patients presented with symptoms in their upper limbs.

### ECG and Vital Signs

Baseline data on ECG recordings and vital signs are shown in Table E15. Sixteen (16) percent of the patients presented with an abnormal ECG at baseline. The following abnormalities were reported (number of patients in parentheses)—none was of serious clinical significance in the opinion of the investigator:

sinus tachycardia (2)

right electrical axis (2)

minor intraventricular conduction defect (1)

QRS deviation as after inferior myocardial infarction (1)

bundle block (6)

prolonged QTc interval or borderline (4)

slight ST-T changes (3)

TABLE E15
Baseline ECG and vital signs (ITT data set)

| | Low-dose C-peptide | | High-dose C-peptide | | Placebo | |
|---|---|---|---|---|---|---|
| | n = 56 | | n = 52 | | n = 53 | |
| | | Median | | Median | | Median |
| Variable | Mean ± SD | (min; max) | Mean ± SD | (min; max) | Mean ± SD | (min; max) |
| Heart rate | 71.5 ± 11.0 | 72 | 74.3 ± 11.7 | 74 | 73.7 ± 10.2 | 72 |
| (beats/min) | | (51; 104) | | (47; 102) | | (54; 103) |
| QTc-interval | 401 ± 17 | 399 | 404 ± 22 | 400 | 406 ± 21 | 403 |
| (ms) | | (368; 445) | | (350; 460) | | (367; 461) |
| Bazett | | | | | | |
| QTc-interval | 390 ± 16 | 389 | 389 ± 18 | 384 | 393 ± 18 | 391 |
| (ms) | | (358; 432) | | (350; 442) | | (358; 433) |
| Fredericia | | | | | | |
| BPs (mmHg) | 131 ± 15.7 | 130 | 132.7 ± 15.1 | 132 | 133.3 ± 13.9 | 134 |
| | | (102; 180) | | (98; 180) | | (105; 160) |
| BPd (mmHg) | 77 ± 10.1 | 79.5 | 76.1 ± 9.9 | 78.5 | 76.9 ± 7.0 | 78 |
| | | (52; 111) | | (54; 95) | | (60; 90) |

BPs = systolic blood pressure;
BPd = diastolic blood pressure

Clinical Assessments after 3 Months (Visit 2)

Glycemic control (HbA1c) was slightly decreased in all three treatment groups by on average 0.2%.

Clinical Assessments after 6 Months (Visit 4)

Metabolic Control (HbA1c)

The change of metabolic control, as reflected by reduced HbA1c, is presented in Table E16. There was a statistical significant reduction in the low-dose C-peptide group, but the reduction was even greater in the placebo group. Statistical correlation analyses did not show any correlation between the change in HbA1c with the change in SCV after 6 months of treatment.

TABLE E16
Change in HbA1c at 6 months (visit 4) (ITT data set)

| | Low-dose | | High-dose | | | | | |
|---|---|---|---|---|---|---|---|---|
| | C-peptide | | C-peptide | | Placebo | | p < | p < |
| | n = 55 | | n = 43 | | n = 49 | | C- | Low- |
| | Mean ± | | Mean ± | | Mean ± | | peptide | vs. |
| | SD | | SD | | SD | | groups | high- |
| | [p < within | Median | [p < within | Median | [p < within | Median | vs. | dose |
| | group] | (min-max) | group] | (min-max) | group] | (min-max) | placebo | groups |
| HbA1c | −0.21 ± 0.70 | −0.20 | −0.03 ± 0.77 | −0.10 | −0.42 ± 0.72 | −0.30 | ns | ns |
| | [0.013] | (−2.20; 1.90) | [ns] | (−2.20; 1.50) | [0.001] | (−2.30; 0.90) | | |

ns = not significant

Erectile Dysfunction

Baseline Measurements: The average scores for each of the questions for C-peptide and placebo-treated patients are reported in Table E17. The questions focused on erectile function, but also included the patients' evaluation of intercourse satisfaction, sexual desire, ejaculation and relationship to partner. ED, defined as a score of less than 16 points for the first four questions, was present in 44% of patients (n=50) and in 51% of the patients that were to be treated with C-peptide. The summed score for the four questions tended to be correlated to the heart rate variability during breathing (P<0.05), an indicator of autonomic nerve function. In addition, the score was related to the toe and leg score reflecting clinically observable neurological abnormalities (P<0.03-0.05).

TABLE E17
Average score points at baseline and at end of study for the ten questions and for the four ED questions

| | | | | C-peptide treatment, | | | |
|---|---|---|---|---|---|---|---|
| | Placebo, n = 11 | | | n = 39 | | | ΔP vs. |
| | | Study | | | Study | | ΔCP |
| | Baseline | end | Difference | Baseline | end | Difference | P< |
| Erection | 3.5 ± 1.21 | 3.5 ± 0.93 | 0 ± 0.63 | 3.3 ± 1.08 | 3.5 ± 1.02 | 0.2 ± 0.8 | NS |
| confidence | | | | | | | |
| Penetration | 4.4 ± 1.21 | 3.8 ± 1.25 | −0.5 ± 0.93 | 3.9 ± 1.28 | 3.9 ± 1.14 | 0.03 ± 0.58 | 0.043 |
| Erection | 4.3 ± 1.42 | 4.1 ± 1.45 | −0.2 ± 0.75 | 3.8 ± 1.35 | 3.9 ± 1.24 | 0.03 ± 0.67 | NS |
| maintenance | | | | | | | |
| Erection duration | 4.3 ± 1.27 | 3.9 ± 1.22 | −0.4 ± 0.5 | 3.8 ± 1.27 | 4 ± 1.14 | 0.2 ± 0.54 | 0.005 |
| Satisfaction | 4.2 ± 0.98 | 4.2 ± 1.17 | 0 ± 1.26 | 3.9 ± 1.05 | 4.1 ± 0.89 | 0.2 ± 0.84 | NS |
| Sexual desire | 3.9 ± 0.83 | 3.7 ± 0.9 | −0.2 ± 0.6 | 3.5 ± 0.79 | 3.6 ± 0.78 | 0.1 ± 0.85 | NS |
| Premature | 1.3 ± 0.47 | 1.3 ± 0.47 | 0 ± 0.45 | 1.9 ± 1.03 | 1.7 ± 0.9 | −0.2 ± 1.2 | NS |
| ejaculation | | | | | | | |
| Ejaculation failure | 1.1 ± 0.32 | 1.4 ± 0.73 | 0.3 ± 0.46 | 1.5 ± 0.73 | 1.5 ± 0.8 | 0.1 ± 0.63 | NS |
| Relation partner | 3.5 ± 1.21 | 3.5 ± 1.21 | −0.1 ± 0.3 | 3.3 ± 1.12 | 3.4 ± 0.77 | 0.1 ± 0.92 | NS |
| Partner's view | 3.5 ± 1.21 | 3.1 ± 1.22 | −0.4 ± 0.67 | 3.2 ± 1.17 | 3.3 ± 0.82 | 0.03 ± 0.95 | NS |
| All 10 questions | 33.7 ± 7.71 | 32.2 ± 8.07 | −1.5 ± 3.8 | 32.2 ± 5.58 | 32.8 ± 5.44 | 0.7 ± 3.21 | 0.066 |
| Four first ED | 16.4 ± 4.78 | 15.3 ± 4.54 | −1.1 ± 1.81 | 14.8 ± 4.67 | 15.3 ± 4.25 | 0.4 ± 1.94 | 0.017 |
| questions | | | | | | | |

Quantitative results of C-peptide treatment: Table E17 also shows the average score points for each of the ten questions and the changes that had developed at the end of the study. Considering the results for all questions, C-peptide treated subjects were found to have improved by 0.7±3.2 points on average as compared to the placebo-treated patients, who had deteriorated by 1.5±3.8 points (P<0.066). For individual questions, the improvement was significantly different between C-peptide and placebo treated patients in the direction of a positive treatment effect as regards ability to penetrate (P<0.04) and maintenance of erection (P<0.005). For nine of the ten questions, the difference was towards improvement but it did not attain statistical significance.

Dichotomized results: The relative number of patients in the C-peptide and placebo treated groups, respectively, that reported improvement in response to the different questions is presented in Table E18. The percentage of patients with improvements was greater in the C-peptide treated group (P<0.008) when all questions were considered together.

TABLE E18
Percentage of patients reporting improvement in
response to the different questions in the C-peptide and
placebo treated groups

| Question | | Placebo | C-peptide |
|---|---|---|---|
| Q1 | Erection confidence | 18.2 | 35.9 |
| Q2 | Penetration | 0.0 | 17.9 |
| Q3 | Erection maintenance | 9.1 | 20.5 |
| Q4 | Erection duration | 0.0 | 17.9 |
| Q5 | Satisfaction | 27.3 | 35.9 |
| Q6 | Sexual desire | 9.1 | 30.8 |
| Q7 | Premature ejaculation | 9.1 | 15.4 |
| Q8 | Ejaculation failure | 25.0 | 16.7 |
| Q9 | Relation partner | 0.0 | 12.8 |
| Q10 | Partner's view | 0.0 | 12.8 |

C-peptide vs placebo P < 0.008

None of the individual questions yielded statistically significant positive results even though the responses to nine of the ten questions reported improvements for the patients on C-peptide. When for each subject the number of improvements in response to the ten questions were summed, it was found that on average the C-peptide treated patients reported improvement in response to 2.3±1.7 questions, whereas the corresponding number for placebo subjects was 0.9±1.0 (P<0.03).

Improvement in overall sexual function as reflected by the summed responses to all ten questions tended to be less marked in patients with retinopathy (P<0.06) and was found to be negatively related to altered heat perception on the tibia (P<0.04). Improvement in sexual function was not found to be related to study outcome for somatic nerve function as reflected by neurophysiological parameters, quantitative sensory testing or clinical scoring of peripheral neuropathy.

Erectile dysfunction: The first four questions were taken to reflect ED. While 44% of the patients were found to be afflicted with ED at the onset of the study (summed score of less than 16), the corresponding number at study end was 42%. Similarly, 51% of patients treated with C-peptide were affected by ED before the study as compared to 46% at end of the study. When the differences in response to treatment were summed for the four questions it was found that patients on C-peptide had improved by on average 0.4±1.9 score points in contrast to a deterioration of 1.1±1.8 points in the placebo treated patients (P<0.017) (Table E17). The relative numbers of patients reporting improvement or unchanged/deteriorated function in the two treatment groups are shown in Table E19. Thus, 46% of C-peptide treated patients experienced improvement of erectile function as compared to 9% of subjects receiving placebo (P<0.035).

TABLE E19
Improvement of erectile dysfunction upon C-peptide therapy

| Erectile function | | Placebo | C-peptide | Total |
|---|---|---|---|---|
| Improved | n | 1 | 18 | 19 |
| | | 9% | 46% | |
| Unchanged or | n | 10 | 21 | 31 |
| deteriorated | | 91% | 54% | |
| Total | n | 11 | 39 | 50 |

C-peptide vs placebo P < 0.035

There was no significant difference in ED improvement between patients on the low or the high dose of C-peptide. Improved ED status was found to be negatively correlated to abnormal heat perception on the foot and tibia (P<0.003-0.002), to abnormal vibration perception on the foot (P<0.008) and to clinical signs of abnormal clinical neurological findings on the lower leg (P<0.005).

C-Peptide Concentrations

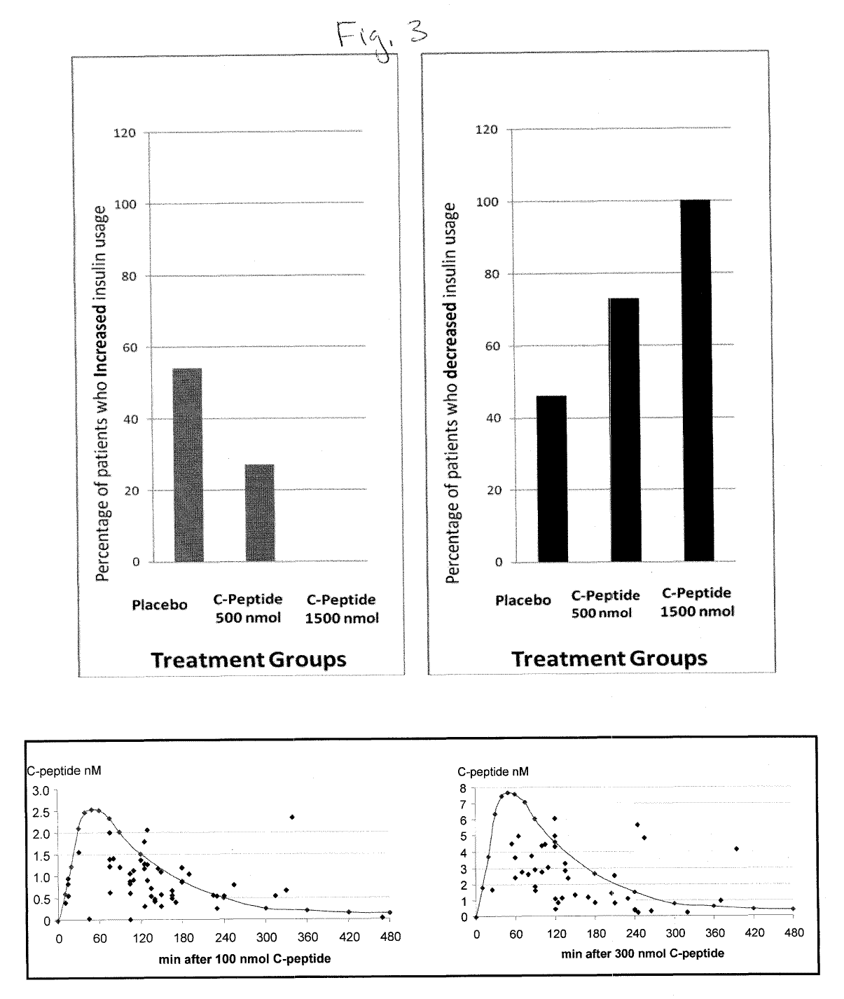

C-peptide plasma levels were measured at baseline, and after 3 and 6 months of treatment. Analysis was performed by the Department of Clinical Chemistry Karolinska University Hospital, Solna using a time-resolved fluoroimmunoassay (AutoDelfia, Wallac Oy, Turku, Finland). Compared to data generated in an earlier pharmacokinetic study, the concentrations obtained in the present study are slightly lower than expected (FIG. 3), but the results demonstrate similar variability as for, e.g., insulin when administered by the patients under everyday-like conditions.

C-peptide plasma levels were also measured pre-dosing in the morning in 22 randomly selected patients at the Karolinska Hospital after 4.5 months of study treatment (visit 3). As compared to the C-peptide concentration at baseline, the C-peptide plasma level at this visit was unchanged in the placebo-treated patients (0±0.008 nmoL/L; n=10), increased by 0.045±0.092 nmoL/L in the low-dose C-peptide group (n=8) and increased by 0.13±0.083 nmoL/L in the high-dose C-peptide group (n=4). Steady state C-peptide plasma levels ranged from about 0.2 nM to about 2.5 nM in the low dose C-peptide group and ranged from about 0.2 to about 6 nM in the high dose C-peptide group (FIG. 3).

Although the patient number is very small, it must be concluded that there is likely no significant accumulation occurring following repeated C-peptide dosing in the dose ranges employed in the present study.

Change from Baseline (BL) to 6 Months Treatment (Visit 4) in Neurophysiological Assessments

Following C-peptide administration for 6 months there was significant improvement of SCV in the active groups (low- and high-dose C-peptide groups combined), but this change was not significantly different from that of the placebo group (Table E21). Analyzing the numbers of responders, defined as a patient with an improvement in SCVp of >1 m/s, in each treatment group, there were 19% in the placebo group vs. 37% in the active group (p<0.0317; Table E20). There was no statistically significant difference between the responses in the low- and high-dose C-peptide groups.

Change from Baseline (BL) to 6 Months Treatment (Visit 4) in Neurophysiological Assessments

TABLE E20
Responders per treatment group. A responder is defined
as patients with improvement in SCVp of >1 m/s from
baseline, an improvement suggested to be of clinical
significance, taking study duration into consideration
(DCCT Group 1995a).

| | C-peptide groups | Placebo group |
|---|---|---|
| | n = 92 | n = 47 |
| Numbers of responders | 34 | 9 |
| Responders per treatment group | 37.0%* | 19.1% |

*p < 0.0317, C-peptide groups vs. placebo group with Pearson Chi-square test.

TABLE E21
Change from baseline to 6 months treatment (mean and median differences) in
neurophysiological function (PP data set). Improvements in MCV, SCV and
CMAP, SNAP are indicated by positive differences.

| | Low-dose | | High-dose | | | | | |
|---|---|---|---|---|---|---|---|---|
| | C-peptide | | C-peptide | | Placebo | | | |
| | n = 53 | | n = 39 | | n = 47 | | p < | |
| | Mean ± | | Mean ± | | Mean ± | | C- | p < |
| | SD | | SD | | SD | | peptide | Low- vs. |
| | [p < | | [p < | | [p < | | groups | high- |
| | within | Median | within | Median | within | Median | vs. | dose |
| | group] | (min; max) | group] | (min; max) | group] | (min; max) | placebo | groups |
| SCVp (m/s) | 0.38 ± 1.95 | 0.48 | 0.63 ± 1.65 | 0.30 | 0.24 ± 1.87 | 0.33 | ns | ns |
| | [ns] | (−6.12; 4.0) | [0.028] | (−2.33; 5.80) | [ns] | (−3.57; 8.18) | | |
| SCVi (m/s) | 0.77 ± 3.13 | 0.52 | 1.14 ± 2.23 | 1.03 | 0.75 ± 2.62 | 1.25 | ns | ns |
| | [0.052] | (−9.85; 8.22) | [0.004] | (−3.45; 6.45) | [ns] | (−4.15; 9.65) | | |
| SNAP (μV) | −0.06 ± 1.21 | −0.13 | 0.09 ± 1.30 | 0.13 | 0.24 ± 1.54 | 0.05 | ns | ns |
| | | (−2.80; 3.93) | | (−2.95; 4.70) | | (−3.03; 7.03) | | |
| MCV (m/s) | −0.25 ± 1.39 | −0.18 | −0.57 ± 1.53 | −0.70 | −0.53 ± 1.48 | −0.25 | ns | ns |
| | [ns] | (−4.35; 2.15) | [0.026] | (−3.70; 2.98) | [0.034] | (−5.0; 2.13) | | |
| CMAP (mV) | −0.15 ± 0.49 | −0.13 | −0.17 ± 0.72 | −0.07 | −0.15 ± 0.53 | −0.08 | ns | ns |
| | | (−1.33; 0.88) | | (−2.23; 1.15) | | (−2.35; 0.75) | | |
| MDL (ms) | 0.05 ± 0.44 | 0.03 | −0.0 ± 0.22 | 0.0 | 0.01 ± 0.46 | 0.05 | ns | ns |
| | | (−1.20; 1.10) | | (−0.45; 0.65) | | (−0.85; 1.55) | | |

ns = not significant

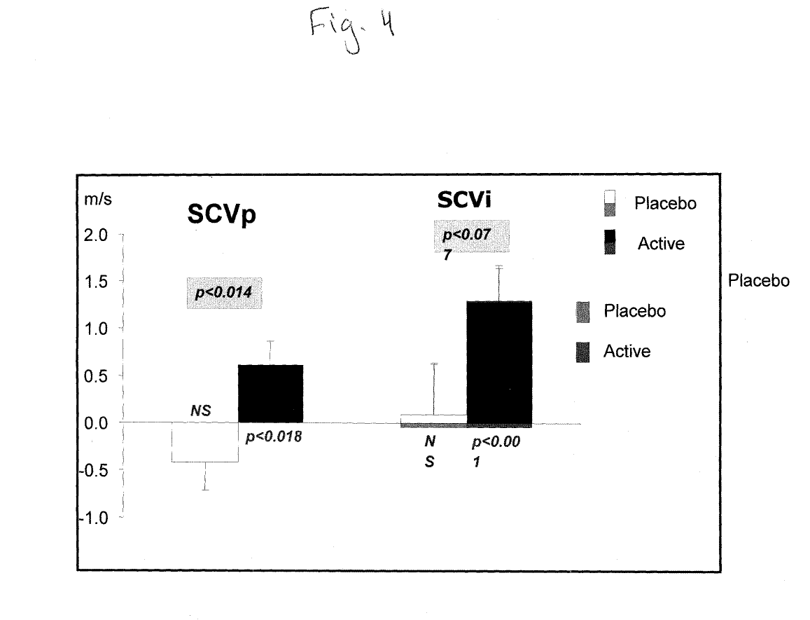

With a study duration as short as 6 months it is conceivable that patients who were less affected with neuropathy at baseline have a greater potential for an improvement during this relatively short treatment period. Thus, a subgroup analysis was performed in the subset of patients with less affected SCVp at baseline (SCVp>−2.5 SD, n=70). In this group, C-peptide induced an improvement of SCVp amounting to 1.03 m/s greater than that of the placebo group (C-peptide: 0.61 m/s; placebo: −0.42 m/s; p<0.015), FIG. 4; Table E22. In this group of patients there were no statistically significant differences in response to C-peptide in low and high doses.

TABLE E22
Change from baseline to 6 months treatment (mean and median differences) in
neurophysiological assessments in the subgroup of patients with SCVp > −2.5 SD
at baseline, treated with low-dose C-peptide, high-dose C-peptide, and placebo.

| | Low-dose | | High-dose | | | | | |
|---|---|---|---|---|---|---|---|---|
| | C-peptide | | C-peptide | | Placebo | | p < | p < |
| | n = 31 | | n = 18 | | n = 21 | | C- | Low- |
| | Mean ± | | Mean ± | | Mean ± | | peptide | vs. |
| | SD | | SD | | SD | | groups | high- |
| | [p < within | Median | [p < within | Median | [p < within | Median | vs. | dose |
| | group] | (min-max) | group] | (min-max) | group] | (min-max) | placebo | groups |
| SCVp | 0.46 ± 1.94 | 0.68 | 0.86 ± 1.33 | 0.63 | −0.42 ± 1.32 | −0.05 | 0.014 | ns |
| (m/s) | [ns] | (−3.23; 4.0) | [0.004] | (−2.27; 3.27) | [ns] | (−3.57; 1.45) | | |
| SCVi (m/s) | 1.13 ± 2.76 | 0.88 | 1.53 ± 2.19 | 2.16 | 0.07 ± 2.51 | 0.60 | 0.077 | ns |
| | [0.032] | (−5.25; 6.30) | [0.010] | (−3.45; 4.50) | [ns] | (−3.50; 3.63) | | |
| SNAP (μV) | 0.08 ± 1.43 | −0.13 | 0.47 ± 1.69 | 0.40 | 0.90 ± 1.74 | 0.47 | ns | ns |
| | [ns] | (−2.80; 3.93) | [ns] | (−2.95; 4.70) | [ns] | (−1.95; 7.03) | | |
| MCV (m/s) | −0.34 ± 1.43 | −0.35 | −0.65 ± 1.19 | −0.77 | −0.55 ± 1.56 | −0.45 | ns | ns |
| | [ns] | (−4.35; 2.15) | [0.049] | (−3.10; 1.28) | [ns] | (−5.0; 1.88) | | |
| CMAP | −0.13 ± 0.54 | −0.10 | −0.26 ± 0.78 | −0.29 | −0.11 ± 0.55 | −0.07 | ns | ns |
| (mV) | [ns] | (−1.33; 0.88) | [ns] | (−2.23; 1.15) | [ns] | (−1.68; 0.75) | | |
| MDL (ms) | 0.05 ± 0.44 | 0.02 | −0.05 ± 0.27 | −0.10 | 0.05 ± 0.38 | 0.15 | ns | ns |
| | [ns] | (−0.80; 1.10) | [ns] | (−0.45; 0.65) | [ns] | (−0.60; 0.72) | | |

ns = not significant

Change from Baseline (BL) to 6 Months Treatment (Visit 4) in Quantitative Sensory Function Assessments

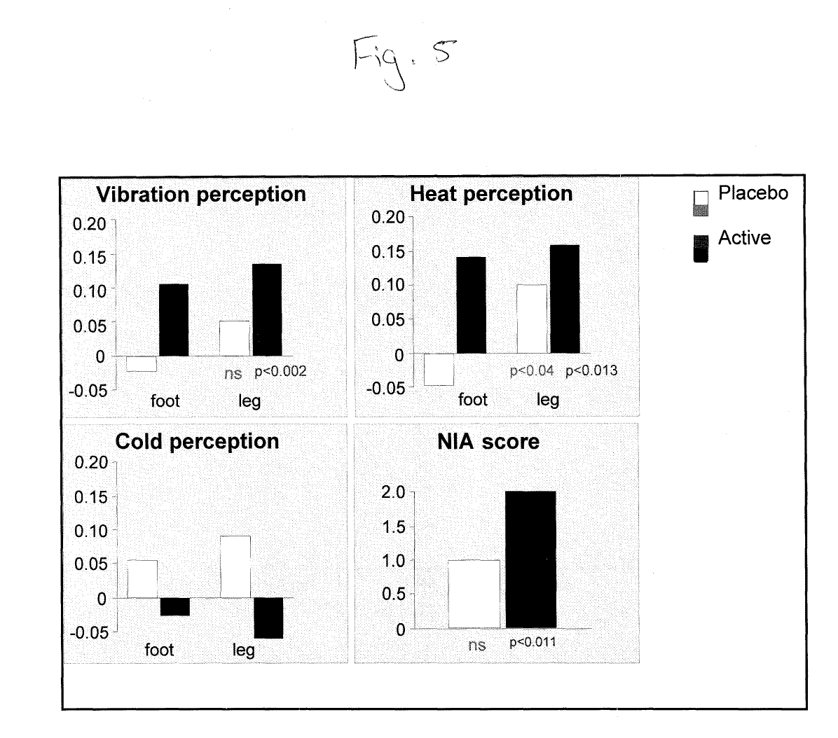

Following C-peptide administration, there was a tendency towards an improvement in the C-peptide-treated groups for vibration and heat perception as compared to placebo, but it did not reach statistical significance (Table E23; FIG. 5). C-peptide did not have an effect on cold perception.

TABLE E23
Change from baseline to 6 months treatment (mean and median differences) in
quantitative sensory function (PP data set). Improvements in QST are indicated by negative differences.

| | Low-dose | | High-dose | | | | | |
|---|---|---|---|---|---|---|---|---|
| | C-peptide | | C-peptide | | Placebo | | | |
| | n = 53 | | n = 39 | | n = 47 | | p < | p < |
| | Mean ± | | Mean ± | | Mean ± | | C- | Low- |
| | SD | | SD | | SD | | peptide | vs. |
| | [p < | | [p < | | [p < | | groups | high- |
| | within | Median | within | Median | within | Median | vs. | dose |
| | group] | (min; max) | group] | (min; max) | group] | (min; max) | placebo | groups |
| Cold threshold | 0.14 ± 1.33 | −0.02 | −0.18 ± 1.27 | 0.04 | −0.10 ± 0.86 | −0.06 | ns | ns |
| foot (SDS) | [ns] | (−3.04; 4.73) | [ns] | (−3.34; 2.10)[ns] | | (−1.71; 1.61) | | |
| Cold threshold | 0.10 ± 1.13 | 0.19 | −0.12 ± 1.06 | 0.01 | −0.07 ± 0.94 | −0.09 | ns | ns |
| lower leg (SDS) | [ns] | (−2.67; 3.40) | [ns] | (−2.18; 2.47) | [ns] | (−1.93; 2.0) | | |
| Heat threshold | −0.08 ± 0.59 | −0.22 | −0.10 ± 0.81 | −0.01 | −0.02 ± 0.61 | 0.05 | ns | ns |
| foot (SDS) | [ns] | (−1.42; 1.15) | [ns] | (−2.44; 1.60) | [ns] | (−1.30; 1.34) | | |
| Heat threshold | −0.30 ± 0.74 | −0.26 | −0.07 ± 0.82 | −0.12 | −0.21 ± 0.63 | −0.10 | ns | ns |
| lower leg (SDS) | [0.006] | (−1.79; 1.31) | [ns] | (−2.09; 2.24) | [0.041] | (−1.66; 1.30) | | |
| Vibration | −0.07 ± 0.68 | −0.11 | −0.12 ± 0.69 | −0.10 | −0.03 ± 0.86 | 0.02 | ns | ns |
| threshold foot | [ns] | (−1.42; 2.18) | [ns] | (−1.93; 0.92) | [ns] | (−2.75; 2.16) | | |
| (SDS) | | | | | | | | |
| Vibration | −0.18 ± 0.46 | −0.09 | −0.22 ± 0.79 | −0.24 | −0.09 ± 0.57 | −0.05 | ns | ns |
| threshold lower | [0.013] | (−1.61; 0.68) | [0.057] | (−2.64; 2.18) | [ns] | (−1.97; 1.17) | | |
| leg (SDS) | | | | | | | | |

ns = not significant

Change from Baseline (BL) to 6 Months Treatment (Visit 4) in Neurological Impairment Assessments and Symptoms

Following C-peptide administration for 6 months there was a significant improvement in total NIA score. The score was decreased by −2.16 points (both C-peptide groups), but this change was not statistically significantly different from that in the placebo group (−0.94 point), FIG. 5. No significant changes in symptoms were observed during the study. See Table E24.

TABLE E24
Change from baseline to 6 months treatment (mean and median differences) in
neurological assessments and symptoms (PP data set). Improvements in
NIA and symptom assessment (SA) are indicated by negative differences.

| Low-dose | | High-dose | | | | | |
|---|---|---|---|---|---|---|---|
| C-peptide | | C-peptide | | Placebo | | | |
| n = 53 | | n = 39 | | n = 47 | | p < | p < |
| Mean ± | | Mean ± | | Mean ± | | C- | Low- |
| SD | | SD | | SD | | peptide | vs. |
| [p < | | [p < | | [p < | | groups | high- |
| within | Median | within | Median | within | Median | vs. | dose |
| group] | (min; max) | group] | (min; max) | group] | (min; max) | placebo | groups |

Neurological assessments

| | | | | | | | | |
|---|---|---|---|---|---|---|---|---|
| Total NIA score | −1.74 ± 7.06 | −2.0 | −2.75 ± 8.18 | −2.0 | −0.94 ± 6.45 | −1.0 | ns | ns |
| | [ns] | (−18.0; 16.0) | [0.043] | (−38.0; 10.0) | [ns] | (−15.0; 17.0) | | |
| Big toe score | −0.38 ± 3.56 | 0 | −1.32 ± 3.80 | −1.0 | −0.37 ± 3.25 | 0 | ns | ns |
| | | (−9.0; 8.0) | | (−14.00; 6.0) | | (−8.0; 6.0) | | |
| Foot score | −0.88 ± 2.88 | 0 | −1.24 ± 3.65 | 0 | −0.16 ± 2.75 | 0 | ns | ns |
| | | (−8.0; 7.0) | | (−14.0; 7.0) | | (−7.0; 6.0) | | |
| Lower leg | −0.55 ± 1.91 | 0 | −0.55 ± 2.65 | 0 | −0.96 ± 2.15 | 0 | ns | ns |
| | | (−5.0; 4.0) | | (−12.0; 4.0) | | (−5.0; 6.0) | | |

Symptom assessment

| | | | | | | | | |
|---|---|---|---|---|---|---|---|---|
| Sum Symptom | −0.02 ± 1.03 | 0.0 | −0.13 ± 1.22 | 0.0 | −0.07 ± 0.68 | 0.0 | ns | ns |
| Score | [ns] | (−3.0; 3.0) | [ns] | (−5.0; 3.0) | [ns] | (−2.0; 2.0) | | |

ns = not significant

Change from Baseline (BL) to 6 Months Treatment (Visit 4) in Autonomic Nerve Function

The change in E/I ratio is presented in Table E25. There was no significant effect on the E/I ratio in any of the three treatment groups. In the subgroup of patients with pathological E/I ratio at baseline, the change in E/I ratio was on average 1% (not significant).

TABLE E25
Change from baseline to 6 months treatment (mean and median differences) in
autonomic nerve function (ITT data set), excluding patients on concomitant beta-blockers.
Improvements in E/I ratio are indicated by positive differences.

| | Low-dose C- | | High-dose | | | | | |
|---|---|---|---|---|---|---|---|---|
| | peptide | | C-peptide | | Placebo | | | |
| | n = 50 | | n = 48 | | n = 48 | | p < | p < |
| | Mean ± | | Mean ± | | Mean ± | | C- | Low- |
| | SD | | SD | | SD | | peptide | vs. |
| | [p < | | [p < | | [p < | | groups | high- |
| | within | Median | within | Median | within | Median | vs. | dose |
| | group] | (min; max) | group] | (min; max) | group] | (min; max) | placebo | groups |
| E/I ratio | −0.01 ± 0.06 | 0.00 | 0.01 ± 0.07 | 0.00 | 0.02 ± 0.13 | 0.00 | ns | ns |
| | [ns] | (−0.22; 0.11) | [ns] | (−0.24; 0.21) | [ns] | (−0.09; 0.84) | | |

ns = not significant

ECG and Vital Signs

There were no clinically significant abnormalities in ECG reported during the study medication treatment, nor were there any significant changes in heart rate, blood pressure, and QTc in any of the three treatment groups (Table E26). However, there was a significantly greater decrease in systolic blood pressure in the high-dose C-peptide group compared to the low-dose C-peptide group (p<0.004), but this decrease was not statistically different from that in the placebo group.

TABLE E26
Change from baseline to 6 months treatment (mean and median differences) in ECG and
vital signs recording (visit 4) (PP data set). A reduction in QTc is indicated by a negative difference.

| | Low-dose C- | | High-dose C- | | | | |
|---|---|---|---|---|---|---|---|
| | peptide | | peptide | | Placebo | | |
| | n = 53 | | n = 39 | | n = 47 | | |
| | | Median | | Median | | Median | |
| | Mean ± | (min- | Mean ± | (min- | Mean ± | (min- | |
| | SD | max) | SD | max) | SD | max) | p < |
| Heart rate (bpm) | −0.9 ± 8.2 | −2.0 | −3.2 ± 10.0 | −4.0 | −0.8 ± 9.4 | −2.0 | ns |
| | | (−20; 22) | | (−22; 19) | | (−20; 28) | |
| QTc-interval (ms) | −0.1 ± 17.5 | 0.0 | −4.5 ± 20.3 | 0.0 | −2.0 ± 23.5 | 0.0 | ns |
| Bazett | | (−48; 47.2) | | (−58; 36.6) | | (−44; 100) | |
| QTc-interval (ms) | 0.9 ± 14.1 | 1.3 | −0.5 ± 16.2 | 0.0 | −1.1 ± 18.6 | −3.3 | ns |
| Fredericia | | (−43; 41.1 | | (−44; 38.2) | | (−31; 95.9) | |
| BPs (mmHg) | −2.1 ± 12.8 | −1.0 | −8.8 ± 10.0 | −8.0 | −4.8 ± 12.2 | −4.0 | ns |
| | | (−40; 22) | | (−34; 15) | | (−41; 27) | |
| BPd (mmHg) | −2.7 ± 8.2 | −1.0 | −2.6 ± 8.0 | −1.0 | −3.1 ± 8.0 | −5.0 | ns |
| | | (−28; 20) | | (−22; 11) | | (−20; 15) | |

ns = not significant;
BPs = systolic blood pressure;
BPd = diastolic blood pressure

### Assessment of Antibody Formation Against C-Peptide

Samples for the assessment of antibody formation were collected at baseline and at visits 2 and 4. An appropriate enzyme-linked immunoabsorbant assay (ELISA) method was developed and assessments carried out. The results indicate no or very weak reactivity to C-peptide both at baseline and after 3 and 6 months of C-peptide treatment.

Compliance

The assessment of compliance was based on the integrated information obtained from the patient's diary with recordings of drug administration, by the visual control of returned unused and used vials and by questioning about any handling problems according to predetermined questions at clinical visits 1, 2, 3, and 4. Compliance was also checked after closure of the database by qualitative analysis of C-peptide in blood samples taken at visits 2 and 4. The final assessment as to the individual patient's compliance was made at the clean file meeting. Compliance was considered sufficient, i.e., >80% of the intended total study dose had been administered, in all patients but one in the high-dose C-peptide group and one in the low-dose C-peptide group. They were considered major violators and their data were excluded from the PP analysis of efficacy.

Statistical/Analytical Issues

Adjustments for Covariates

An improvement of the subject's glycemic control (HbA1c) was expected in all treatment groups and this change was evaluated. Exploratory multivariate analyses including possible predictors of the dependent variables were performed on the primary efficacy variable (change baseline to 6 months) using HbA1c as one of the predictors. There were no correlations found between change in glycemic control and change in SCV.

Handling of Dropouts or Missing Data

In the statistical calculations in the ITT population, last-value-carried-forward (LVCF) was used for missing data.

Multicenter Studies

Patients were recruited from 5 sites in Sweden: site 1. Karolinska Hospital, Stockholm; site 2. Lundby Hospital/Sahlgrenska University Hospital, Gothenburg; site 4. Linköpings University Hospital; site 5. Uppsala University Hospital; and site 6. Huddinge University Hospital (site 3 withdrew their participation already before the study start). Potential “site-differences” were evaluated using multivariate analyses. There were no differences in study conduct, patient characteristics, or clinical settings with respect to response between the sites.

Use of an “Efficacy Subset” of Patients

The analyses of efficacy (primary and secondary variables) were performed in the PP data set and in those of the patients in the PP data set who had SCVp>−2.5 SD at baseline. The basis for the selection of this latter group rests with the possibility to influence severely affected nerves during a relatively short treatment period—with study duration as short as 6 months it is conceivable that patients who were less affected with neuropathy at baseline have a greater potential for an improvement during a relatively short treatment period. Thus, a subgroup analysis was performed in the subset of the patients, with less affected SCVp at baseline.

Drug-Drug and Drug-Disease Interactions

The exclusion criteria specifically excluded concurrent therapies with drugs, which potentially might interact with the effect of the investigational drug, i.e., Ca²⁺-channel blockers. The study design and total number of patients in the study do not allow any conclusions as to a relationship between response and past and/or current illness or ongoing concomitant medication.

Efficacy Conclusions

Diabetic Neuropathy

It is concluded that C-peptide given in therapeutic doses (500-1500 nmoL/day) for 6 months exerts a beneficial effect on nerve function in patients with early stage neuropathy. The higher dose level (3 times the lower dose) did not result in a statistically significantly greater effect. The present study confirms and extends previous findings to a group of patients with manifest diabetic neuropathy.

Erectile Dysfunction

It is concluded that C-peptide given in therapeutic doses (500-1500 nmoL/day) for 6 months exerts a beneficial effect on sexual function in male patients with type 1 diabetes and peripheral neuropathy. Significantly this patient group often fails to respond to phosphodiesterase (PDE-5) inhibitors such as Viagra, suggesting that the current results, which demonstrate for the first time the beneficial effects of C-peptide therapy on sexual dysfunction represents a major contribution to addressing this unmet medical need.

Indeed it is possible that the unique mechanism of action of C-peptide may facilitate improved therapies for erectile dysfunction in combination with PDE-5 inhibitors such as Viagra. Specifically it is known based on in vitro data that C-peptide can under in vitro conditions stimulate NO production, and can result in the induction of endothelial nitric oxide synthase in tissue culture cells. Moreover, C-peptide has been shown to increase parasympathetic nerve activity in rats and to enhance heart rate variability during breathing in type 1 diabetes patients. While the relative contributions of increased blood flow and augmented parasympathetic nerve activity cannot be readily determined in present study, both changes would be expected to further augment the effect of a PDE-5 inhibitor because they act by different mechanisms, i.e. would be expected to increase NO production, and not just prevent its degradation by PDE-5 action.

IV. Safety Evaluation

Extent of Exposure

The low-dose regimen was expected to give a mean physiological plasma concentration of ˜1 nmoL/L during the 24 h period. The high dose regimen was expected to result in concentrations ˜3 times those of the low dose regimen. The actual exposure is described in section above on “C-peptide concentrations”. Blood samples for determination of C-peptide in plasma were drawn at the S/B visit, visit 2, and at end of study visit 4. The sampling of C-peptide in plasma at visits 2 and 4 coincided with the clinical visit and the time between the most recent administration of the trial drug and sampling varied between patients. Thereby, the results of C-peptide concentrations in plasma became less suitable for pharmacokinetic evaluations.

Adverse Events (AEs)

### Brief Summary of Adverse Events

Fifty-two (52) patients in the high-dose C-peptide group, 56 patients in the low-dose C-peptide group and 53 patients in the placebo group (ITT data set) were evaluated for safety.

No deaths were reported during the study. There were two SAEs reported during the study, and one patient withdrew from the study treatment due to an AE. In total, there were 173 AEs reported in 48 out of 56 patients (86%) in the low-dose C-peptide group, 154 AEs in 44 out of 52 patients (85%) in high-dose C-peptide group, and 166 AEs reported in 44 out of 53 patients (83%) in the placebo group. The most frequently reported AEs were headache and nasopharyngitis, both equally occurring between the three treatment groups (Table E27).

### Display of Adverse Events

TABLE E27
Treatment emergent AEs of any severity listed by SOC and
preferred term (PP data set). Numbers in parentheses denote
numbers of AEs, in judgment of the investigator,
probably/possibly was related to study drug.

| | | Treatment | | | | | |
|---|---|---|---|---|---|---|---|
| | | Low-dose | | High-dose | | | |
| SOC | Preferred Term | C-peptide | | C-peptide | | Placebo | |
| Blood and lymphatic | Anemia | 1 | | 1 | | | |
| system disorder | Hemorrhage | | | 1 | | | |
| Cardiac disorder | Cardiac arrhythmia (SVES) | 1 | | | | | |
| | ST elevation (ECG) | 1 | | | | | |
| | Tachycardia (stress related) | 1 | | | | | |
| Eye disorder | Dacryocanaliculitis | 1 | | | | | |
| | Macular edema | | | 1 | | | |
| | Retinopathy proliferative | | | | | 1 | |
| | Vitreous hemorrhage | | | 1 | | | |
| | Vision decreased | 1 | | | | | |
| Gastrointestinal | Abdominal pain | | | 3 | | 1 | |
| Disorders | Aptyalism | | | 1 | | | |
| | Celiac disease | 1 | | | | | |
| | Constipation | | | | | 1 | |
| | Diarrhea | 2 | | 1 | | 3 | |
| | Dysphagia | | | | | 1 | |
| | Flatulence | 1 | | | | | |
| | Gastritis | | | 1 | | 1 | |
| | Nausea | 1 | | 4 | | | |
| | Reflux gastritis | 2 | | | | | |
| | Tooth ache | 2 | | 3 | | 2 | |
| General disorders and | Pyrexia | 3 | | | | 3 | |
| administration site | Chest pain | | | 2 | | | |
| conditions | Fatigue | | | 2 | | | |
| | Feeling abnormal | | | 3 | | 4 | (1) |
| | Injection site burning | | | 1 | (1) | | |
| | Injection site hemorrhage | | | | | 2 | (1) |
| | Peripheral edema | | | | | 3 | |
| Immune system | Allergic urticaria | 1 | | | | | |
| disorders | Dermatitis allergic | 1 | | | | 1 | |
| | Rheumarthritis | 1 | | | | | |
| | Season allergy | 1 | | | | | |
| Infections and | Acute HIV infection | 1 | | | | | |
| infestations | Borrelia infection | 1 | | | | | |
| | Bronchitis | 1 | | | | | |
| | Candidiasis | 2 | | | | | |
| | Eye infection | | | | | 2 | |
| | Gastroenteritis | 2 | | 7 | | 2 | |
| | Herpes simplex | 1 | | | | | |
| | Herpeszoster | 1 | | | | | |
| | Influenza | 1 | | | | 4 | |
| | Nasopharyngitis | 31 | | 23 | | 26 | |
| | Otitis | 1 | | 2 | | | |
| | Pharyngitis | 1 | | 2 | | 3 | |
| | Perianal abscess | | | | | 1 | |
| | Rhinitis | | | | | 1 | |
| | Sinusitis | 4 | | 2 | | 2 | |
| | Sepsis | | | 1 | | | |
| | Skin infection | 5 | | 1 | | | |
| | Tonsilitis | 1 | | 2 | | 1 | |
| | Tooth infection | | | 2 | | 1 | |
| | Upper respiratory tract | 3 | | 1 | | 3 | |
| | infection | | | | | | |
| | Urinary tract infection | 3 | | 1 | | 6 | |
| Injury poisoning, | Alcohol poisoning | | | | | 1 | |
| procedural complications | Blister | | | 2 | | | |
| | Dizziness | | | 1 | | | |
| | Eye injury | | | 1 | | | |
| | Food poisoning | | | 1 | | | |
| | Joint sprain ankle | 1 | | 1 | | 1 | |
| | Knee injury | | | 1 | | | |
| | Pain in extremity | | | | | 1 | |
| | Rib fracture/injury | 1 | | 2 | | | |
| | Scar | | | 1 | | | |
| | Skin injury | 2 | | | | | |
| | Tooth injury | | | 1 | | | |
| Investigations | Cardiac murmur | | | 1 | | | |
| | Elevated serum creatinine | 1 | | | | | |
| | Hematuria | | | 1 | | | |
| | Hepatic enzyme increase | 2 | (1) | 2 | | 1 | |
| | Hypercalcemia | | | 1 | | | |
| | Hyperkalaemia | 1 | | | | 1 | |
| | Laparoscopy | | | | | 1 | |
| | Leucopenia | | | 1 | | | |
| | Lymph node enlargement | | | 1 | | | |
| | Weight decreased | | | | | 1 | |
| | Weight increased | 1 | | | | | |
| Metabolism and | Blood glucose fluctuation | | | | | 1 | |
| nutritional disorders | Hyperglycemia | 1 | (1) | 1 | | 1 | |
| | Hypoglycemia | 2 | (1) | 5 | (1) | 5 | |
| | Hypoglycemic coma | 2 | | 2 | | | |
| | Hyperlipidemia | 1 | | | | | |
| | Hypothyroidism | | | | | 1 | |
| | Increased appetite | | | | | 1 | |
| | Insulin sensitivity increased | 1 | (1) | | | 1 | (1) |
| | Vitamin B12 deficiency | | | 1 | | | |
| Musculoskeletal and | Arthralgia | 3 | | 3 | | 6 | (1) |
| connective tissue | Arthritis | 1 | | 1 | | | |
| disorder | Arthropathy | 2 | | 1 | | 1 | |
| | Back pain | | | 2 | | 4 | |
| | Epicondylitis | 1 | | 1 | | | |
| | Exostosis | 2 | | | | | |
| | Muscle spasm | | | 1 | | | |
| | Muscle cramp | 1 | | | | 1 | |
| | Muscular atrophy | | | 1 | | | |
| | Musculoskeletal chest pain | 1 | | | | | |
| | Myalgia | 3 | | 1 | | | |
| | Pain in extremity | 3 | (1) | 4 | | 8 | (1) |
| | Pain in jaw | | | | | 1 | |
| | Pain in neck | 1 | | | | 1 | |
| | Pain in shoulder | | | 1 | | | |
| | Periarthritis | | | | | 1 | |
| | Synovial disorder | 1 | | | | | |
| | Tendinitis | | | 1 | | | |
| | Tendinous contracture | 1 | | | | | |
| | Tendosynovitis stenosans | | | 1 | | | |
| | Torticollis | | | 1 | | | |
| Nervous system | Burning sensation | | | 1 | | | |
| disorders | Carpal tunnel syndrome | 1 | | | | 1 | |
| | Dizziness | 2 | | | | 1 | |
| | Headache | 35 | | 17 | | 31 | (3) |
| | Hyperaesthesia | | | 1 | (1) | | |
| | Hyperthermeasthesia | 2 | (2) | | | | |
| | Hypoaesthesia | 2 | | | | | |
| | Insomnia | | | | | 1 | (1) |
| | Migraine | | | 1 | | | |
| | Multiple sclerosis | 1 | | | | | |
| | Pain in extremity | | | | | 1 | (1) |
| | Paraesthesia | 2 | (1) | 2 | (2) | 2 | (2) |
| | Restless legs syndrome | | | 1 | | | |
| | Sciatica | | | 1 | | | |
| | Sensory loss | | | 1 | | 2 | |
| | Vertigo | | | 1 | | | |
| Pregnancy, puerperium | Pregnancy | | | 2 | | | |
| Psychiatric disorders | Depression | 1 | | | | 1 | |
| | Panic disorder | 1 | | | | | |
| | Stress disorder | 1 | | | | | |
| Renal and urinary | Nephritis | | | | | 1 | |
| Reproductive system | Dysmenorrhea | | | | | 1 | |
| and breast disorders | Menopausal disorder | | | 1 | | 1 | |
| | Menstration irregular | | | 1 | | 1 | |
| Respiratory, thoracic, | Asthma | 1 | | | | | |
| | Cough | | | 1 | | 1 | |
| | Epistaxis | | | 1 | | 3 | |
| Skin and subcutaneous | Dry skin | 1 | | | | | |
| tissue disorders | Fibroma | 1 | | | | | |
| | Skin induration | 2 | | 1 | | | |
| | Skin infection | | | 2 | | | |
| | Sweating increased | | | 1 | | | |
| | Tinea | | | | | 1 | |
| Surgical and Medical | Eye operation | 1 | | | | | |
| | Limb operation | 1 | | 1 | | | |
| | Tooth extraction | | | 1 | | 1 | |
| Vascular disorders | Arterial disorder | 1 | | 1 | | | |
| | Hypertension | 1 | | 1 | | 1 | |
| | Hypotension | | | 1 | | | |

| | | | | | | |
|---|---|---|---|---|---|---|
| Total | 173 | (8) | 156 | (5) | 166 | (12) |

### Analysis of Adverse Events

AEs were reported in all but 8 patients in the low-dose C-peptide group, 6 patients in high-dose C-peptide group, and 9 patients in placebo group. A total number of 495 AEs were reported during the study. The numbers of AEs was similar in all dose groups within the different SOCs. The most frequently reported AE was headache, followed by nasopharyngitis, both equally occurring in all dose groups. One-hundred-fifty-five (155) AEs were reported as having moderate intensity (51 in low-dose C-peptide group, 43 in high-dose C-peptide group, and 61 in the placebo group) and 12 AEs were reported with severe intensity (1 in low-dose C-peptide group, 9 in high-dose C-peptide group, and 2 in the placebo group). A listing of these patients is shown in Table E28.

Twenty-five (25) AEs (8 in low-dose C-peptide group, 5 in high-dose C-peptide group and 12 in the placebo group) spread on different AE diagnosis, were in the opinion of the investigators reported as possible/probable related to the study drug. Hypoglycemic events were noted by the patient and were not reported as AEs, unless the severity of the event required assistance by another person or else in the opinion of investigator it should be reported as an AE.

TABLE E28
Treatment emergent AEs of moderate and severe intensity (ITT data set). S
(Severe), M (Moderate); followed by number of AE incidences in parentheses.

| | | Treatment | | |
|---|---|---|---|---|
| | | Low-dose | High-dose | |
| SOC | Preferred Term | C-peptide | C-peptide | Placebo |
| Cardiac disorder | ST elevation (ECG) | M (1) | | |
| Eye disorder | Dacryocanaliculitis | M (1) | | |
| | Retinopathy | | | M (1) |
| | proliferative | | | |
| | Vitreous hemorrhage | | M (1) | |
| | Vision decreased | M (1) | | |
| Gastrointestinal | Abdomnial pain | | M (1) | |
| Disorders | Celiac disease | M (1) | | |
| | Constipation | | | M (1) |
| | Diarrhea | | S (1) | M (1) |
| | Dysphagia | | | M (1) |
| | Gastritis | | M (1) | |
| | Nausea | M (1) | M (1) | |
| | Toothache | M (1) | M (2) | M (2) |
| General disorders | Pyrexia | M (1) | | M (2) |
| and | Fatigue | | M (1) | |
| administration | Feeling abnormal | | M (3) | M (1) |
| site conditions | Edema peripheral | | | M (1) |
| Infections and | Acute HIV infection | M (1) | | |
| infestations | Borrelia infection | M (1) | | |
| | Bronchitis | M (1) | | |
| | Gastroenteritis | | M (3) | M (1) |
| | Herpes zoster | M (1) | | |
| | Nasopharyngitis | M (6) | M (8) | M (7) |
| | Otitis | M (1) | | |
| | Pharyngitis | S (1) | M (1) | M (3) |
| | Perianal abscess | | | M (1) |
| | Sinusitis | M (4) | M (2) | M (1) |
| | Sepsis | | S (1) | |
| | Tonsillitis | M (1) | | M (1) |
| | Tooth infection | | | M (1) |
| | Urinary tract infection | | | M (2) |
| Injury poisoning, | Alcohol poisoning | | | M (1) |
| procedural | Dizziness | | M (1) | |
| complications | Knee injury | | M (1) | |
| | Pain in extremity | | | M (1) |
| | Rib fracture/injury | | M (1); S (1) | |
| Investigations | Hepatic enzyme | | S (1) | |
| | increase | | | |
| Metabolism and | Blood glucose | | | M (1) |
| nutritional | fluctuation | | | |
| disorders | Hyperglycemia | | S (1) | M (1) |
| | Hypoglycemia | M (2) | M (1); S (1) | M (1); S (2) |
| | Hypoglycemic coma | M (1) | S (2) | |
| | Hyperlipidemia | M (1) | | |
| | Increased appetite | | | M (1) |
| Musculoskeletal | Arthralgia | M (1) | M (1) | M (2) |
| and connective | Arthritis | M (1) | M (1) | |
| tissue dissorder | Arthropathy | M (1) | | |
| | Back pain | | M (1) | M (1) |
| | Epicondylitis | | M (1) | |
| | Muscle cramp | M (1) | | M (1) |
| | Myalgia | M (2) | | |
| | Pain in extremity | | M (2) | M (4) |
| | Pain in neck | | | M (1) |
| | Tendinous contracture | M (1) | | |
| Nervous system | Carpal tunnel | M (1) | | |
| disorders | syndrome | | | |
| | Dizziness | M (1) | | |
| | Headache | M (11) | M (3) | M (15) |
| | Migraine | | M (1) | |
| | Paraesthesia | | M (1) | |
| | Restless legs | | M (1) | |
| | syndrome | | | |
| | Sciatica | | M (1) | |
| Pregnancy, | Pregnancy | | S (1) | |
| puerperium and | | | | |
| perinatal | | | | |
| conditions | | | | |
| Psychiatric | Depression | M (1) | | M (1) |
| disorders | Stress disorder | M (1) | | |
| Renal and urinary | Nephritis | | | M (1) |
| disorder | | | | |
| Respiratory, | Epistaxis | | | M (1) |
| thoracic and | | | | |
| mediastinal | | | | |
| disorders | | | | |
| Skin and | Skin infection | M (1) | | |
| subcutaneous | | | | |
| tissue disorders | | | | |
| Surgical and | Limb operation | M (1) | M (1) | |
| Medical | Tooth extraction | | | M (1) |
| Vascular | Hypotension | | M (1) | |
| disorders | | | | |

| | | | |
|---|---|---|---|
| Total Adverse Events | Moderate = 51 | Moderate = 43 | Moderate = 61 |
| | Severe = 1 | Severe = 9 | Severe = 2 |

There were 14 treatment emergent symptomatic hypoglycemic episodes and two hypoglycemic coma episodes requiring external assistance.

Deaths, Other Serious Adverse Events, and Other Significant Adverse Events

There were no deaths in this trial. Two SAEs occurred during the study.

Analysis and Discussion of Deaths, Other Serious Adverse Events, and Other Significant Adverse Events

Two SAEs occurred during the study, both patients were treated with high-dose C-peptide (1,500 nmoL/24 h). The assessments of the events relationship to the trial medication were by the investigators at both sites “unlikely/not related”.

Clinical Laboratory Evaluation

There were generally very few deviations outside the normal range in most of the laboratory tests and when observed most of them were of minor clinical significance. Following the study treatment there were few shifts from normal to abnormal and there were no significant changes in any of the variables.

Vital Signs, Physical Findings, and Other Observations to Safety

There were no clinically significant abnormalities in ECG reported during the study medication treatment, nor were there any significant changes in heart rate, blood pressure, and QTc in any of the three treatment groups (Table E26).

Safety Conclusions

C-peptide was well tolerated in the doses of 500-1,500 nmoL/24 h and no adverse drug reactions or significant changes in safety variables (blood chemistries and vital signs) were observed during the study period. No local reactions were reported.

V. Discussion and Overall Conclusions

Overall Conclusions

Neuropathy

It is concluded that C-peptide given in a therapeutic dose for 6 months was well tolerated and had a beneficial effect on nerve function in patients with early stage neuropathy. Thus, C-peptide used as a complement to regular insulin therapy may provide an effective approach to the management of the long-term complications of type 1 diabetes such as neuropathy.

The primary variable in the present study, sensory nerve conduction velocity (SNCV), improved significantly within the group of patients receiving active treatment, but the difference in SNCV response after C-peptide therapy versus placebo treatment did not attain statistical significance. Consequently, the study did not meet its primary end point. However, further statistical analyses show that there were significantly more “responders” in the C-peptide-treated groups compared to the placebo group (p<0.03), responders being defined as patients with an improvement in SNCV greater than 1 m/s, an increase recognized by the DCCT study to be of clinical significance. In view of the exploratory nature of this trial, the result of the responder analysis can be viewed as a positive study outcome.

Subgroups

A subgroup of the patients was subjected to further statistical analysis. Thus, in the half of the patient group that showed the less severe nerve dysfunction (n=70), as evaluated from the degree of SNCV reduction at baseline, there was significant improvement in SNCV within the C-peptide treated group. In the corresponding placebo group there was minimal change in SNCV compared to baseline, and the improvement in the C-peptide groups was significantly greater than that in the placebo group. Moreover, analysis of the responders in this subgroup again demonstrated a significantly greater number of responders in the patients on active treatment compared to placebo. These findings confirm and extend the results from a previous 3 months phase II trial in which type 1 diabetes patients with early stage (sub-clinical) neuropathy were found to markedly improve their SNCV during C-peptide replacement therapy. The findings suggest that the beneficial effect of C-peptide—at least in studies with 3-6 months duration—is most pronounced in the less diseased patients, emphasizing the need for early intervention in this disorder.

Erectile Dysfunction

It is concluded that C-peptide given in therapeutic dose for 6 months is capable of exerting a beneficial effect on both erectile function and sexual satisfaction in men with type 1 diabetes. Significantly this patient group often fails to respond to existing treatments for erectile dysfunction suggesting that the beneficial effects of C-peptide therapy on sexual dysfunction demonstrated here represents a major contribution to addressing this unmet medical need. C-peptide's unique ability to modulate both parasympathetic autonomic neuronal function, as well as improved vasodilation suggests that C-peptide treatment may have utility for male erectile dysfunction, particularly in C-peptide deficient groups such as insulin-dependent patients. Importantly such treatments should enhance the effectiveness of the treatment of ED by PDE-5 inhibitors.

### REFERENCES FOR EXAMPLES SECTION

American Diabetes Association and American Academy of Neurology (1988). Report and recommendation of the San Antonio Conference on Diabetic Neuropathy (consensus statement). Diabetes Care 11: 592-597.
Dalla Vestra, M., A. Saller, E. Bortoloso, M. Mauer and P. Fioretto (2000). Structural involvement in type 1 and type 2 diabetic nephropathy. Diabetes Metab 26 (suppl 4): 8-14.
DCCT Group (1993). The effect of intensive treatment of diabetes on the development and progression of long-term complications in insulin-dependent diabetes mellitus. New Engl J. Med. 329: 977-983.
DCCT Group (1995a). Effect of intensive diabetes treatment on nerve conduction in the diabetes control and complications trial. Ann Neurol 38: 869-880.
DCCT Group (1995b). Effect of intensive therapy on the development and progression of diabetic nephropathy in the Diabetes Control and Complications Trial. The Diabetes Control and Complications (DCCT) Research Group. Kidney Int 47(6): 1703-1720.
Ekberg, K., T. Brismar, B.-L. Johansson, et al. (2003). Amelioration of sensory nerve dysfunction by C-peptide in patients with type 1 diabetes. Diabetes 52: 536-541.
Forst, T., T. Kunt, T. Pohlmann, et al. (1998). Biological activity of C-peptide on the skin microcirculation in patients with insulin dependent diabetes mellitus. J. Clin. Invest. 101: 2036-2041.
Hansen, A., B.-L. Johansson, J. Wahren and H. Bibra von (2002). C-peptide exerts beneficial effects on myocardial blood flow and function in patients with type 1 diabetes. Diabetes 51(10): 3077-3082.
Johansson, B.-L., K. Borg, E. Fernqvist-Forbes, et al. (2000). Beneficial effects of C-peptide on incipient nephropathy and neuropathy in patients with type I diabetes. Diabetic Medicine 17(3): 181-189.
Johansson, B.-L., K. Borg, E. Fernqvist-Forbes, et al. (1996). C-peptide improves autonomic nerve function IDDM patients. Diabetologia 39: 687-695.
Johansson, B.-L., B. Linde and J. Wahren (1992a). Effects of C-peptide on blood flow, capillary diffusion capacity and glucose utilization in the exercising forearm of Type I (insulin-dependent) diabetic patients. Diabetologia 35: 1151-1158.
Johansson, B.-L., S. Sjöberg and J. Wahren (1992b). The influence of human C-peptide on renal function and glucose utilization in Type I (insulin-dependent) diabetic patients. Diabetologia 35: 121-128.
Johansson, B.-L., J. Sundell, K. Ekberg, et al. (2004). C-peptide improves adenosine-induced myocardial vasodilation in type 1 diabetes patients. Am J Physiol Endocrinol Metab 286(1): E14-E19.
Kitamura, T., K. Kimura, K. Makondo, et al. (2003). Proinsulin C-peptide increases nitric oxide production by enhancing mitogen-activated protein-kinase-dependent transcription of endothelial nitric oxide synthase in aortic endothelial cells of Wistar rats. Diabetologia 46(12): 1698-1705.
Ohtomo, Y., A. Aperia, B. Sahlgren, B.-L. Johansson and J. Wahren (1996). C-peptide stimulates rat renal tubular Na⁺,K⁺-ATPase activity in synergism with neuropeptide Y. Diabetologia 39: 199-205.
Polonsky, K., B. Given and E. van Cauter (1988). Twenty-four-hour profiles and pulsatile patterns of insulin secretion in normal and obese subjects. J. Clin. Invest. 81: 442-448.
Rigler, R., A. Pramanik, P. Jonasson, et al. (1999). Specific binding of proinsulin C-peptide to human cell membranes. Proc Natl Acad Sci USA 96: 13318-13323.
Shafqat, J., L. Juntti-Berggren, Z. Zhong, et al. (2002). Proinsulin C-peptide and its analogues induce intracellular Ca²⁺ increases in human renal tubular cells. Cell Mol Life Sci 59: 1185-1189.
Sima, A. A. and K. Sugimoto (1999). Experimental diabetic neuropathy: an update. Diabetologia 42: 773-788.
Sima, A. A., W. Zhang, K. Sugimoto, et al. (2001). C-peptide prevents and improves chronic type 1 diabetic polyneuropathy in the BB/Wor rat. Diabetologia 44: 889-897.
Sundkvist, G., L. Almer and B. Lilja (1979). Respiratory influence on heart rate in diabetes mellitus. Br Med J 1(6168): 924-925.
Wahren, J., K. Ekberg, J. Johansson, et al. (2000). Role of C-peptide in human physiology. Am J Physiol Endocrinol Metab 278: E759-E768.
Wallerath, T., T. Kunt, T. Forst, et al. (2003). Stimulation of endothelial nitric oxide synthase by proinsulin C-peptide. Nitric Oxide 9: 95-102.
Zhong, Z., A. Davidescu, I. Ehrén, et al. (2005). C-peptide stimulates ERK1/2 and JNK MAP-kinases via activation of PKC in human renal tubular cells. Diabetelogia 48: 187-197.
Zhong, Z., O. Kotova, A. Davidescu, et al. (2004). C-peptide stimulates Na⁺,K⁺-ATPase via activation of ERK1/2 MAP kinases in human renal tubular cells. Cell Mol Life Sci 61: 2782-2790.

We claim:

1. A method of treating erectile dysfunction in a patient, wherein said patient has insulin-dependent diabetes, comprising the step of administering to said patient in need of such treatment a therapeutic dose of C-peptide.

2. The method of claim 1, wherein said patient has at least one long term complication of type 1 diabetes.

3. The method of claim 2, wherein said patient has peripheral neuropathy.

4. The method of claim 2, wherein said patient has autonomic neuropathy.

5. The method of claim 2, wherein said therapeutic dose of C-peptide comprises a daily dose ranging from about 1.5 to about 4.5 mg per 24 hours.

6. The method of claim 2, wherein said therapeutic dose of C-peptide comprises a daily dose ranging from about 0.3 mg to about 1.5 mg per 24 hours.

7. The method of claim 2, wherein said therapeutic dose of C-peptide comprises a daily dose ranging from about 3.0 mg to about 6 mg per 24 hours.

8. The method of any of claims 1 to 7, wherein said therapeutic dose of C-peptide maintains an average steady state concentration of C-peptide in said patient's plasma of between about 0.2 nM and about 6 nM.

9. The method of any of claims 1 to 7, wherein said therapeutic dose of C-peptide, is administered orally, intravenously, topically, sublingually, or buccally.

10. The method of any of claims 1 to 7, wherein said therapeutic dose of C-peptide is administered subcutaneously.

11. The method of any of claims 1 to 7, wherein said therapeutic dose of C-peptide is administered as a sustained release composition.

12. The method of any of claims 1 to 7, wherein said C-peptide is PEGylated.

13. A method of treating erectile dysfunction in a patient, wherein said patient has insulin-dependent diabetes, comprising administering to the patient a therapeutic dose of C-peptide, in combination with a second active agent.

14. The method of claim 13, wherein said second active agent is selected from the group consisting of a type V phosphodiesterase inhibitor, apomorphine, testosterone undecanoate, and L-arginine.

15. The method of claim 14, wherein said second active agent is a type V phosphodiesterase (PDE-5) inhibitor.

16. The method of claim 15, wherein said type V phosphodiesterase inhibitor is selected from the group consisting of sildenafil, tadalafll, vardenafil, zaprinast and pharmaceutically acceptable salts thereof.

17. The method of claim 16, wherein said type V phosphodiesterase inhibitor is sildenafil, or a pharmaceutically acceptable salt thereof.

18. The method of claim 17, wherein said type V phosphodiesterase inhibitor is sildenafil citrate.

19. The method of claim 16, wherein said type V phosphodiesterase inhibitor is tadalafll, or a pharmaceutically acceptable salt thereof.

20. The method of claim 16, wherein said type V phosphodiesterase inhibitor is vardenafil, or a pharmaceutically acceptable salt thereof.

21. The method of claim 16, wherein said type V phosphodiesterase 5 inhibitor is zaprinast, or a pharmaceutically acceptable salt thereof.

22. The method of claim 14, wherein said therapeutic dose of C-peptide, is administered subcutaneously and the type 5 phosphodiesterase (PDE-5) inhibitor is administered orally, intravenously, sublingually, or buccally.

23. The method of claim 13, wherein said patient has at least one long term complication of type 1 diabetes.

24. The method of claim 13, wherein said patient has peripheral neuropathy.

25. The method of claim 13, wherein said patient has autonomic neuropathy.

26. The method of any of claims 13 to 25, wherein said therapeutic dose of C-peptide comprises a daily dose ranging from about 1.5 to about 4.5 mg per 24 hours.

27. The method of any of claims 13 to 25, wherein said therapeutic dose of C-peptide maintains an average steady state concentration of C-peptide in said patient's plasma of between about 0.2 nM and about 6 nM.

28. The method of any of claims 13 to 25, wherein said therapeutic dose of C-peptide is administered as a sustained release composition.

29. The method of any of claims 13 to 25, wherein said C-peptide is PEGylated.

30. A method of enhancing PDE-5 inhibitor-induced relaxation of human corpus cavernosum tissue in a patient receiving a PDE-5 inhibitor, wherein said patient has diabetes, comprising administering to said patient a therapeutic dose of C-peptide, wherein PDE-5 inhibitor-induced relaxation of human corpus cavernosum tissue is enhanced compared to treatment with a PDE-5 inhibitor alone.

31. The method of claim 30, wherein said PDE-5 inhibitor is sildenafil, or a pharmaceutically acceptable salt thereof.

32. The method of claim 30, wherein said patient has insulin-dependent diabetes.

33. The method of claim 30, wherein said patient has at least one long term complication of diabetes.

34. The method of claim 30, wherein said patient has autonomic neuropathy.

35. The method of claim 30, wherein said therapeutic dose of C-peptide maintains an average steady state concentration of C-peptide in said patient's plasma above about 0.2 nM.

36. The method of claim 35, wherein said therapeutic dose of C-peptide is administered as a sustained release composition.

37. The method of claim 35, wherein said C-peptide is PEGylated.

38. A method of enhancing PDE-5 inhibitor-mediated dilation of human penile resistance blood vessels in a patient receiving a PDE-5 inhibitor, wherein said patient has diabetes, comprising administering to said patient a therapeutic dose of C-peptide, wherein dilation of said human penile resistance blood vessels is enhanced as compared to the dilation level that occurs with PDE-5 inhibitor administration alone.

39. The method of claim 38, wherein said PDE-5 inhibitor is sildenafil, or a pharmaceutically acceptable salt thereof.

40. The method of claim 38, wherein said patient has insulin-dependent diabetes.

41. The method of claim 38, wherein said patient has at least one long term complication of diabetes.

42. The method of claim 38, wherein said patient has autonomic neuropathy.

43. The method of claim 38, wherein said therapeutic dose of C-peptide maintains an average steady state concentration of C-peptide in said patient's plasma above about 0.2 nM.

44. The method of claim 43, wherein said therapeutic dose of C-peptide is administered as a sustained release composition.

45. The method of claim 43, wherein said C-peptide is PEGylated.
